(19) United States
(12) Patent Application Publication
(10) Pub. No.: US 2025/0035639 A1
(43) Pub. Date: Jan. 30, 2025

(54) PROTEIN QUANTIFICATION, TRACKING, AND IDENTIFICATION VIA PEPTIDE BARCODES

(71) Applicant: Quantum-Si Incorporated, Branford, CT (US)

(72) Inventors: Marco Ribezzi-Crivellari, Paris (FR); Sebastian Hutchinson, Branford, CT (US); Brian Reed, Madison, CT (US); Christian Francis LaPointe, Branford, CT (US)

(73) Assignee: Quantum-Si Incorporated, Branford, CT (US)

(21) Appl. No.: 18/783,280

(22) Filed: Jul. 24, 2024

Related U.S. Application Data
(60) Provisional application No. 63/659,249, filed on Jun. 12, 2024.
(60) Provisional application No. 63/569,588, filed on Mar. 25, 2024.
(60) Provisional application No. 63/515,580, filed on Jul. 25, 2023.

(51) Int. Cl. G01N 33/68; G01N 33/58

(52) U.S. Cl. CPC G01N 33/6818; G01N 33/582

(57) ABSTRACT

The present disclosure provides methods and compositions for protein quantification using peptide barcodes, sample preparation for protein quantification, protein quantification using error-resistant peptide barcodes, methods to manipulate barcoded proteins, methods to determine the binding affinities of many protein variants in a mixture of proteins by single molecule measurements.

### CROSS-REFERENCE TO RELATED APPLICATIONS

This application claims priority under 35 U.S.C. § 119(e) to U.S. Provisional Patent Application No. 63/515,580, filed Jul. 25, 2023, U.S. Provisional Patent Application No. 63/569,588, filed Mar. 25, 2024, and U.S. Provisional Patent Application No. 63/659,249, filed Jun. 12, 2024, each of which is hereby incorporated by reference in its entirety.

### REFERENCE TO AN ELECTRONIC SEQUENCE LISTING

The contents of the electronic sequence listing (R070870169US03-SEQ-KVC.xml; Size: 65,269 bytes; and Date of Creation: Jul. 23, 2024) is herein incorporated by reference in its entirety.

### BACKGROUND

Proteins are the main structural and functional components of cells, driving key biological and cellular processes. Next-generation DNA sequencing technologies have revolutionized our understanding of heredity and gene regulation, but the complex and dynamic states of cells are not fully captured by the genome and transcriptome. Applying similar approaches to proteomics has been difficult because of the scale, dynamic range, and inability to amplify the source.

### SUMMARY

Aspects of the present disclosure relate to a method of identifying a protein of interest, the method comprising: providing a fusion polypeptide comprising a protein of interest fused to a peptide barcode, wherein the peptide barcode is indicative of the protein of interest; contacting the fusion polypeptide with a cleaving agent, wherein the cleaving agent cleaves the peptide barcode from the fusion polypeptide comprising the protein of interest; and sequencing the peptide barcode to identify the protein of interest.

Aspects of the present disclosure relate to a method of identifying a protein of interest, the method comprising: expressing a fusion polypeptide comprising a protein of interest fused to a peptide barcode, wherein the peptide barcode is indicative of the protein of interest; contacting the fusion polypeptide with a cleaving agent, wherein the cleaving agent cleaves the peptide barcode from the fusion polypeptide comprising the protein of interest; and sequencing the peptide barcode to identify the protein of interest.

In some embodiments, expressing the fusion polypeptide comprises expressing an expression cassette comprising a gene and a nucleic acid barcode, wherein the gene encodes the protein of interest and the nucleic acid barcode encodes the peptide barcode, wherein expressing the expression cassette produces the fusion polypeptide.

In some embodiments, the sequencing comprises detecting a series of signals specific to the peptide barcode. In some embodiments, the sequencing comprises sequencing the peptide barcode at a single molecule level.

Aspects of the present disclosure relate to a method of protein quantification, the method comprising: expressing an expression cassette comprising a gene and a nucleic acid barcode, wherein the gene encodes a protein of interest and the nucleic acid barcode encodes a peptide barcode, wherein expressing the expression cassette produces the protein of interest conjugated to the peptide barcode; contacting the protein of interest conjugated to the peptide barcode with a cleaving agent, wherein the cleaving agent removes the peptide barcode from the protein of interest; and sequencing the peptide barcode by detecting a series of signals specific to the peptide barcode.

In some embodiments, the method further comprises contacting the peptide barcode, prior to sequencing, with a conjugating agent, wherein the conjugating agent conjugates the peptide barcode to a linker. In some embodiments, the linker immobilizes the peptide barcode in a reservoir.

In some embodiments, the series of signals specific to the peptide barcode are generated by contacting the peptide barcode with one or more barcode recognition molecules. In some embodiments, at least one signal within the series of signals is separated from another signal within the series of signals by an interpulse duration (IPD), pulse duration, and/or fluorescence lifetime that is characteristic of an association rate of barcode recognition molecule binding.

In some embodiments, at least one of the one or more barcode recognition molecules comprises a detectable label. In some embodiments, the detectable label is a luminescent label, a fluorescent label, or a conductivity label. In some embodiments, the detectable label is a fluorophore or a dye.

In some embodiments, the method further comprises, after contacting the peptide barcode with the one or more barcode recognition molecules: contacting the peptide barcode with a cleaving protein, wherein the cleaving protein removes one amino acid residue from the peptide barcode, allowing another barcode recognition molecule to contact the peptide barcode. In some embodiments, the cleaving protein is an aminopeptidase.

In some embodiments, expressing is in vivo or in vitro.

Aspects of the present disclosure relate to a method of sample preparation, the method comprising: expressing a fusion polypeptide comprising a protein of interest fused to a first tag and a peptide barcode, wherein the first tag and the peptide barcode are fused to different termini of the protein of interest; contacting the fusion polypeptide with a second tag under conditions suitable for conjugating the second tag to the peptide barcode, forming a first complex; immobilizing the first complex to an immobilizing support that binds the first tag; contacting the immobilized first complex with a third tag under conditions suitable for conjugating the third tag to the second tag, forming an immobilized second complex; and contacting the immobilized second complex with a cleaving enzyme that removes the peptide barcode conjugated to the second and third tags from the immobilized second complex comprising the protein of interest.

Aspects of the present disclosure relate to a method of sample preparation, the method comprising: expressing an expression cassette comprising a first tag, a gene, and a nucleic acid barcode, wherein the gene encodes a protein of interest and the nucleic acid barcode encodes a peptide barcode, wherein expressing the expression cassette produces the protein of interest conjugated to the first tag and the peptide barcode; contacting the protein of interest conjugated to the first tag and the peptide barcode with a second tag and a tagging enzyme and incubating the protein of interest conjugated to the first tag and the peptide barcode with the second tag and the tagging enzyme for a sufficient time to allow the tagging enzyme to enzymatically conjugate the second tag to the peptide barcode, forming a first complex; contacting the first complex with an immobilizing support that binds the first tag and incubating the first complex with the immobilizing support for a sufficient time to allow the immobilizing support to bind the first tag; contacting the immobilized first complex with a third tag and incubating the immobilized first complex with the third tag for a sufficient time to allow the third tag and the first complex to undergo a chemical reaction that binds the third tag to the first complex, forming an immobilized second complex; contacting the immobilized second complex with a cleaving enzyme that removes the peptide barcode bound to the second and third tags from the immobilized protein of interest; sequencing the peptide barcode by detecting a series of signals specific to the peptide barcode.

In some embodiments, the first tag comprises a protein tag or a ligand tag. In some embodiments, the first tag comprises a protein tag, and the immobilizing support comprises a binding partner of the protein tag. In some embodiments, the binding partner of the protein tag is a ligand or a protein that binds the protein tag. In some embodiments, the first tag comprises a Halo tag.

In some embodiments, the second tag comprises a substrate for the tagging enzyme. In some embodiments, the tagging enzyme is Sortase A. In some embodiments, the second tag comprises a substrate for Sortase A. In some embodiments, the second tag comprises a Sortase A-reactive nucleophile. In some embodiments, the second tag comprises a triglycine peptide. In some embodiments, the second tag comprises a triglycine-azide.

In some embodiments, the immobilizing support is a microbead. In some embodiments, the microbead is a nickel bead, a nickel-charged affinity resin, an agarose bead, or an iron-based bead.

In some embodiments, the third tag comprises a protein tag. In some embodiments, the third tag comprises a streptavidin protein. In some embodiments, the third tag comprises a click chemistry moiety. In some embodiments, the click chemistry moiety is a dibenzocyclooctyl (DBCO) moiety, a trans-cyclooctene (TCO) moiety, a tetrazine moiety, an azide moiety, an alkyne moiety, an aldehyde moiety, an isocyanate moiety, an N-hydroxysuccinimide moiety, a thiol moiety, an alkene moiety, a bicyclononyne moiety, or a thiamine pyrophosphate moiety. In some embodiments, the chemical reaction is click chemistry.

In some embodiments, the cleaving enzyme is a protease (e.g., an endopeptidase).

In some embodiments, the third tag immobilizes the peptide barcode in a reservoir.

In some embodiments, the series of signals specific to the peptide barcode are generated by contacting the peptide barcode with one or more barcode recognition molecules. In some embodiments, at least one signal within the series of signals is separated from another signal within the series of signals by an interpulse duration (IPD), pulse duration, and/or fluorescence lifetime that is characteristic of an association rate of barcode recognition molecule binding. In some embodiments, at least one of the one or more barcode recognition molecules comprises a detectable label. In some embodiments, the detectable label is a luminescent label, a fluorescent label, or a conductivity label. In some embodiments, the detectable label is a fluorophore or a dye.

In some embodiments, the method further comprises, after the peptide barcode has been contacted with the one or more barcode recognition molecules: contacting the peptide barcode with a cleaving protein, wherein the cleaving protein removes one amino acid residue from the peptide barcode, allowing another barcode recognition molecule to contact the peptide barcode. In some embodiments, the cleaving protein is an aminopeptidase.

In some embodiments, expressing is in vivo or in vitro.

Aspects of the present disclosure relate to a method of sequencing error-resistant peptide barcodes, the method comprising: (i) contacting a peptide barcode with one or more recognition molecules, wherein each recognition molecule recognizes one or more amino acid residues on the peptide barcode, and wherein the peptide barcode has an exposed N-terminus amino acid residue; (ii) incubating the one or more recognition molecules with the peptide barcode for a sufficient time to allow the recognition molecule that recognizes the N-terminus amino acid to bind the N-terminus amino acid and produce a signal specific to the recognition molecule; (iii) contacting the peptide barcode with a cleaving agent that removes the N-terminus amino acid and exposes a subsequent N-terminus amino acid.

In some embodiments, the method further comprises repeating steps (ii) and (iii) until each amino acid residue on the peptide barcode has been exposed and contacted with its corresponding recognition molecule; wherein the peptide barcode is incubated with five separate recognition molecules; wherein: the first recognition molecule recognizes leucine (L), isoleucine (I), and valine (V); the second recognition molecule recognizes arginine (R); the third recognition molecule recognizes phenylalanine (F), tyrosine (Y), and tryptophan (W); the fourth recognition molecule recognizes glutamine (Q) and asparagine (N); and the fifth recognition molecule recognizes alanine (A) and serine (S).

In some embodiments, the method further comprises applying a statistical model to correlate the peptide barcode to a series of signals produced by the interaction between one recognition molecule and one exposed N-terminus amino acid residue.

In some embodiments, the peptide barcode will produce the same series of signals if a subset of amino acids are switched to another subset of amino acids. In some embodiments, the peptide barcode will produce the same series of signals if: the peptide barcode comprises a leucine (L) and the leucine (L) is changed to an isoleucine (I) or a valine (V); the peptide barcode comprises an isoleucine (I) and the isoleucine is changes to a leucine (L) or a valine (V); the peptide barcode comprises a valine (V) and the valine (V) is changed to a leucine (L) or an isoleucine (I); the peptide barcode comprises a phenylalanine (F) and the phenylalanine (F) is changed to a tyrosine (Y) or a tryptophan (W); the peptide barcode comprises a tyrosine (Y) and the tyrosine (Y) is changed to a phenylalanine (F) or a tryptophan (W); the peptide barcode comprises a tryptophan (W) and the tryptophan (W) is changed to a phenylalanine (F) or a tyrosine (Y); the peptide barcode comprises a glutamine (Q) and the glutamine (Q) is changed to an asparagine (N); the peptide barcode comprises an asparagine (N) and the asparagine (N) is changed to a glutamine (Q); the peptide barcode comprises an alanine (A) and the alanine (A) is changed to a serine (S); and/or the peptide barcode comprises a serine (S) and the serine (S) is changed to an alanine (A).

Aspects of the present disclosure relate to a method comprising: expressing a library of fusion polypeptides, each fusion polypeptide of the library comprising a protein of interest fused to a peptide barcode, wherein the peptide barcode is indicative of the protein of interest to which it is fused; attaching to the fusion polypeptides of the library of fusion polypeptides a loading complex; immobilizing the library of fusion polypeptides to a surface; screening the library of fusion polypeptides for biological activity; selecting one or more fusion polypeptides of the library of fusion polypeptides based on biological activity; contacting the library of fusion polypeptides with a labeled binding reagent; detecting signals indicative of binding between the labeled binding reagent and the protein of interest of at least one fusion polypeptide of the library; cleaving the peptide barcode from the at least one fusion polypeptide comprising the protein of interest; sequencing the peptide barcode to identify the protein of interest of the at least one fusion polypeptide.

In some embodiments, the loading complex is a dibenzocyclooctyl (DBCO) moiety.

In some embodiments, the biological activity is antigen binding affinity, antibody binding affinity, receptor binding affinity, target protein binding affinity, or another biological activity.

Aspects of the present disclosure relate to a method comprising: expressing a library of fusion polypeptides, each fusion polypeptide of the library comprising a protein of interest fused to a peptide barcode, wherein the peptide barcode is indicative of the protein of interest to which it is fused; immobilizing the library of fusion polypeptides to a surface; contacting the library of fusion polypeptides with a labeled binding reagent; detecting signals indicative of binding between the labeled binding reagent and the protein of interest of at least one fusion polypeptide of the library; cleaving the peptide barcode from the at least one fusion polypeptide comprising the protein of interest; sequencing the peptide barcode to identify the protein of interest of the at least one fusion polypeptide; and determining one or more biophysical characteristics of the protein of interest of the at least one fusion polypeptide based on the detecting and the sequencing.

Aspects of the present disclosure relate to a method of identifying a target analyte in a region of a sample, the method comprising: (i) contacting a sample with an affinity reagent conjugated to a peptide barcode indicative of a target analyte to which the affinity reagent binds; (ii) releasing the peptide barcode from the affinity reagent in a first region of the sample; and (iii) sequencing the peptide barcode to identify the target analyte in the first region of the sample.

In some embodiments, after step (ii), the peptide barcode remains conjugated to the affinity reagent in a second region of the sample. In some embodiments, the method further comprises releasing the peptide barcode from the affinity reagent in the second region of the sample. In some embodiments, the sequencing comprises identifying the target analyte in each of the first and second regions of the sample.

In some embodiments, the target analyte is a protein or a nucleic acid. In some embodiments, the target analyte is a monomeric or multimeric protein. In some embodiments, the target analyte is a DNA or RNA molecule (e.g., mRNA). In some embodiments, the target analyte is a gene transcript.

In some embodiments, the affinity reagent is an antibody or antigen-binding fragment thereof, a nanobody, an aptamer, or an antisense oligonucleotide. In some embodiments, the affinity reagent is an antibody-drug conjugate (ADC). In some embodiments, the affinity reagent is an immunohistochemistry (IHC)-compatible antibody.

In some embodiments, the peptide barcode is conjugated to the affinity reagent via a linker.

In some embodiments, the linker comprises a cleavage site (e.g., a protease cleavage site). In some embodiments, the peptide barcode is released from the affinity reagent by contacting the linker with a cleaving agent (e.g., a cleaving agent that cleaves the linker at the cleavage site). In some embodiments, the cleaving agent is a cleaving enzyme (e.g., a protease). In some embodiments, the cleaving enzyme is an endopeptidase.

In some embodiments, the linker is a photocleavable linker. In some embodiments, the peptide barcode is released from the affinity reagent by exposing the linker to photocleaving light (e.g., a light source that cleaves the photocleavable linker). In some embodiments, the photocleaving light is ultraviolet radiation or a laser.

In some embodiments, the method further comprises washing the sample between step (i) and step (ii).

In some embodiments, the method further comprises imaging the sample prior to sequencing the peptide barcode. In some embodiments, the sequencing comprises determining a concentration of the target analyte in the first region of the sample. In some embodiments, the sequencing generates sequencing reads associated with the presence of the target analyte in the first region of the sample. In some embodiments, the method further comprises mapping the sequencing reads to a spatial representation (e.g., an image) of the sample.

In some embodiments, the sample is a biological sample. In some embodiments, the biological sample is fixed. In some embodiments, the biological sample is a cell sample. In some embodiments, the biological sample is a serum sample. In some embodiments, the biological sample is a tissue sample. In some embodiments, the tissue sample is a formalin-fixed paraffin-embedded (FFPE) tissue sample.

In some embodiments, the sample is contacted with a plurality of affinity reagents conjugated to different peptide barcodes indicative of different target analytes.

Aspects of the present disclosure relate to a method of identifying target analytes in a sample, the method comprising: (i) contacting a sample with a plurality of affinity reagents conjugated to different peptide barcodes indicative of different target analytes; (ii) releasing one or more peptide barcodes from one or more respective affinity reagents in a first region of the sample; (iii) releasing one or more peptide barcodes from one or more respective affinity reagents in a second region of the sample; and (iv) sequencing the one or more peptide barcodes released from each of the first and second regions of the sample.

In some embodiments, the sequencing comprises determining an abundance of at least one target analyte in the first region relative to the second region. In some embodiments, the sequencing generates sequencing reads associated with the presence of different target analytes in the first and second regions of the sample. In some embodiments, the method further comprises mapping the sequencing reads to a spatial representation of the sample. In some embodiments, the spatial representation is indicative of localization of different target analytes in different regions of the sample.

Aspects of the present disclosure relate to a method of identifying a therapeutic agent in a region of a sample, the method comprising: (i) contacting a sample with a therapeutic agent conjugated to a peptide barcode indicative of the therapeutic agent; (ii) releasing the peptide barcode from the therapeutic agent in a first region of the sample; and (iii) sequencing the peptide barcode to identify the therapeutic agent in the first region of the sample. In some embodiments, the therapeutic agent comprises an affinity reagent as described herein (e.g., an ADC). In some embodiments, the peptide barcode is conjugated to the therapeutic agent via a linker as described herein. In some embodiments, the sample is a biological sample (e.g., a cell sample, a serum sample, a tissue sample).

In some embodiments, after step (ii), the peptide barcode remains conjugated to the therapeutic agent in a second region of the sample. In some embodiments, the method further comprises releasing the peptide barcode from the therapeutic agent in the second region of the sample. In some embodiments, the sequencing comprises identifying the therapeutic agent in each of the first and second regions of the sample. In some embodiments, the method further comprises washing the sample between step (i) and step (ii).

Aspects of the present disclosure relate to a method of evaluating an antibody-drug conjugate (ADC) in a subject, the method comprising: (i) providing a sample of a subject receiving an ADC, wherein the ADC is conjugated to a peptide barcode; (ii) releasing the peptide barcode from the ADC in the sample; (iii) sequencing the peptide barcode; and (iv) evaluating the ADC based on the sequencing. In some embodiments, the sample is a serum sample of the subject. In some embodiments, the sample is a tissue sample of the subject. In some embodiments, the subject is a human. In some embodiments, the subject is a non-human animal.

In some embodiments, the evaluating comprises determining a concentration of the ADC in the sample based on the sequencing. In some embodiments, the evaluating comprises comparing the concentration of the ADC in the sample to a control sample. In some embodiments, the control sample is a sample of the subject prior to receiving the ADC. In some embodiments, the control sample is a sample of the subject at a different time point after receiving the ADC.

In some embodiments, step (ii) comprises: releasing the peptide barcode from the ADC in a first region of the sample, wherein the peptide barcode remains conjugated to the ADC in a second region of the sample. In some embodiments, the method further comprises releasing the peptide barcode from the ADC in the second region of the sample. In some embodiments, the sequencing comprises sequencing the peptide barcode released from the ADC in each of the first and second regions of the sample. In some embodiments, the evaluating comprises: determining a first concentration of the ADC in the first region of the sample based on the sequencing; and determining a second concentration of the ADC in the second region of the sample based on the sequencing.

Aspects of the present disclosure relate to a method of identifying delivery to a target cell, the method comprising: contacting a cell with a delivery agent that comprises a polynucleotide, wherein the polynucleotide encodes a protein of interest fused to a peptide barcode; contacting the cell with a cleaving agent configured to cleave the peptide barcode from the protein of interest; and detecting the peptide barcode to identify uptake of the polynucleotide by the cell. In some embodiments, the delivery agent is a lipid nanoparticle that encapsulates the polynucleotide. In some embodiments, the contacting is performed under conditions suitable for expression of the polynucleotide within the cell.

Aspects of the present disclosure relate to a method of evaluating nanoparticle delivery to a target cell, the method comprising: contacting a cell with a plurality of nanoparticles having different lipid compositions, wherein each nanoparticle encapsulates a polynucleotide, wherein the polynucleotide encodes a protein of interest fused to a peptide barcode indicative of lipid composition of the nanoparticle; contacting the cell with a cleaving agent configured to cleave the peptide barcode from the protein of interest; and sequencing the peptide barcode to identify the lipid composition of the nanoparticle. In some embodiments, the contacting is performed under conditions suitable for expression of the polynucleotide within the cell.

Aspects of the present disclosure relate to a method of evaluating protein production, the method comprising: contacting a cell with an RNA (e.g., mRNA) molecule encoding a protein of interest and a peptide barcode; contacting the cell with a cleaving agent configured to cleave the peptide barcode from the protein of interest; and detecting the peptide barcode to identify production (e.g., expression) of the protein of interest. In some embodiments, the contacting is performed under conditions suitable for expression of the polynucleotide within the cell.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIG. 1 shows A) Generation of Barcoded protein libraries 1) Protein Genes are assembled as a fusion of the Protein Of Interest (POI) with a specific 3′ sequence coding for a peptide barcode. 2) Barcoded protein libraries are expressed in-vitro or in-vivo and can be subject to experiments, e.g. screening or selection. 3) Barcodes are cleaved and conjugated to a macromolecular linker. 4) Barcode libraries are sequenced on a sequencing instrument. B) Representative traces of 3 peptide barcodes exemplifying error correction. The traces are distinctive and error tolerant.

FIG. 2 shows A) The model library. NB1, an MBP binder is associated with barcode 1 (BC1), NB2, a GFP binder is associated with Barcode 2 (BC2). B) Upper panel: the selection process. After saving a fraction of the library for direct sequencing, the library is incubated with beads displaying GFP. Beads are then pelleted and washed. The barcodes are then eluted by proteolysis and sequenced on a sequencing device. The summary of barcode Quantitation is shown in the lower panel.

FIGS. 3A-3D show the mechanistic steps of library preparation using Sortase A.

FIG. 4 shows an example Sortase A library preparation.

FIG. 5 shows an example protein construct used during Sortase A library preparation.

FIG. 6 shows a workflow for Sortase A library preparation.

FIG. 7 shows a workflow for Sortase A library preparation.

FIG. 8 shows a protein construct developed for use in Sortase A library preparation.

FIG. 9 shows a developed workflow for Sortase A library preparation. The triangle shape in the top left panel represents a triglycine (GGG) appended to either a linker-streptavidin group (-Q24-SV) or an azide group (—N₃).

FIG. 10 shows an example protein construct used during Sortase A library preparation.

FIG. 11 shows a workflow for Sortase A library preparation.

FIG. 12 shows expression of peptide barcodes in T7 Shuffle cells.

FIG. 13 shows an overview of sample preparation using the enzymatic method described herein.

FIG. 14 shows the Sortase A reaction.

FIG. 15 shows possible Sortase A nucleophiles.

FIG. 16 shows an overview of sample preparation using a 1-step enzymatic method or a 2-step enzymatic method.

FIGS. 17A-17B show workflows that may be employed to analyze proteins of interest.

FIG. 18 shows an example peptide barcode. Each circle represents an individual amino acid.

FIG. 19 shows example signals produced by sequencing peptide barcodes.

FIG. 20 shows an example overview of real-time dynamic protein sequencing. Protein samples are digested into peptide fragments, immobilized in nanoscale reaction chambers, and incubated with a mixture of freely-diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs).

FIG. 21 shows properties of example regions of interest (ROIs) of peptide barcodes.

FIG. 22 shows a workflow for peptide barcode design.

FIG. 23 shows discretization of peptide barcodes based on fluorescence lifetime.

FIG. 24 shows a workflow for converting a peptide barcode from a predicted kinetic signature into a barcode string.

FIG. 25 shows discretization of peptide barcodes based on fluorescence lifetime and pulse width.

FIG. 26 shows a workflow for converting a color only discretization of peptide barcodes to a barcode string.

FIG. 27 shows an example peptide barcode generation algorithm.

FIG. 28 shows an example peptide barcode design iteration.

FIG. 29 shows an example peptide barcode design iteration.

FIG. 30 shows an example peptide barcode design iteration.

FIG. 31 shows an example peptide barcode design iteration.

FIG. 32 shows an example peptide barcode design iteration.

FIG. 33 shows an example peptide barcode design iteration.

FIG. 34 shows a quantitative assessment of peptide barcode design iterations.

FIG. 35 shows an example application of peptide barcodes.

FIG. 36 shows an example application of peptide barcodes.

FIG. 37 shows traces for the five highest performing peptide barcodes.

FIG. 38 shows additional quantitative analyses for peptide barcodes.

FIG. 39 shows additional quantitative analyses for peptide barcodes.

FIG. 40 shows ten high-performing peptide barcodes.

FIG. 41 shows example peptide barcode traces.

FIG. 42 shows an example of nanobody section for peptide barcodes.

FIG. 43 shows parallelized antibody binding kinetics using peptide barcodes.

FIG. 44 shows parallelized antibody binding kinetics using peptide barcodes.

FIG. 45 shows example plasmids, as described herein.

FIG. 46 shows an example plasmid, as described herein.

FIG. 47 shows an example protein construct used during Sortase A library preparation.

FIG. 48 shows nine unique peptide barcodes ranging from six to ten amino acids in length.

FIG. 49 shows the relative abundance of the nine unique peptide barcodes shown in FIG. 48 in a pooled screen.

FIG. 50 shows the scalability of the peptide barcodes described herein.

FIG. 51 shows a workflow for a nanobody enrichment application of the peptide barcodes described herein.

FIG. 52 shows a method of screening drug delivery methods using the peptide barcodes described herein.

### DETAILED DESCRIPTION

Aspects of the disclosure relate to compositions and methods for peptide sequencing, protein quantification via peptide barcodes, enzymatic library preparation (also referred to as sample preparation), and peptide barcode design and application.

Aspects of the present disclosure relate to protein sequencing via peptide barcodes. A peptide barcode is an amino acid sequence expressed alone or appended to a molecule of interest, such as a protein of interest or an affinity reagent. In some embodiments, a nucleic acid sequence encoding the protein of interest or affinity reagent and peptide barcode is expressed in vitro or in vivo and a peptide barcode appended to a protein of interest or affinity reagent is produced. In some embodiments, the nucleic acid is expressed as an expression cassette. In some embodiments, the method further comprises expressing a plurality of expression cassettes comprising a gene (e.g., a nucleic acid encoding a protein of interest or an affinity reagent) and a nucleic acid barcode. In some embodiments, the plurality of expression cassettes each comprise the same gene and nucleic acid barcode. In some embodiments, the plurality of expression cassettes comprise the same gene but different nucleic acid barcodes. In some embodiments, the plurality of expression cassettes comprise different genes but the same nucleic acid barcode. In some embodiments, the plurality of expression cassettes each comprise different genes and different nucleic acid barcodes.

In some embodiments, the peptide barcode is cleaved from the protein of interest or affinity reagent and only the peptide barcode is prepared for sequencing and sequenced. In some embodiments, the peptide barcode is cleaved by a cleaving agent. In some embodiments, the cleaving agent is an endopeptidase. In some embodiments, the peptide barcode further comprises a tag. In some embodiments, the peptide barcode is at least 1, at least 2, at least 3, at least 4, at least 5, at least 6, at least 7, at least 8, at least 9, at least 10, at least 15, at least 20, at least 25, at least 30, at least 35, at least 40, at least 45, or at least 55 amino acid residues in length. In some embodiments, the peptide barcode produces a signature trace when sequenced, that allows a user to correlate the peptide barcode with a protein of interest. In some embodiments, the abundance of a protein is quantified by sequencing the peptide barcode.

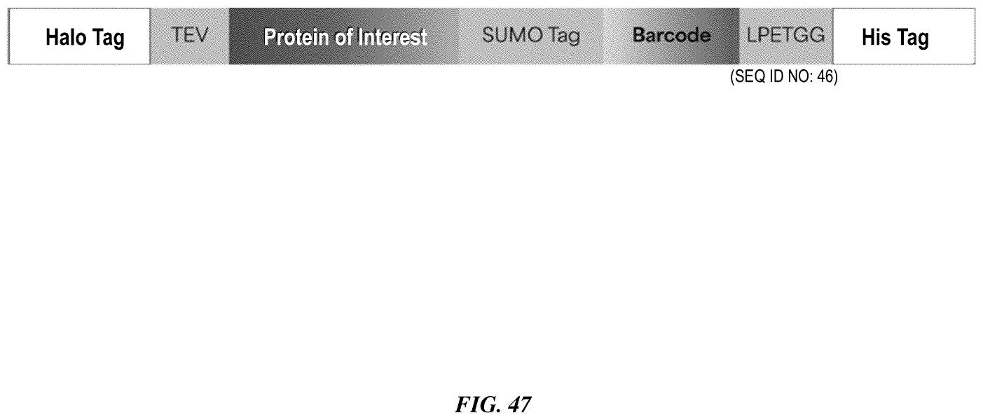

Aspects of the present disclosure relate to preparing a peptide barcode library using an enzymatic reaction. In some embodiments, a protein construct is produced comprising one or more tags and/or one or more cleavage sites for immobilizing, cleaving, and/or isolating a peptide barcode. In some embodiments, a tag is used to immobilize a protein construct onto a microbead. In some embodiments, the microbead is a “nickel-charged affinity resin.” In some embodiments, the enzyme is a Sortase. In some embodiments, the enzyme is Sortase A. In some embodiments, a His-tag is used on the protein construct to isolate or purify the peptide barcode after library preparation. In some embodiments, a tag is an affinity purification tag for use in purifying and/or immobilizing an expression product. For example, FIG. 47 shows a non-limiting protein expression construct. As shown, in some embodiments, a protein construct comprises one or more affinity purification tags, such as a HaloTag and/or a His-tag. In some embodiments, a protein construct comprises one or more tags selected from a HaloTag, His-tag, FLAG-tag, Maltose Binding Protein (MBP) tag, glutathione-S-transferase (GST) tag, MYC tag, hemagglutinin-A (HA) tag, Spytag/SpyCatcher, Strep-tag, or any combination thereof. Additional examples of tags are known in the art and described herein. Selection of the appropriate affinity purification tag will be dependent on the application and choice of host system. The process for selecting the appropriate affinity purification tag is known in the art.

In some embodiments, a peptide barcode comprises or is attached to an enzyme recognition sequence (e.g., a Sortase recognition sequence comprising the sequence “LPETGG” (SEQ ID NO: 46)), which is recognized by an enzyme described herein. In some embodiments, the enzyme adds a tag (e.g., a second tag, such as an enzyme nucleophile) to the peptide barcode. In some embodiments, the enzyme nucleophile is triglycine-PEG3-Picolyl azide, triglycine-Lys(N3), or 3-azido-1-propylamine. In some embodiments, a triglycine-azide is added to the peptide barcode at the “LPETGG” (SEQ ID NO: 46) sequence. In some embodiments, the azide is available for a further chemical reaction. In some embodiments, the further chemical reaction adds an additional tag (e.g., a third tag). In some embodiments, the further chemical reaction is a click chemistry reaction. In some embodiments, the click chemistry reaction is a copper-free click reaction. In some embodiments, after library preparation, the peptide barcode is purified or isolated for peptide sequencing.

Aspects of the present disclosure relate to a method to design amino acid sequences (“peptide barcodes”) to be used in single-molecule protein sequencing applications. In some embodiments, peptide barcodes contain one or more “information” regions and “functional” regions. In some embodiments, peptide barcodes contain only an “information” region. In some embodiments, the information region is used for peptide sequencing. In some embodiments, the functional region is used for further library preparation. In some embodiments, peptide barcodes give rise to Regions of Interest (ROIs) when sequenced. In some embodiments, an ROI represents one or more amino acid residues. ROIs stem from the on-off binding of a recognizer to the N-terminal amino acid. Transitions between ROIs depend on enzymatic cutting of the N-terminal amino-acid resulting in the exposure of a different N-terminal amino acid. Recognizers have multiple substrates, a single recognizer can bind several N-terminal amino-acids. In some embodiments, the recognizer is PS1223, PS1220, PS610, PS1259, or PS1165, which have been described (see, e.g., International Publication Nos. WO 2021/236983 and WO 2023/122769, the relevant contents of which, including recognizer sequence information, are hereby incorporated by reference in entirety).

In some embodiments, PS1223 recognizes leucine (L), isoleucine (I), and/or valine (V). In some embodiments PS1220 recognizes arginine (R). In some embodiments, PS610 recognizes phenylalanine (F), tyrosine (Y), and/or tryptophan (W). In some embodiments, PS1259 recognizes glutamine (Q) and/or asparagine (N). In some embodiments, PS1165 recognizes alanine (A) and/or serine (S).

A peptide barcode signal is interpreted via its ROIs. Each ROI is generated by one of five recognizers, labeled with a specific dye, that emits light with specific properties. In some embodiments, the dye is luminescent. In some embodiments, the dye is a fluorophore. Within an ROI, consistent pulsing by one recognizer may be observed. In some embodiments, each pulse in the ROI has a Pulse Width (PW) and an Inter Pulse Distance (IPD). The median pulse width and the median IPD are distinctive of the ROIs and sensitive to the nature of the N-terminal amino-acid and of the context (e.g., penultimate amino-acid and beyond). The signal from one aperture may be idealized as a ‘kinetic signature’ that summarizes the properties of the ROIs. Example properties that can be used are ‘color’ distinguishing the different recognizers, median PW, and median IPD.

In some embodiments, the pulsing properties of each residue along the amino acid barcode (ROIs) can be analyzed on chip. ROIs are described by multiple parameters including binratio (average or distribution), pulse width (average or distribution), interpulse distance (average or distribution). In some embodiments, the ROI is described by binratio, pulse width, an interpulse distance. In some embodiments, the ROI is described by binratio only. In some embodiments, the ROI is described by pulse width only. In some embodiments, the ROI is described by interpulse distance only. In some embodiments, ROI are discretized based on mean binratio and mean pulse width. In some embodiments, peptide barcodes with 5 ROIs give a total diversity of 625 possible barcodes (5*5*5*5). In some embodiments, barcode identity is mapped to its ROIs. In some embodiments, the abundance of a protein of interest can be quantified by sequencing peptide barcodes are analyzing peptide barcode ROIs.

### Single Molecule Peptide Barcode Sequencing

Aspects of the present disclosure relate to single molecule peptide barcode sequencing to detect (e.g., identify) one or more target analytes within a sample. Methods of identifying a target analyte within a sample using oligonucleotide barcode sequencing are described, for example, in U.S. Pat. No. 9,995,739, “PROTEIN DETECTION VIA NANOREPORTERS,” and U.S. Pat. No. 10,640,816, “SIMULTANEOUS QUANTIFICATION OF GENE EXPRESSION IN A USER-DEFINED REGION OF A CROSS-SECTIONED TISSUE,” the entire disclosure of each of which is hereby incorporated by reference in its entirety. The present disclosure relates, in part, to the use of peptide barcode sequencing to identify a target analyte within a sample. Oligonucleotide barcode sequencing is limited in use due to difficulties associated with attaching oligonucleotides to antibodies and because the readout is restricted to the number of available nucleobases (i.e., four). Because DNA is limited to four nucleobases, oligonucleotide barcodes must be lengthy to encode sufficient information to distinguish large panels of antibodies. Oligonucleotide barcode sequencing is further limited because the process of DNA sequencing often results in oligonucleotide amplification errors. In contrast, the inventors of the present disclosure have discovered a method in which peptide barcodes, and not oligonucleotide barcodes, are used to determine the identity of a target analyte within a sample. The use of peptide-based barcodes is an improvement over methods that require oligonucleotide-based barcodes because a peptide barcode can be expressed in cis with an affinity reagent, thus eliminating the laborious step of attaching an oligonucleotide-based barcode to an affinity reagent (e.g., an antibody). In addition, a short peptide barcode can encode sufficient information for use in large panels of antibodies due to the availability of twenty amino acids, as opposed to four nucleobases.

Accordingly, the present disclosure relates, at least in part, to a method of identifying a target analyte in a region of a sample using an affinity reagent conjugated to a peptide barcode. In some embodiments, a sample is contacted with an affinity reagent conjugated to a peptide barcode. In some embodiments, an affinity reagent is an antibody or antigen-binding fragment thereof, an antibody-drug conjugate (ADC), an immunohistochemistry (IHC)-compatible antibody, a nanobody, an aptamer, or an antisense oligonucleotide. In some embodiments, an affinity reagent is an antibody-drug conjugate (ADC). In some embodiments, an affinity reagent is an immunohistochemistry (IHC)-compatible antibody. In some embodiments, an affinity reagent is configured to be used in a tissue sample that has been prepared for IHC analysis. In some embodiments, an affinity reagent is an antibody that is used to bind to a target analyte and that is configured to be used in an IHC analysis. In some embodiments, an affinity reagent is specific to a target analyte, and a peptide barcode to which the affinity reagent is bound is specific to the target analyte. After an affinity reagent binds to a specific target analyte within a sample, a peptide barcode to which the affinity reagent is bound is released from the affinity reagent and sequenced. Accordingly, the presence of sequencing reads generated from sequencing a peptide barcode is indicative of the presence of a target analyte to which the peptide barcode is specific in a sample. Peptide barcode sequencing is carried out using any method of peptide sequencing described in the present disclosure. Methods of polypeptide analysis and peptide barcode sequencing are described, for example, in WO 2020/102741, “METHODS AND COMPOSITIONS FOR PROTEIN SEQUENCING,” WO 2022/132188, “MOLECULAR BARCODE ANALYSIS BY SINGLE-MOLECULE KINETICS,” WO 2023/122769, “COMPOSITIONS AND METHODS FOR POLYPEPTIDE ANALYSIS,” and WO 2024/086832, “POLYPEPTIDE CLEAVING REAGENTS AND USES THEREOF,” the entire content of each of which is hereby incorporated by reference in its entirety.

In some embodiments, an affinity reagent is an antibody-drug conjugate (ADC) comprising an antibody component and a drug component, where the ADC is conjugated to a peptide barcode as described herein. In some embodiments, the ADC is any ADC known in the art or otherwise of interest for evaluating in accordance with the disclosure. In some embodiments, the antibody component of an ADC is any antibody or antigen-binding fragment thereof described herein or known in the art. In some embodiments, the drug component of an ADC is any therapeutic compound described herein or known in the art, including, without limitation, a small molecule (e.g., a compound equal to or less than about 1,000 Daltons), a peptide or protein, a nucleic acid (e.g., RNA, including siRNA), a cytotoxic compound (e.g., a chemotherapeutic compound), or a warhead. Examples of ADCs and methods of preparing ADCs are described, for example, in Fu, Z., et al. Antibody drug conjugate: the “biological missile” for targeted cancer therapy. Sig Transduct Target Ther 7, 93 (2022); Dumontet, C., et al. Antibody-drug conjugates come of age in oncology. Nat Rev Drug Discov 22, 641-661 (2023); Tsuchikama, K., et al. Exploring the next generation of antibody-drug conjugates. Nat Rev Clin Oncol 21, 203-223 (2024); Khongorzul, P., et al. Antibody-Drug Conjugates: A Comprehensive Review. Mol Cancer Res. 2020 January; 18(1):3-19; Shastry, M., et al. Rise of Antibody-Drug Conjugates: The Present and Future. Am Soc Clin Oncol Educ Book. 2023 May; 43:e390094; Gogia, P., et al. Antibody-Drug Conjugates: A Review of Approved Drugs and Their Clinical Level of Evidence. Cancers (Basel). 2023 Jul. 30; 15(15):3886; and Riccardi, F., et al. A comprehensive overview on antibody-drug conjugates: from the conceptualization to cancer therapy. Front Pharmacol. 2023 Sep. 18; 14:1274088, the entire contents of each of which is hereby incorporated by reference in its entirety.

In some embodiments, the drug component of an ADC is conjugated to the antibody component of the ADC via a linker. In accordance with the affinity reagents and methods of use described herein, the ADC is conjugated to a peptide barcode. In some embodiments, the drug component and the peptide barcode are conjugated to different attachment sites (e.g., different amino acids) on the antibody component. In some embodiments, the drug component and the peptide barcode are conjugated to a single attachment site on the antibody component. For example, in some embodiments, the drug component is conjugated to the antibody component via the peptide barcode or via a linker comprising the peptide barcode (e.g., a photocleavable linker, a linker comprising a cleavage site).

An affinity reagent, in some embodiments, is conjugated to a peptide barcode either directly or indirectly via linker. In some embodiments, an affinity reagent is directly conjugated to a peptide barcode. In some embodiments, an affinity reagent is indirectly conjugated to a peptide barcode. In some embodiments, an affinity reagent is conjugated to a peptide barcode via a linker. In some embodiments, a linker is a peptide linker. In some embodiments, a linker is an oligonucleotide linker. In some embodiments, a peptide barcode is released from its respective affinity reagent by cleaving a linker. Methods of cleaving a linker are known in the art. For example, a linker can be cleaved by exposing the linker to light or by exposing a linker to a cleaving enzyme.

In some embodiments, a linker is a photocleavable linker. In some embodiments, a peptide barcode is released from an affinity reagent by exposing the linker to photocleaving light. In some embodiments, photocleaving light is ultraviolet radiation. In some embodiments, photocleaving light is a laser. In some embodiments, a photocleavable linker is 2-nitrobenzyl, ortho-benzene (ONB), thioacetal ortho-nitrobenzene (TNB), coumarin, cyanine, carbazole, quinoline, xanthene, ortho-hydroxy cinnamate, benzoin, or benzophenone. Photocleavable linkers are known in the art. For example, photocleavable linkers are described in US 2011/0151451, “USE OF CONJUGATES WITH LINKERS CLEAVED BY PHOTODISSECTION OR FRAGMENTATION FOR MASS SPECTROMETRY ANALYSIS OF TISSUE SECTIONS,” U.S. Pat. No. 10,266,874, “METHODS, KITS, AND SYSTEMS FOR MULTIPLEXED DETECTION OF TARGET MOLECULES AND USES THEREOF,” Choi, S. K. (2020). Photocleavable linkers: design and applications in nanotechnology. Photonanotechnology for Therapeutics and Imaging, 243-275. doi:10.1016/b978-0-12-817840-9.00009-6, and Agasti S S, Liong M, Peterson V M, Lee H, Weissleder R. Photocleavable DNA barcode-antibody conjugates allow sensitive and multiplexed protein analysis in single cells. J Am Chem Soc. 2012 Nov. 14; 134(45):18499-502, the entire disclosure of each of which is hereby incorporated by reference in its entirety.

In some embodiments, a linker comprises a cleavage site. In some embodiments, a peptide barcode is released from an affinity reagent by contacting a linker between the affinity reagent and the peptide barcode with a cleaving agent. In some embodiments, a cleaving agent is a cleaving enzyme. In some embodiments, a cleaving enzyme is a protease, such as an endopeptidase. An endopeptidase is a proteolytic peptidase that breaks peptide bonds of nonterminal amino acids. Non-limiting examples of endopeptidases include: trypsin (cuts after arginine or lysine, unless followed by a proline), chymotrypsin (cuts after phenylalanine, tryptophan, or tyrosine, unless followed by a proline), elastase (cuts after alanine, glycine, serine, or valine, unless followed by a proline), thermolysin (cuts before isoleucine, methionine, phenylalanine, tryptophan, tyrosine, or valine, unless preceded by a proline), pepsin (cuts before leucine, phenylalanine, tryptophan, or tyrosine, unless preceded by a proline), glutamyl endopeptidase (cuts after glutamate), neprilysin, and prolyl endopeptidase. The structure and function of endopeptidases and endopeptidase cleavage sites are known in the art. For example, endopeptidases are described in van der Velden V H, Hulsmann A R. Peptidases: structure, function and modulation of peptide-mediated effects in the human lung. Clin Exp Allergy. 1999 April; 29(4):445-56, the entire disclosure of which is hereby incorporated by reference in its entirety.

Accordingly, in some embodiments, a linker comprises a cleavage site comprising an amino acid sequence that is cleaved by a cleaving enzyme (e.g., a protease). In some embodiments, a cleavage site comprises at least one chemical functional group that is cleaved by a cleaving enzyme. For example, ester and carbamate functional groups can be hydrolyzed by cleaving enzymes, such as esterases and cytochrome P450. Thus, in some embodiments, the cleavage site comprises one or more chemical functional groups (e.g., one or more ester and/or carbamate functional groups), and the cleaving enzyme is an enzyme capable of cleaving the one or more chemical functional groups (e.g., an esterase). Additional examples of cleavable linkers and suitable cleaving agents are described in Hoppenz, P., et al. Peptide-Drug Conjugates and Their Targets in Advanced Cancer Therapies. Front Chem. 2020 Jul. 7; 8:571, the relevant content of which is hereby incorporated by reference in its entirety.

Aspects of the present disclosure relate to the use of an affinity reagent conjugated to a peptide barcode to detect (e.g., determine the presence or identity of) a target analyte within a sample. In some embodiments, a sample is a biological sample. In some embodiments, a biological sample is derived from a human, a non-human primate, a rodent, an insect, a parasite, or a plant. In some embodiments, a biological sample is fixed. In some embodiments, a biological sample is a cell sample. In some embodiments, a biological sample is a serum sample. In some embodiments, a biological sample is a tissue sample. In some embodiments, a tissue sample is formalin fixed. In some embodiments, a tissue sample is paraffin embedded. In some embodiments, a tissue sample is a formalin-fixed, paraffin-embedded (FFPE) tissue sample. In some embodiments, a tissue sample is a fresh FFPE tissue sample. In some embodiments, a tissue sample is a frozen FFPE tissue sample. In some embodiments, a tissue sample is a fresh-frozen FFPE tissue sample.

In some embodiments, a target analyte is a protein. In some embodiments, a target analyte is a monomeric protein. In some embodiments, a target analyte is a multimeric protein. In some embodiments, a target analyte is an antibody. In some embodiments, a target analyte is a receptor. In some embodiments, a target analyte is a ligand. In some embodiments, a target analyte is a cellular component. In some embodiments, a target analyte is a subcellular compartment. In some embodiments, a target analyte is a nucleic acid. In some embodiments, a target analyte is a DNA molecule. In some embodiments, a target analyte is a genomic DNA (gDNA) molecule. In some embodiments, a target analyte is a complementary DNA (cDNA) molecule. In some embodiments, a target analyte is an RNA molecule. In some embodiments, a target analyte is a messenger RNA (mRNA) molecule. In some embodiments, a target analyte is a transfer RNA (tRNA) molecule. In some embodiments, a target analyte is a ribosomal RNA (rRNA) molecule. In some embodiments, a target analyte is a gene transcript.

Biological samples, as described herein, contain hundreds or thousands of putative target analytes. In some embodiments, a single affinity reagent conjugated to a single peptide barcode is used to identify a single target analyte within a sample. In some embodiments, a plurality of affinity reagents, each conjugated to a peptide barcode, is used to identify a plurality of target analytes within a sample. In some embodiments, a user of the method described herein obtains information regarding the location of one or more target analytes within a sample by selectively releasing peptide barcodes from a user-specified region within the sample. In some embodiments, a user releases peptide barcodes from a first user-specified region within a sample while peptide barcodes in one or more other regions within the sample remain conjugated to their respective affinity reagents.

Aspects of the present disclosure relate to using one or more affinity reagents, each conjugated to a peptide barcode, to identify a target analyte within a sample. In some embodiments, a sample is contacted with one or more affinity reagents, each conjugated to a peptide barcode. In some embodiments, each affinity reagent of the one or more affinity reagents is specific to a different target analyte. In some embodiments, each affinity reagent of the one or more affinity reagents is specific to the same target analyte. In some embodiments, a first subset of affinity reagents of the one or more affinity reagents are specific to a first target analyte and a second subset of affinity reagents of the one or more affinity reagents are specific to a second target analyte. In some embodiments, an N number of subsets of the one or more affinity reagents are specific to an N number of target analytes.

In some embodiments, a sample is contacted with one or more affinity reagents conjugated to peptide barcodes and the sample is incubated with the one or more affinity reagents for a sufficient time to allow the one or more affinity reagents to bind to their respective target analytes. In some embodiments, following an incubation period, a sample is washed to remove unbound affinity reagents. In some embodiments, a sample is washed with a wash buffer. In some embodiments, following washing, only affinity reagents bound to their respective target analytes remain on the sample. In some embodiments, a user releases peptide barcodes from their respective affinity reagents, collects the peptide barcodes, and sequences the peptide barcodes using any peptide sequencing method described herein. In some embodiments, the presence of sequencing reads associated with a peptide barcode indicates the presence of a target analyte to which the peptide barcode is specific in a sample.

In some embodiments, peptide barcodes are released from a specific region within a sample. In some embodiments, peptide barcodes conjugated to bound affinity reagents within a first region are released and peptide barcodes conjugated to bound affinity reagents within a second region remain conjugated to the bound affinity reagents within the second region. In some embodiments, a user selectively releases peptide barcodes from affinity reagents bound to target analytes within a first user-specified region while peptide barcodes conjugated to affinity reagents bound to target analytes in a second user-specified region remain conjugated. In some embodiments, peptide barcodes are collected from a first user-specified region and sequenced, then peptide barcodes are collected from a second user-specified region and sequenced. In some embodiments, an image of the sample is captured prior to releasing peptide barcodes. In some embodiments, peptide barcodes released from a first user-specified region are sequenced and the sequencing reads are mapped to a spatial representation (e.g., image) of the sample. In some embodiments, peptide barcodes are iteratively released from multiple user-specified regions within a sample, sequenced, and mapped to a spatial representation of the sample. Accordingly, aspects of the present disclosure relate, in part, to determining the abundance of target analytes within user-specified regions within a sample.

Aspects of the present disclosure relate to the use of an affinity reagent conjugated to a peptide barcode to determine the identity of a target analyte within a sample, wherein the peptide barcode is modified only when the peptide barcode is in the same location as the target analyte. In some embodiments, a peptide barcode is modified by a stimulus that is specific to a cell type or cellular environment. In some embodiments, a peptide barcode is pH-sensitive. In some embodiments, a peptide barcode that is exposed to a low pH is modified. In some embodiments, a peptide barcode that is transported to a cellular lysosome is modified. In some embodiments, modifications on the peptide barcode indicate the peptide barcode was present in an environment that modified the peptide barcode.

Aspects of the present disclosure relate, at least in part, to collecting peptide barcodes that are modified by a cell type-specific stimulus. For example, a peptide barcode can be susceptible to modification by an enzyme that is present in a specific cell type. Methods of expressing an enzyme in a specific cell type are known in the art. For example, an enzyme engineered to modify a peptide barcode can be expressed in a cell type under the control of a cell type-specific promoter.

In some embodiments, an ADC is used as an affinity reagent, for example, in a method of identifying the presence of a target analyte in a sample (e.g., a target analyte to which the antibody component of the ADC binds), as described herein. In some embodiments, an ADC is used as an affinity reagent in a method of identifying a target analyte in a region of a sample, as described herein. It should be appreciated that such methods of the disclosure are not limited to providing information relating to a target analyte in a sample. For example, in some embodiments, a method in which a peptide barcode of an affinity reagent (e.g., an ADC) is indicative of the presence or location of a target analyte in a sample would be further indicative of the presence or location of the affinity reagent (e.g., the ADC) in the sample. In this way, the methods described herein can be used to provide information relating to an affinity reagent, such as the therapeutic effectiveness of an ADC in a subject or a biological sample derived from a subject.

Accordingly, in some aspects, the disclosure provides a method of identifying a therapeutic agent (e.g., an ADC) in a sample. In some embodiments, the term “therapeutic agent” refers to a known therapeutic agent or a putative therapeutic agent. In some embodiments, the method comprises: contacting a sample with a therapeutic agent (e.g., an ADC), where the therapeutic agent is conjugated to a peptide barcode described herein; releasing the peptide barcode from the therapeutic agent in the sample; and sequencing the peptide barcode to identify the therapeutic agent in the sample. In some embodiments, the sequencing comprises identifying the presence of the therapeutic agent in the sample (or a specified region of the sample as described herein). In some embodiments, the sequencing comprises determining a concentration of the therapeutic agent in the sample (or a specified region of the sample as described herein). In some embodiments, the method further comprises evaluating the therapeutic effectiveness of the therapeutic agent. In some embodiments, the therapeutic effectiveness is evaluated based on the presence, concentration, and/or localization of the therapeutic agent in the sample as described herein. In some embodiments, the therapeutic agent comprises an affinity reagent described herein.

In some aspects, the disclosure provides a method of identifying a therapeutic agent (e.g., an ADC) in a region of a sample. In some embodiments, the method comprises: (i) contacting a sample with a therapeutic agent conjugated to a peptide barcode indicative of the therapeutic agent; (ii) releasing the peptide barcode from the therapeutic agent in a first region of the sample; and (iii) sequencing the peptide barcode to identify the therapeutic agent in the first region of the sample. In some embodiments, after step (ii), the peptide barcode remains conjugated to the therapeutic agent in a second region of the sample. In some embodiments, the method further comprises releasing the peptide barcode from the therapeutic agent in the second region of the sample. In some embodiments, the sequencing comprises identifying the therapeutic agent in each of the first and second regions of the sample. In some embodiments, the method further comprises washing the sample between step (i) and step (ii). In some embodiments, the therapeutic agent comprises an affinity reagent described herein.

In some aspects, the disclosure provides a method of identifying an ADC in a sample. In some embodiments, the method comprises: contacting a sample with an ADC, where the ADC is conjugated to a peptide barcode described herein; releasing the peptide barcode from the ADC in the sample; and sequencing the peptide barcode to identify the ADC in the sample. In some embodiments, the sequencing comprises identifying the presence of the ADC in the sample (or a specified region of the sample as described herein). In some embodiments, the sequencing comprises determining a concentration of the ADC in the sample (or a specified region of the sample as described herein). In some embodiments, the method further comprises evaluating the therapeutic effectiveness of the ADC. In some embodiments, the therapeutic effectiveness is evaluated based on the presence, concentration, and/or localization of the ADC in the sample as described herein.

In some aspects, the disclosure provides a method of evaluating an ADC in a subject. In some embodiments, the method comprises: providing a sample of a subject receiving an ADC (e.g., a subject to which the ADC has been administered), where the ADC is conjugated to a peptide barcode described herein; releasing the peptide barcode from the ADC in the sample; and sequencing the peptide barcode to evaluate the ADC in the subject. In some embodiments, the subject is a human. In some embodiments, the subject is a non-human animal (e.g., a non-human mammal). In some embodiments, the sample is a serum sample of the subject. In some embodiments, the sample is a tissue sample of the subject.

In some embodiments, the method further comprises evaluating the therapeutic effectiveness of the ADC in the subject. In some embodiments, the therapeutic effectiveness is evaluated based on the presence, concentration, and/or localization of the ADC in the sample as described herein. In some embodiments, the therapeutic effectiveness is evaluated based on the duration of time between the subject receiving the ADC and the sample being obtained from the subject. In some embodiments, the therapeutic effectiveness is evaluated by comparing sequencing results with one or more other samples of the subject (e.g., a sample obtained prior to treatment with the ADC, a sample obtained at a different time point following treatment with the ADC). In some embodiments, the method further comprises adjusting (e.g., increasing or decreasing) the dosage amount of the ADC in the therapeutic regimen of the subject based on the sequencing or information derived therefrom.

In some embodiments, the method comprises: (i) providing a sample of a subject receiving an ADC, wherein the ADC is conjugated to a peptide barcode; (ii) releasing the peptide barcode from the ADC in the sample; (iii) sequencing the peptide barcode; and (iv) evaluating the ADC based on the sequencing. In some embodiments, the evaluating comprises determining a concentration of the ADC in the sample based on the sequencing. In some embodiments, the evaluating comprises comparing the concentration of the ADC in the sample to a control sample. In some embodiments, the control sample is a sample of the subject prior to receiving the ADC. In some embodiments, the control sample is a sample of the subject at a different time point after receiving the ADC.

In some embodiments, step (ii) comprises: releasing the peptide barcode from the ADC in a first region of the sample, wherein the peptide barcode remains conjugated to the ADC in a second region of the sample. In some embodiments, the method further comprises releasing the peptide barcode from the ADC in the second region of the sample. In some embodiments, the sequencing comprises sequencing the peptide barcode released from the ADC in each of the first and second regions of the sample. In some embodiments, the evaluating comprises: determining a first concentration of the ADC in the first region of the sample based on the sequencing; and determining a second concentration of the ADC in the second region of the sample based on the sequencing.

In some embodiments, the ADC comprises a drug component conjugated to an antibody component via the peptide barcode. In some embodiments, the peptide barcode is conjugated to the ADC via a linker. In some embodiments, the linker comprises a cleavage site. In some embodiments, the peptide barcode is released from the ADC by contacting the linker with a cleaving agent (e.g., a cleaving enzyme, such as an endopeptidase). In some embodiments, the linker is a photocleavable linker. In some embodiments, the peptide barcode is released from the ADC by exposing the linker to photocleaving light (e.g., ultraviolet radiation or a laser). In some embodiments, the sample is a serum sample of the subject. In some embodiments, the sample is a tissue sample of the subject. In some embodiments, the subject is a human. In some embodiments, the subject is a non-human animal.

In some embodiments, the method further comprises, prior to providing the sample: administering the ADC to the subject, where the ADC is conjugated to the peptide barcode; and obtaining the sample of the subject after the administering. In some embodiments, the subject is a non-human animal, and the method comprises: obtaining one or more tissue samples from the subject; and sequencing the one or more tissue samples to evaluate biodistribution of the ADC in the subject.

### Certain Peptide Barcode Implementations

Aspects of the present disclosure relate to the use of the peptide barcodes of the present disclosure to enhance and/or accelerate research. For example, as described herein, the peptide barcodes of the present disclosure can be used for single molecule peptide barcode sequencing. In addition, the peptide barcodes of the present disclosure can be used to accelerate functional screening of proteins. Within the context of screening proteins, antibodies, and engineered enzymes, peptide barcodes can be used to identify proteins with desired characteristics and for orthogonal validation of hits in pooled screens. Within the context of screening protein-protein or protein-drug interactions, peptide barcodes can be used to pinpoint specific amino acid residues involved in protein-protein interactions. Within the context of screening mRNA vaccine candidates for protein production, peptide barcodes can be used to evaluate mRNA translation efficiency directly at the protein level. Within the context of screening lipid nanoparticle (LNP) therapeutic payload delivery efficiency, peptide barcodes can be used to directly confirm the delivery and presence of therapeutic payloads in a specific cell or tissue.

In some embodiments, the peptide barcodes of the present disclosure are used to screen mRNA protein production. In some embodiments, the peptide barcodes of the present disclosure are used to screen mRNA vaccine candidates for protein production. To screen for mRNA protein production, an mRNA can be designed to encode a protein of interest and a peptide barcode. The mRNA can then be delivered to a specific cell and the cell's endogenous translation system can translate the mRNA to produce the protein of interest fused to the peptide barcode. The peptide barcode can be cleaved from the protein of interest using any appropriate cleaving agent (e.g., described herein or known in the art). The peptide barcode can then be collected and sequenced. The presence of sequencing reads associated with a specific barcode is indicative of translation of the protein of interest to which the peptide barcode was fused. This method can be multiplexed to screen mRNA translation efficiency of thousands of mRNA molecules at once. This method can be further modified to screen delivery of mRNA vaccine candidates. An mRNA vaccine candidate encoding a vaccine protein or peptide can be modified to encode a peptide barcode in addition to a vaccine candidate. Presence of sequencing reads associated with a peptide barcode is indicative of production of a vaccine candidate in a specific cell.

In some embodiments, the peptide barcodes of the present disclosure are used to screen payload delivery efficiencies. In some embodiments, a payload is a molecule that is intended to be delivered to a particular cell or tissue. In some embodiments, a payload is a polynucleotide. In some embodiments, a payload is an mRNA molecule. In some embodiments, a payload is fused to a peptide barcode. To evaluate payload delivery efficiency, a peptide barcode fused to the payload can be cleaved from the payload and sequenced. The presence of sequencing reads associated with a peptide barcode is indicative of successful delivery of the payload. In some embodiments, a payload fused to a peptide barcode is delivered by a lipid-based delivery system. In some embodiments, the lipid-based delivery system is a lipid nanoparticle (LNP). An LNP carrying a payload fused to a peptide barcode can be used to deliver the payload to a specific cell. In some embodiments, the payload is an mRNA molecule encoding a payload protein and a peptide barcode. Once an LNP delivers an mRNA molecule encoding a payload protein and a peptide barcode to a specific cell, the cell's endogenous translation system translates the mRNA resulting in a protein payload fused to a peptide barcode. The peptide barcode can then be cleaved from the payload protein and sequenced. The presence of sequencing reads associated with a peptide barcode is indicative of the presence of the payload protein within a specific cell. In some embodiments, a peptide barcode further comprises a cell surface receptor. In some embodiments, a cell surface receptor shuttles a peptide barcode to a cell surface for more efficient collection of peptide barcodes. This screening method can be used to screen delivery methods to identify effective and highly specific delivery vehicles.

### Tags

In some embodiments, a protein of interest described herein is attached to a tag. In some embodiments, an affinity reagent described herein is attached to a tag. In some embodiments, a peptide barcode described herein is attached to a tag. In some embodiments, an endopeptidase described herein is attached to a tag. In some embodiments, a tag is a first tag. In some embodiments, a tag is a second tag. In some embodiments, a tag is a third tag. In some embodiments, a first tag is attached to a second tag. In some embodiments, a first tag is attached to a third tag. In some embodiments, a second tag is attached to a third tag. As used herein, in some embodiments, a tag refers to a segment of amino acids attached to a protein of interest, an affinity reagent, a peptide barcode, or another tag. In some embodiments, a tag is attached to a terminal end (e.g., terminus) of a protein of interest, affinity reagent, peptide barcode, or another tag. In some embodiments, a tag is attached to the C-terminus of an a protein of interest, affinity reagent, peptide barcode, or another tag. In some embodiments, a tag is attached to the N-terminus of a protein of interest, affinity reagent, peptide barcode, or another tag. In some embodiments, a tag is attached to an internal position of a protein of interest, affinity reagent, peptide barcode, or another tag.

In some embodiments, a tag (e.g., a first tag, a second tag, a third tag) comprises at least two amino acids. For example, in some embodiments, a tag comprises at least 2, at least 3, at least 4, at least 5, at least 6, at least 8, at least 10, at least 15, at least 25, at least 30, at least 40, at least 50, at least 60, at least 80, at least 100, or more, amino acids. In some embodiments, a tag comprises between about 2 and about 200 amino acids (e.g., 2-150 amino acids, 2-100 amino acids, 50-200 amino acids, 50-150 amino acids, 50-100 amino acids, 4-80 amino acids, 5-50 amino acids, 5-30 amino acids, 5-20 amino acids, 10-100 amino acids, 20-80 amino acids, 30-70 amino acids).

In some embodiments, a tag comprises one or more functional components. For example, in some embodiments, a tag comprises one or more of an affinity tag (e.g., a polyhistidine tag), a modification tag (e.g., a biotinylation tag), a solubility tag (e.g., small ubiquitin-like modifier (SUMO) tag), a linker, and a cleavage site for a protease (e.g., an endopeptidase cleavage site).

In some embodiments, a tag comprises a polyhistidine-tag. In some embodiments, a polyhistidine-tag comprises a segment of two or more histidine amino acids. In some embodiments, a polyhistidine-tag comprises a segment of between about 2 and about 15 (e.g., 4, 6, 8, 10, 12, 14, 15) histidine amino acids. In some embodiments, a polyhistidine-tag comprises a hexahistidine-tag (e.g., 6× His-tag). In some embodiments, a polyhistidine-tag comprises a decahistidine-tag (e.g., 10× His-tag). In some embodiments, a tag comprises two or more (e.g., two, three, four) polyhistidine-tags.

In some embodiments, a tag comprises a biotinylation tag. In some embodiments, a biotinylation tag comprises at least one biotin ligase recognition sequence. In some embodiments, a biotinylation tag comprises two biotin ligase recognition sequences oriented in tandem. In some embodiments, a biotin ligase recognition sequence refers to an amino acid sequence that can be recognized by a biotin ligase, which catalyzes a covalent linkage between the sequence and a biotin molecule. In some embodiments, a biotin ligase recognition sequence comprises an amino acid sequence of SEQ ID NO: 36. In some embodiments, a tag comprises two or more (e.g., two, three, four) biotin ligase recognition sequences.

In some embodiments, a tag comprises at least one polyhistidine-tag and at least one biotin ligase recognition sequence. In some embodiments, a tag comprises at least one polyhistidine-tag and at least two biotin ligase recognition sequences. In some embodiments, a tag comprises at least two polyhistidine-tags and at least one biotin ligase recognition sequence.

In some embodiments, a tag comprises a solubility tag. In some embodiments, a tag comprises a tag peptide or tag protein. Examples of tag peptides include, without limitation, calmodulin-binding peptide (CBP) tag, FLAG epitope, human influenza hemagglutinin (HA) tag, Myc epitope, streptavidin-binding peptide, Strep tag, Strep-II tag, intrinsically disordered tag, Fasciola hepatica 8-kDa antigen (Fh8), maltose-binding protein (MBP), N-utilization substance (NusA), thioredoxin (Trx), small ubiquitin-like modifier (SUMO), glutathione-S-transferase (GST), solubility-enhancer peptide sequences (SET), IgG domain B1 of Protein G (GB1), IgG repeat domain ZZ of Protein A (ZZ), mutated dehalogenase (HaloTag), Solubility eNhancing Ubiquitous Tag (SNUT), seventeen kilodalton protein (Skp), bacteriophage V5 epitope, phage T7 protein kinase (T7PK), E. coli secreted protein A (EspA), monomeric bacteriophage T7 0.3 protein (Orc protein; Mocr), E. coli trypsin inhibitor (Ecotin), calcium-binding protein (CaBP), stress-responsive arsenate reductase (ArsC), N-terminal fragment of translation initiation factor IF2 (IF2-domain I), stress-responsive proteins (e.g., RpoA, SlyD, Tsf, RpoS, PotD, Crr), and E. coli acidic proteins (e.g., msyB, yjgD, rpoD). Additional examples of tags are known in the art and may be used in accordance with the disclosure. See, e.g., Costa, S., et al. “Fusion tags for protein solubility, purification and immunogenicity in Escherichia coli: the novel Fh8 system.” Front Microbiol. 2014 Feb. 19; 5:63; and Kimple, M. E., et al. “Overview of Affinity Tags for Protein Purification.” Curr Protoc Protein Sci. 2013, 73:Unit 9-9, the relevant contents of which are incorporated by reference herein.

In some embodiments, a tag comprises a linker. For example, in some embodiments, a tag comprises a polyhistidine-tag and a linker between the polyhistidine-tag and an amino acid of a proteinaceous molecule described herein (e.g., a protein of interest, an affinity reagent, a peptide barcode, or an endopeptidase, such as an aminopeptidase). In some embodiments, a tag comprises a biotin ligase recognition sequence and a linker between the biotin ligase recognition sequence and an amino acid of a proteinaceous molecule described herein (e.g., a protein of interest, an affinity reagent, a peptide barcode, or an endopeptidase, such as an aminopeptidase). In some embodiments, a tag comprises a biotin ligase recognition sequence, a polyhistidine-tag, and a linker between the biotin ligase recognition sequence and the polyhistidine-tag. In some embodiments, a tag comprises two biotin ligase recognition sequences and a linker between the two biotin ligase recognition sequences.

In some embodiments, a linker of a tag comprises one or more amino acids. In some embodiments, a linker comprises at least 2, at least 3, at least 4, at least 5, at least 6, at least 8, at least 10, at least 12, at least 15, at least 30, at least 25, at least 30, or more, amino acids. In some embodiments, a linker comprises between about 1 and about 50 amino acids (e.g., 1-50 amino acids, 1-30 amino acids, 2-25 amino acids, 3-30 amino acids, 6-25 amino acids, 2-15 amino acids, 1-12 amino acids).

In some embodiments, a linker of a tag comprises at least one glycine amino acid. In some embodiments, a linker comprises at least one glycine-serine (GS) motif. In some embodiments, a linker comprises an amino acid sequence of the following formula: (GₘS)ₙ(SEQ ID NO: 1), where: G is glycine; S is serine; m is an integer from 1 to 5, inclusive; and n is an integer from 1 to 6, inclusive. In some embodiments, m is an integer from 1 to 3, inclusive. In some embodiments, m is 2 or 3. In some embodiments, m is 2. In some embodiments, m is 3. In some embodiments, n is an integer from 1 to 4, inclusive. In some embodiments, n is an integer from 1 to 3, inclusive. In some embodiments, n is 1 or 3. In some embodiments, n is 1. In some embodiments, n is 3.

In some embodiments, a tag comprises or is attached to a cleavage site for a protease (e.g., an endopeptidase cleavage site).

In some embodiments, a tag comprises an amino acid sequence selected from Table 1. In some embodiments, a tag comprises an amino acid sequence that is at least 40% identical to an amino acid sequence selected from Table 1. In some embodiments, a tag has at least 45%, at least 50%, at least 60%, at least 70%, at least 80%, at least 85%, at least 90%, at least 92%, at least 94%, at least 95%, at least 96%, at least 98%, or higher, amino acid sequence identity to an amino acid sequence selected from Table 1. In some embodiments, a tag has 25-50%, 50-60%, 60-70%, 70-80%, 80-90%, 90-95%, 92-99%, 94-99%, 95-99%, 40-100%, 50-100%, 60-100%, 70-100%, 80-100%, 90-100%, 92-100%, 94-100%, 95-100%, 96-100%, or 100% amino acid sequence identity to an amino acid sequence selected from Table 1.

Accordingly, in some embodiments, a protein construct of the disclosure comprises a tag, wherein the tag has an amino acid sequence selected from Table 1. In some embodiments, the protein construct comprises a protein of interest or an affinity reagent, and at least one tag, wherein the tag has an amino acid sequence that is at least 40% identical to an amino acid sequence selected from Table 1.

In some aspects, the disclosure provides a single polypeptide comprising a protein of interest or an affinity reagent described herein attached to a tag described herein or known in the art. For example, in some embodiments, aspects of the disclosure relate to the use of a fusion polypeptide of a protein of interest or an affinity reagent fused to a tag. In some embodiments, a fusion polypeptide comprises a tag fused to a terminal end of the protein of interest or affinity reagent. In some embodiments, a fusion polypeptide comprises a tag fused to the C-terminal end of the protein of interest or affinity reagent. In some embodiments, a fusion polypeptide comprises a tag fused to the N-terminal end of the protein of interest or affinity reagent. In some aspects, the disclosure provides a nucleic acid encoding a fusion polypeptide described herein. In some embodiments, the nucleic acid is an expression construct encoding a fusion polypeptide of a protein of interest or an affinity reagent fused to a tag.

### Polypeptide Analysis

In some aspects, the disclosure provides methods of polypeptide analysis (e.g., polypeptide sequencing). In some embodiments, a method of polypeptide analysis comprises: contacting a polypeptide with one or more amino acid binding proteins; monitoring a signal for signal pulses corresponding to interactions between the one or more amino acid binding proteins and the polypeptide; and determining at least one chemical characteristic of the polypeptide based on a characteristic pattern in the signal.

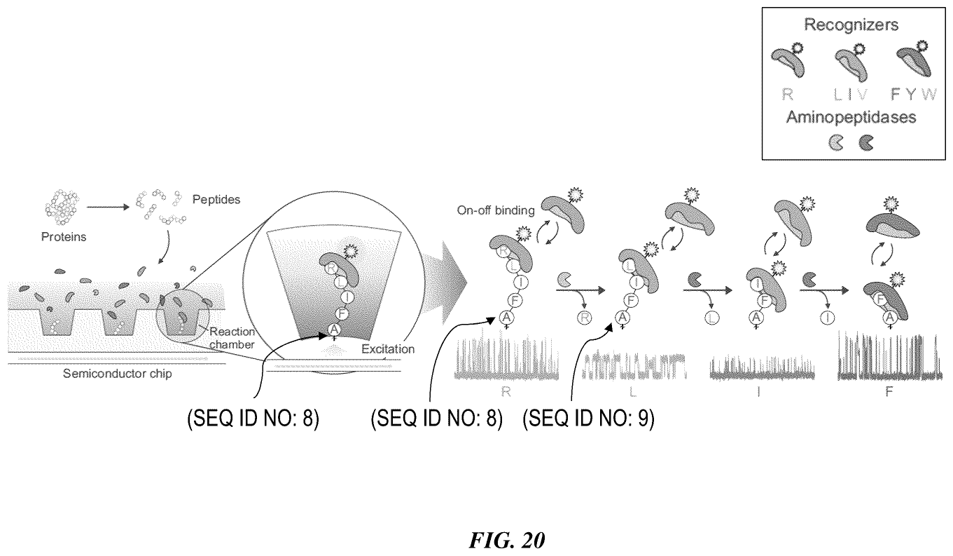

A non-limiting example of polypeptide structure analysis by detecting single molecule binding interactions during a polypeptide degradation process is illustrated in FIG. 20. An example signal trace is shown depicting different association (e.g., binding) events at times corresponding to changes in the signal. As shown, an association event between an amino acid recognizer and a terminal end of a polypeptide produces a change in magnitude of the signal that persists for a duration of time. Different association events are illustrated for different amino acids exposed at the terminal end of the polypeptide. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage in the context of a polypeptide sequencing reaction refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, as shown in FIG. 20, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIG. 20, in some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the disclosure are provided herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the disclosure are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor polypeptide sequencing reactions in accordance with the disclosure. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N or C terminus) of a polypeptide is immobilized and the other terminus (e.g., the C or N terminus) is analyzed as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

In some embodiments, a polypeptide sequencing reaction is performed under conditions in which recognition and cleavage of amino acids can occur in a single reaction mixture. In some embodiments, the polypeptide sequencing reaction is performed in a reaction mixture comprising one or more reagents suitable for single-molecule analytical methods. In some embodiments, a polypeptide sequencing reaction is performed in a reaction mixture comprising an oxygen scavenger (e.g., a free-radical scavenger, a triplet state quencher). In some embodiments, oxygen scavengers can be useful to mitigate the presence of reactive oxygen species (ROS), such as hydrogen peroxide, hydroxyl radical, nitric oxide, peroxyl radical, peroxynitrite anion, singlet oxygen, and superoxide anion. See, e.g., International Publication No. WO 2021/236983 A2, describing the use of oxygen-scavenging systems in polypeptide sequencing reactions, the disclosure of which is incorporated herein by reference in entirety.

Suitable oxygen scavengers for sequencing reactions described herein include, without limitation, glucose (e.g., β-D-glucose), pyruvate (e.g., sodium pyruvate) (see, e.g., Acc Chem Res. 2021 Apr. 6; 54(7):1779-1790; and Talanta 2010 Jun. 15; 81(4-5):1840-6), protocatechuic acid (PCA), N,N′-dimethylthiourea, Mannitol (see, e.g., J Biol Chem. 2007 Oct. 19; 282(42):30452-65), DMSO, carboxy-PTIO (see, e.g., Nitric Oxide 2006 September; 15(2):163-76), Trolox (6-hydroxy-2,5,7,8-tetramethylchroman-2-carboxylic acid), α-tocopherol (see, e.g., Biochim Biophys Acta. 2004 Mar. 22; 1636(2-3):136-50), Ebselen (2-phenyl-1,2-benzisoselenazol-3(2H)-one) (see, e.g., J Biol Chem. 2007 Oct. 19; 282(42):30452-65), uric acid (see, e.g., J Biol Chem. 2004 Feb. 6; 279(6):4425-32.), sodium azide (see, e.g., J Biol Chem. 2007 Oct. 19; 282(42):30452-65), MnTBAP (manganese(III)-tetrakis(4-benzoic acid)porphyrin) (see, e.g., Bioorg Med Chem. 2002 September; 10(9):3013-21), and Tiron (4,5-dihydroxybenzene-1,3-disulfonate) (see, e.g., Circ Res. 2004 Jan. 9; 94(1):37-45).

In some embodiments, a polypeptide sequencing reaction is performed in a reaction mixture comprising an oxygen-scavenging system, which can include one or more oxygen scavengers described herein and one or more catalysts (e.g., enzymes). Examples of oxygen-scavenging systems are known in the art and include, for example, glucose oxidase and catalase (GOC) systems and protocatechuic acid (PCA)/protocatechuate-3,4-dioxygenase (PCD) systems (see, e.g., Biophys J. 2008 Mar. 1; 94(5): 1826-1835; and ACS Nano. 2012 Jul. 24; 6(7): 6364-6369). See also, e.g., International Publication No. WO 2021/236983 A2, describing polypeptide sequencing reactions using PCA/PCD or pyranose oxidase/catalase oxygen-scavenging systems. In some embodiments, the oxygen-scavenging system comprises one or more oxygen scavengers described herein and one or more enzymes (e.g., glucose oxidase, catalase, and/or dioxygenase).

### Devices and Systems

Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light source external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The integrated device may include an optical system for receiving excitation light and directing the excitation light among the sample well array. The optical system may include one or more grating couplers configured to couple excitation light to other optical components of the integrated device and direct the excitation light to the other optical components. For example, the optical system may include optical components that direct the excitation light from the grating coupler(s) towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a sample well and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an integrated device may be configured to perform single-molecule analysis in combination with an instrument as described above. It should be appreciated that the integrated device described herein is intended to be illustrative and that other integrated device configurations may be configured to perform any or all techniques described herein.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel 1-112 may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel 1-112 (e.g., in photodetection region PD) responsive to excitation photons from the light source. For instance, the excitation photons may arrive at pixel 1-112 prior to the arrival of fluorescence emission photons from the sample well. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### SEQUENCE INFORMATION

As described herein, in some embodiments, a tag of the disclosure can comprise an amino acid sequence provided herein or known in the art. In some embodiments, a tag comprises an amino acid sequence that shares a percentage of sequence identity with an amino acid sequence selected from Table 1. For the purposes of comparing two or more amino acid sequences, the percentage of “sequence identity” between a first amino acid sequence and a second amino acid sequence (also referred to herein as “amino acid identity”) may be calculated by: dividing [the number of amino acid residues in the first amino acid sequence that are identical to the amino acid residues at the corresponding positions in the second amino acid sequence] by [the total number of amino acid residues in the first amino acid sequence] and multiplying by [100], in which each deletion, insertion, substitution or addition of an amino acid residue in the second amino acid sequence compared to the first amino acid sequence is considered as a difference at a single amino acid residue (position).

Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Additionally, or alternatively, two or more sequences may be assessed for the identity between the sequences. The terms “identical” or percent “identity” in the context of two or more amino acid sequences, refer to two or more sequences or subsequences that are the same. Two sequences are “substantially identical” if two sequences have a specified percentage of amino acid residues that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using one of the above sequence comparison algorithms or by manual alignment and visual inspection. Optionally, the identity exists over a region that is at least about 25, 50, 75, or 100 amino acids in length, or over a region that is 100 to 150, 150 to 200, 100 to 200, or 200 or more, amino acids in length.

Additionally, or alternatively, two or more sequences may be assessed for the alignment between the sequences. The terms “alignment” or “percent alignment” in the context of two or more amino acid sequences, refer to two or more sequences or subsequences that are the same. Two sequences are “substantially aligned” if two sequences have a specified percentage of amino acid residues that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the above sequence comparison algorithms or by manual alignment and visual inspection. Optionally, the alignment exists over a region that is at least about 25, 50, 75, or 100 amino acids in length, or over a region that is 100 to 150, 150 to 200, 100 to 200, or 200 or more amino acids in length.

TABLE 1
Non-limiting examples of tag sequences.

| | | SEQ |
|---|---|---|
| | | ID |
| Description | Sequence | NO: |
| 6x His-tag | HHHHHH | 32 |
| 6x His-tag with linker | GGSHHHHHH | 33 |
| 10x His-tag | HHHHHHHHHH | 34 |
| 10x His-tag with | GHHHHHHHHHH | 35 |
| linker | | |
| Biotinylation tag | GLNDFFEAQKIEWHE | 36 |
| Biotinylation tag with | GGGSGLNDFFEAQKIEWHE | 37 |
| linker | | |
| Biotinylation tag with | GGGSGGGSGGGSGLNDFFEAQKIEWHE | 38 |
| linker | | |
| Bis-biotinylation tag | GGGSGGGSGGGSGLNDFFEAQKIEWHEGGGSGGGSGGGSGLNDFFEAQKIE | 39 |
| with linkers | WHE | |
| Bis-biotinylation tag | GSGGGSGGGSGGGSGLNDFFEAQKIEWHEGGGSGGGSGGGSGLNDFFEAQK | 40 |
| with linkers | IEWHE | |
| His/biotinylation tags | GHHHHHHHHHHGGGSGGGSGGGSGLNDFFEAQKIEWHE | 41 |
| with linkers | | |
| His/bis-biotinylation | GHHHHHHHHHHGGGSGGGSGGGSGLNDFFEAQKIEWHEGGGSGGGSGGGSG | 42 |
| tags with linkers | LNDFFEAQKIEWHE | |
| His/bis-biotinylation | GGSHHHHHHHHHHGGGSGGGSGGGSGLNDFFEAQKIEWHEGGGSGGGSGGG | 43 |
| tags with linkers | SGLNDFFEAQKIEWHE | |
| His/bis-biotinylation | GSHHHHHHHHHHGGGSGGGSGGGSGLNDFFEAQKIEWHEGGGSGGGSGGGS | 44 |
| tags with linkers | GLNDFFEAQKIEWHE | |
| Bis-biotinylation/His | GGGGGGSGGGSGLNDFFEAQKIEWHEGGGSGGGSGGGSGLNDFFEAQKIE | 45 |
| tags with linkers | WHEGHHHHHH | |
| Sortase recognition | LPETGG | 46 |
| sequence | | |

### EXAMPLES

### Example 1. Protein Quantification Via Peptide Barcodes

Direct quantification of large panels of proteins in complex samples remains challenging. This example describes a new approach for distinguishing different proteins in a sample or different variants of a protein in a library based on single-molecule peptide sequencing and “peptide barcodes”: unique identifiers or tags. A set of peptide barcodes are introduced which are designed for: 1) optimal performance on a sequencing instrument; 2) resistance to noise; and 3) optimal biochemical compatibility (e.g., solubility, charge). These barcodes can be genetically appended to proteins of interest, for example, using any appropriate methodology as determined by a skilled person based on knowledge in the art (e.g., using synthetic biology methodologies, directed evolution experiments, in vivo methodologies). The composition of the complex sample containing the barcoded proteins can then be assessed at different time points: peptide barcodes are cleaved and sequenced on a sequencing instrument, with the relative amount of each barcode reporting the concentration of the associated protein.

One of the many applications of this approach is screening libraries of single-domain antibodies, for example nanobodies (VHHs). The selection of nanobodies can be performed by ribosome or phage display, which rely on a physical coupling between the genotype (e.g., the DNA or RNA molecules that are used for identification and quantification) and the phenotype (e.g., the protein itself). However, the ribosome or the phage particles are larger and often more soluble than the VHH itself, which can result in oligomerizing or non-soluble VHH variants being retained in the selection. Peptide barcode methodologies described herein can be used to screen VHH libraries directly without requiring genotype/phenotype linkage, as each VHH variant is associated to one or multiple barcodes. The barcoded library is then sequenced before and after selection, and the performance for each VHH variant is assessed by the change in frequency of the associated barcode. This approach is demonstrated, as described below, in a model selection of a GFP-binding nanobody versus an MBP-binding nanobody against beads displaying GFP. This approach can be applied to any protein (or peptide) with binding activity and similarly applied to enzymes.

### Peptide Barcodes: Design and Performance

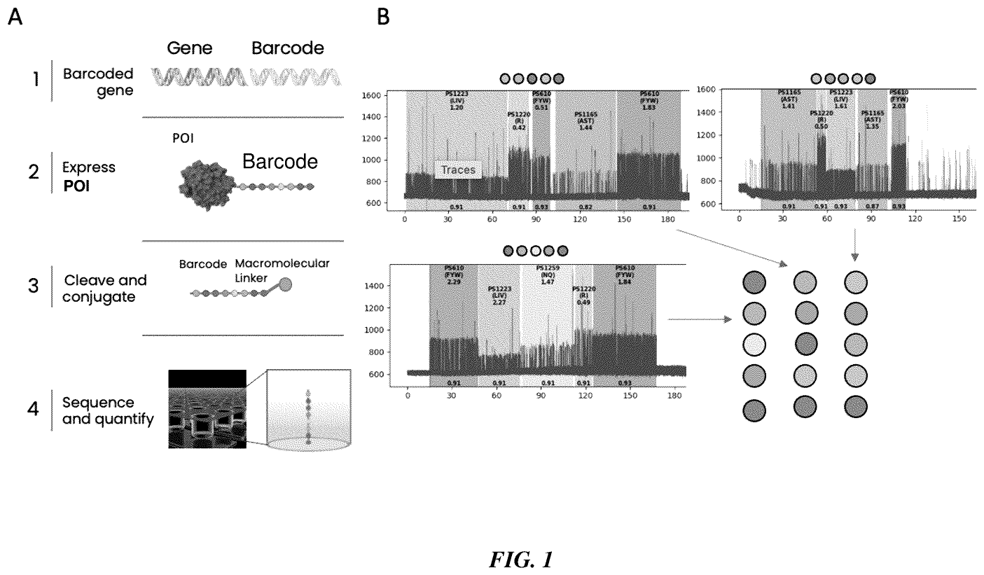

In certain implementations, peptide barcodes are expressed as a tag at the C-terminus of proteins of interest and, prior to sequencing, cleaved by proteolytic methods. Barcodes are then conjugated to macromolecular linkers using an appropriate library prep methodology and sequenced on a sequencing instrument using the real-time dynamic sequencing workflow as previously described. Briefly, the barcodes are captured on a semiconductor chip with exposed N-termini for sequencing. Dye-labeled recognizers bind on and off to N-terminal amino acids (NAAs), generating pulsing patterns with characteristic fluorescence and kinetic properties. Regions corresponding to NAA recognition are termed recognition segments (RSs). Aminopeptidases in solution sequentially remove individual NAAs to expose subsequent amino acids for recognition. Fluorescence lifetime, intensity, and kinetic data are collected in real time and analyzed to determine amino acid sequence. The sequencing profiles of barcodes can be visualized as kinetic signature plots, which are simplified trace-like representations of the time course of complete peptide sequencing containing the median pulse duration (PD) for each RS. See FIG. 1.

Barcodes are designed to fully exploit the powerful sequencing capabilities of a sequencing instrument and provide a distinctive, noise-resistant, signal. The diversity of protein sequence space is such that, using only 5 amino acids (shorter than most common protein tags), multiple sets of hundreds of error-tolerant barcodes can be generated. The chosen barcode sequences can be further optimized by in silico screens for desired properties (e.g., hydrophobicity (computed by SSRC), charge, and isoelectric point).

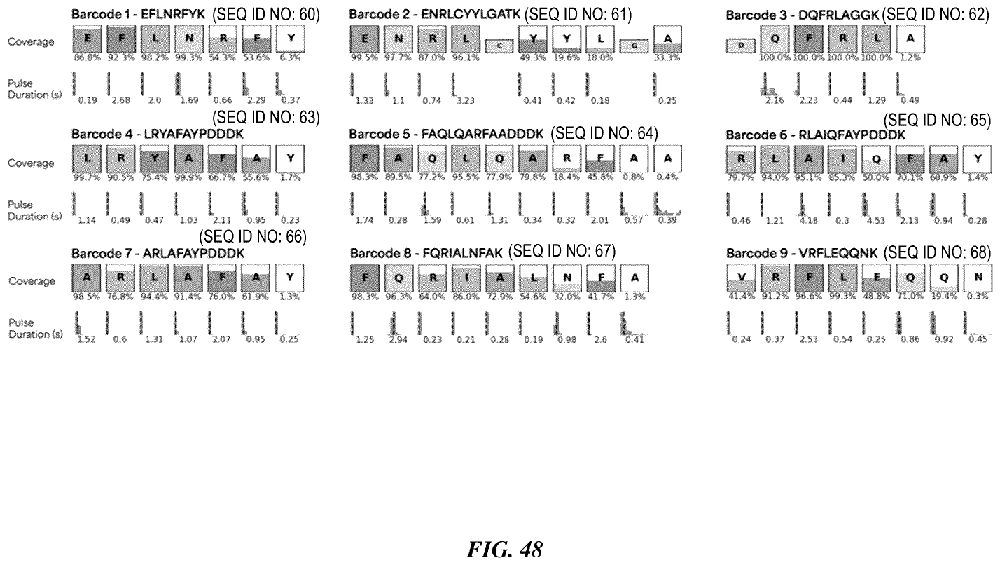

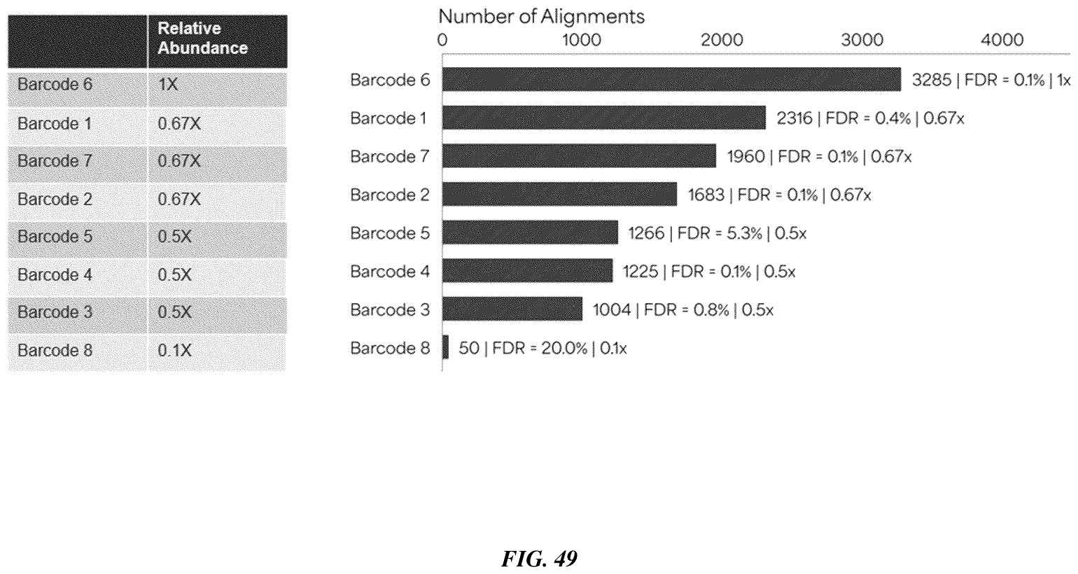

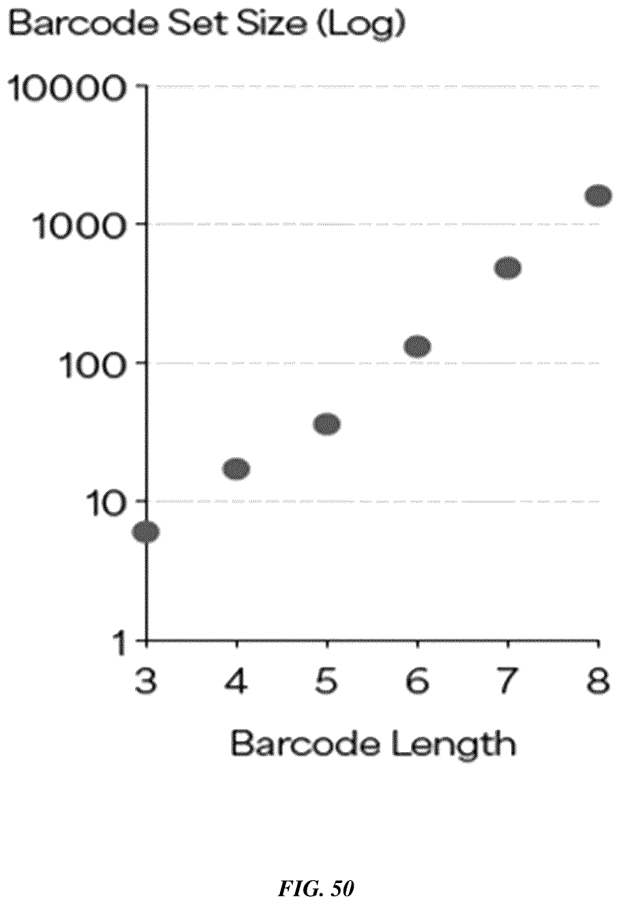

For example, as shown in FIG. 48, the peptide barcodes of the present disclosure are designed to be highly sequenceable. Each amino acid within a peptide barcode can be identified based on its distinct kinetic signature. Because the peptide barcodes of the present disclosure are highly sequenceable, individual peptide barcodes can be identified in a mixture of peptide barcodes (FIG. 49). As shown in FIG. 49, barcodes were prepared individually and pooled into a sample at various abundances. The peptide barcodes were then sequenced to generate unique alignments. The sequencing data generated from each peptide barcode indicated that each peptide barcode was present in the mixture at the expected abundance, demonstrating that individual peptide barcodes can be identified in a mixture of peptide barcodes. The ability to identify a single peptide barcode in a mixture of peptide barcodes indicates the scalability of the peptide barcodes of the present disclosure. The highly distinguishable kinetic signature for each amino acid enables creation of highly complex libraries as compared to traditional sequencing (FIG. 50). Importantly, the use of short barcodes, which are still distinguishable in a mixture of peptide barcodes, minimizes the impact of the peptide barcodes on biological function in an in vitro or in vivo setting.

### Peptide Barcodes Report the Differential Enrichment of Model Nanobody Libraries

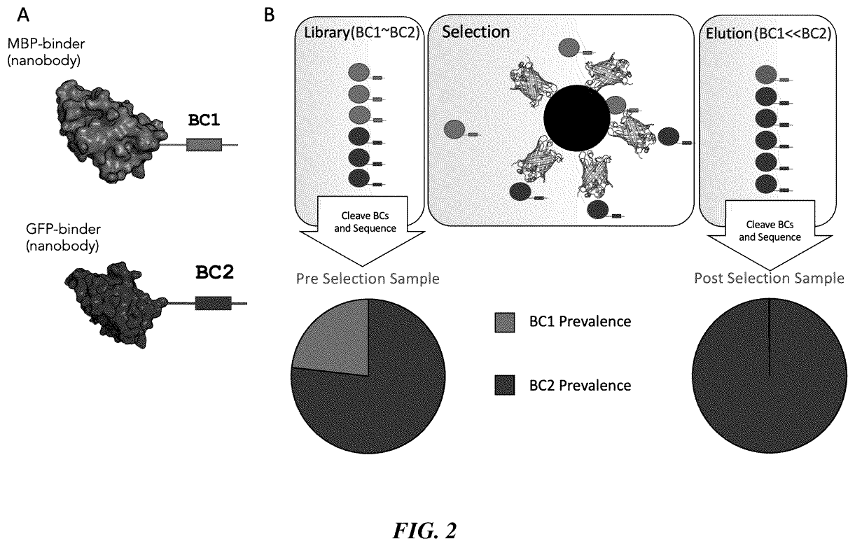

To test the barcodes in a real-world application, a selection was performed on a model library composed of two previously characterized nanobodies, NB1 and NB2. NB1 targets the Maltose Binding Protein (MBP) with high affinity and specificity while NB2 similarly targets the Green Fluorescent Protein (GFP). Genes coding for the two nanobodies were fused with two different C-terminal barcodes, BC1 and BC2, designed for optimal decoding and performance. The barcoded nanobodies were then overexpressed in E. coli, purified, and conjugated at the C-terminus with the macromolecular linker, so that, after proteolysis the barcodes could directly be loaded on the semiconductor chip. One fraction of this library was then stored for further analysis (Pre-Selection Sample). The barcoded model Nanobody Library (BNL) was then subjected to selection by affinity purification on magnetic beads displaying GFP on their surface. The BNL (10 nM) was incubated with the targets for 5 minutes at room temperature, after which the beads were pelleted and washed five times with clean buffer. Barcodes associated with the nanobodies that remained on the beads after washing were then harvested by proteolysis, resulting in a nanomolar barcode solution that was directly loaded on the left-hand half of a semiconductor chip (Post-Selection Sample). Similarly, barcodes from the Pre-Selection Sample were cleaved by proteolysis and captured on the right-hand half of a semiconductor chip. Both the Pre- and Post-Selection Samples were sequenced using a sequencing instrument followed by sequencing data analysis to quantify the prevalence of BC1 vs BC2 in the two samples. The results demonstrated that, upon selection, BC2 (associated with the GFP binder) was enriched ˜3,000× over BC1 (associated with the MBP binder). This approach was thus able to measure the differential enrichment of nanobody clones in a library. See FIG. 2.

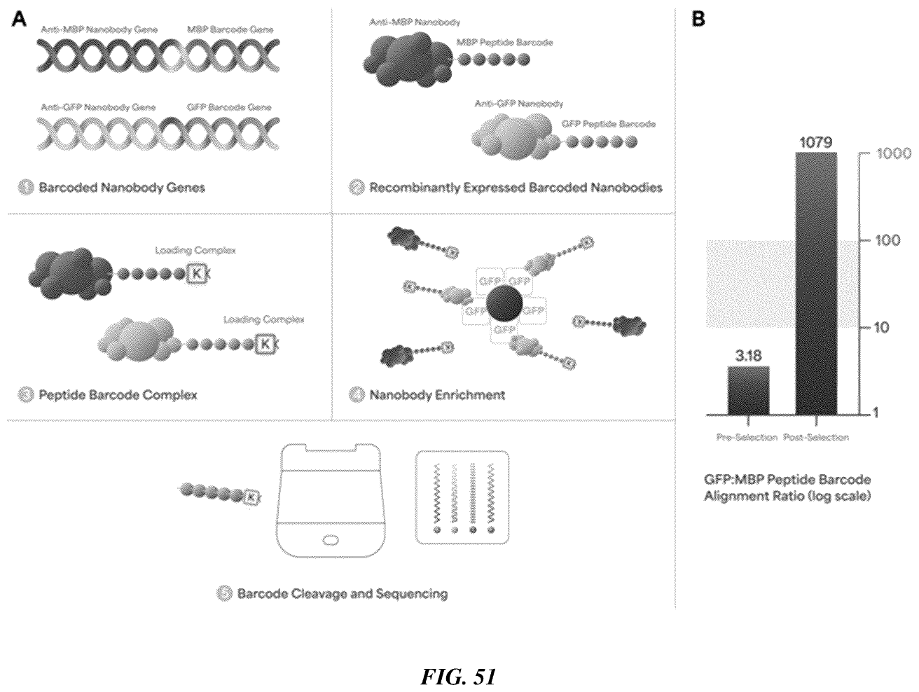

In another set of experiments, anti-GFP and anti-MBP nanobody genes were expressed recombinantly with unique peptide barcodes (FIG. 51, Panels A1-A3). Nanobodies were enriched using GFP immobilized on magnetic beads (FIG. 51, Panel A4). Peptide barcodes were then cleaved and sequenced (FIG. 51, Panel A5). Using this approach, peptide barcode quantification showed a >300-fold enrichment for GFP nanobodies post-selection (FIG. 51, Panel B).

### Example 2. Enzymatic Library Preparation for Peptide Sequencing

Peptide immobilization for sequencing can be achieved via the covalent linkage of peptides to a loading complex which binds specifically to a flow cell. In certain implementations, this can be achieved via the chemical conjugation of a reactive azide (N₃) group to the amine group of a C-terminal lysine. Using copper-free click chemistry, peptides can then be covalently attached to the ‘loading complex’. This method functionalizes all peptides in the sample with a C-terminal Lysine.

A methodology was developed to specifically conjugate the barcode peptide to the loading complex, possibly fishing it out of a mixture of other peptides, and sequence only the barcode peptides on a sequencing instrument. In this methodology, the C-terminus of a protein of interest is tagged with a proteolysis site, barcode, and Sortase A recognition motif (e.g., having the amino acid sequence: LPETGG (SEQ ID NO: 46), among other examples). This tag permits: 1) functionalization and copper-free click chemistry of the barcode peptide to the loading complex, and 2) proteolysis of the N-terminus of the barcode to make it available for Peptide sequencing on a sequencing instrument.

This methodology allows for the generation of peptide sequencing libraries targeted specifically to barcode peptides. In addition, peptide sequencing libraries attached to the protein of interest can be generated, which can then be manipulated according to experimental requirements, and then the barcode released for sequencing by proteolysis.

For example, methods described herein include a method to enzymatically functionalize the terminus of a polypeptide sequence to prepare it for peptide sequencing. Peptide sequencing libraries can be prepared prior to removal of the protein of interest (POI), allowing direct manipulation of the library followed by peptide sequencing (Implementation 1, described below). POIs may also be further characterized on a sequencing instrument, in conjunction with peptide sequencing (Implementation 2, described below).

As described herein, peptide barcoding can be achieved by a method that genetically encodes both a protein of interest and a peptide barcode in a nucleic acid sequence such that when translated into a polypeptide, the protein of interest will be labelled with the peptide barcode in the same polypeptide chain. The method can, in certain implementations, involve the addition of several components to be added to the POI, such as 1) a barcode sequence, 2) a proteolysis sequence to reveal the barcode for peptide sequencing, and 3) an affinity tag to purify loading complexes which are coupled to barcodes (e.g., polypeptide sequences which may be engineered for optimal sequencing on a sequencing instrument). In such methods of protein barcoding, nucleic acid sequences encoding proteins of interest fused to a barcode will be assembled. The POIs are then expressed as libraries or as individual proteins and manipulated according to experimental requirements, including, for example, in vitro POI testing such as antibody characterization, in vivo experimentation in cells or organisms to quantify, track, or characterize the behavior of proteins of interest in cell biology or animal models, ex vivo tissue experimentation. Two examples with accompanying data are described below which illustrate the utility of this approach.

### Implementation 1) Quantification of a Mixture of Antibodies to Measure Binding to Given Target Molecule.

Experiments of this nature are technically feasible by other means, such methods are well known in field of antibody directed evolution, such as phage display, ribosome display or mRNA display. However conceptually each of these techniques relies on the linkage of the polypeptide to the nucleic acid sequence which encodes it, linking phenotype (functional behavior of the protein) to genotype (DNA sequence). Importantly, using the peptide barcoding sequence described herein, this requirement is removed, requiring only a single polypeptide. The correspondence between genotype and phenotype is maintained in silico using computational means (i.e. data files).

### Implementation 2) Parallelized Determination of Biophysical Properties at the Single Molecule Level.

Antibody characterization has been performed by using techniques such as Surface plasmon resonance (SPR), which are ‘bulk’ reactions that must be performed in individual reaction chambers (1 reaction per antibody). Antibodies must be expressed and purified individually in order to be evaluated by this technique. Experimental data described herein demonstrated that the binding properties of individual antibodies can be determined from a complex mixture of barcoded antibodies. First, binding properties were evaluated at the single molecule level by observing the pulsing behavior of antibodies binding to their respective target (in this instance, GFP) in solution. Following this step, corresponding barcodes were revealed by proteolysis directly on a sequencing instrument. Individual barcodes were then sequenced and the identity of each nanobody revealed. This method is scalable according to the ‘barcode space’ (the number of available barcode sequences).

### Method

In one iteration of this method, a POI fused by genetic means to a peptide barcode and Sortase A recognition sequence are expressed and purified using standard means. A Sortase A reaction consisting of a Sortase A reaction buffer, Sortase A enzyme, GlyGlyGly-PEG3-picolyl azide molecule and the POI is incubated for 1 hour at 37° C. Following this reaction, the POI is purified by coupling it to Halo tag protein purification beads. The POI is washed to remove excess azide. A copper-free click chemistry reaction, consisting of the POI conjugated to magnetic beads, a click chemistry reaction buffer, and the loading complex from a protein sequencing kit is incubated for ˜16 hours. After this reaction, excess loading complex is removed by washing the magnetic beads. The POI-Barcode-Loading complex is then released by proteolysis using the TEV protease. The barcode can then be further processed by proteolysis using the UlpI protease, which reveals the first amino acid of the barcode for sequencing on a sequencing instrument.

In another iteration of this method, a POI-Barcode fusion protein is conjugated to an azide molecule in a Sortase A reaction. The Sortase A reaction may be quenched using EDTA if required. Next, using an affinity handle (e.g. his tag, FLAG tag, c-myc tag, GFP tag, SNAP tag, Halo tag, the POI itself, or any other tag and associated enrichment strategy), the POI-Barcode-Azide construct is purified to remove excess azide. A Click chemistry reaction including the loading complex from a protein sequencing kit and the POI-Barcode-Azide conjugates the POI to the loading complex. The barcode is then processed for peptide sequencing by proteolysis.

In a further iteration, the nucleophile used in the Sortase A reaction can be substituted for alternative molecules, including a synthetic polypeptide GlyGlyGlyLys(N₃) (SEQ ID NO: 7), or 3-Azido-1-propylamine, or hydrazide derivatives.

In yet a further iteration, a novel loading complex is generated which, rather than displaying a Click chemistry handle, displays a Sortase A nucleophile (for example an oligoglycine). This permits a workflow in which a Sortase A reaction constituting Sortase A reaction buffer, the recombinant POI-Barcode, Sortase A, and the GGG-Loading complex is used to conjugate the barcode directly to the peptide sequencing complex.

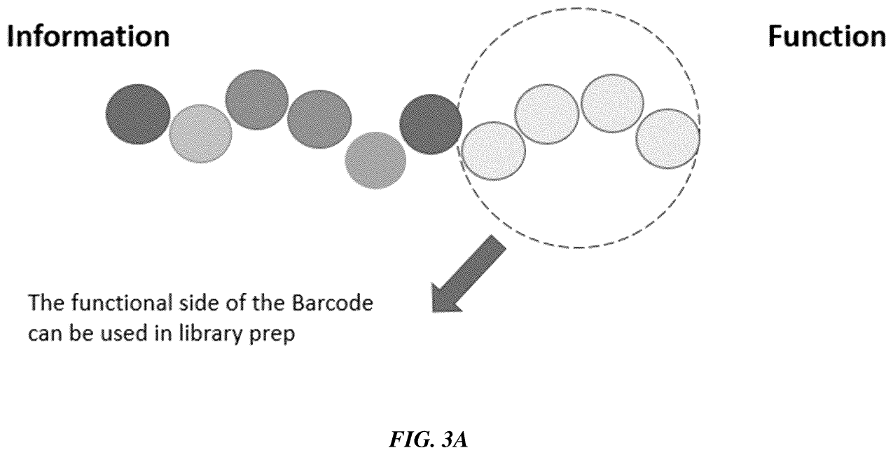

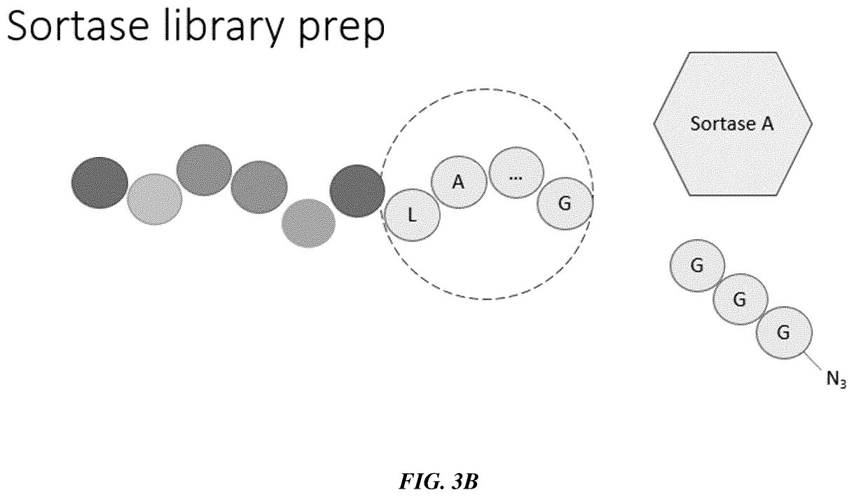

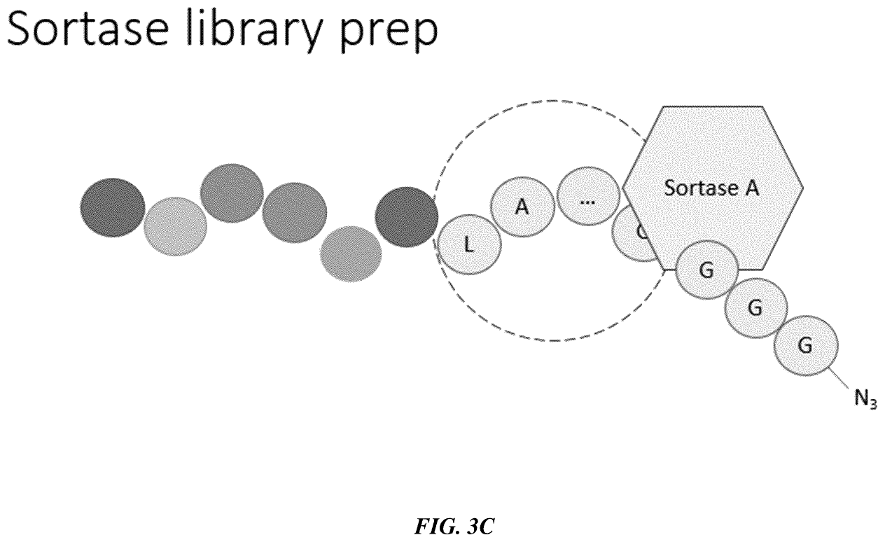

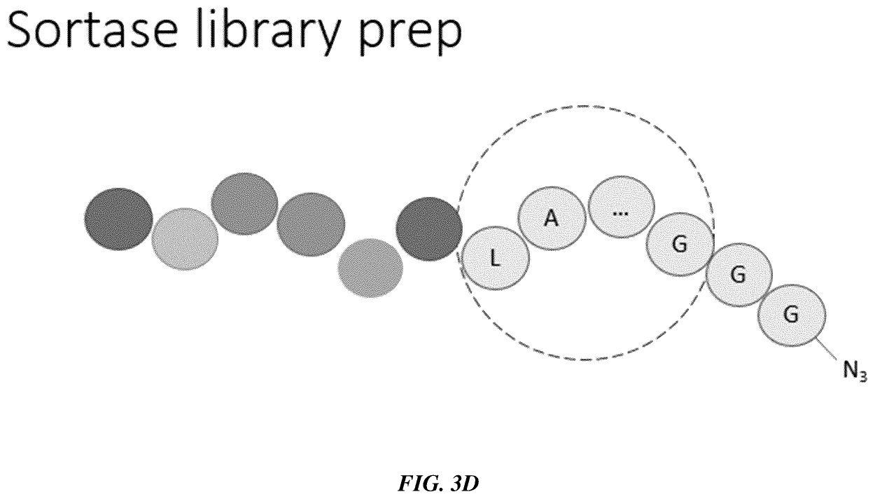

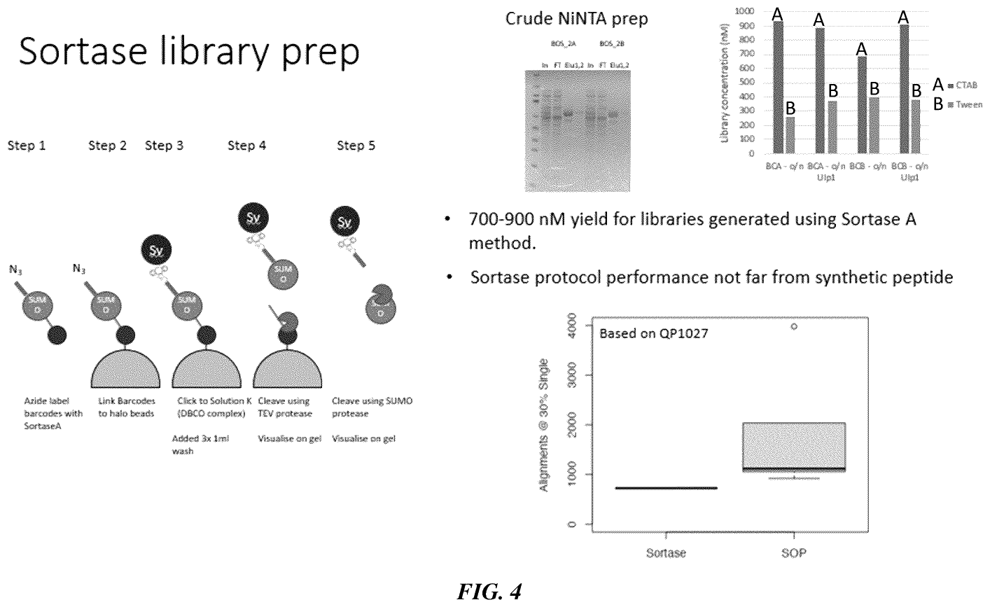

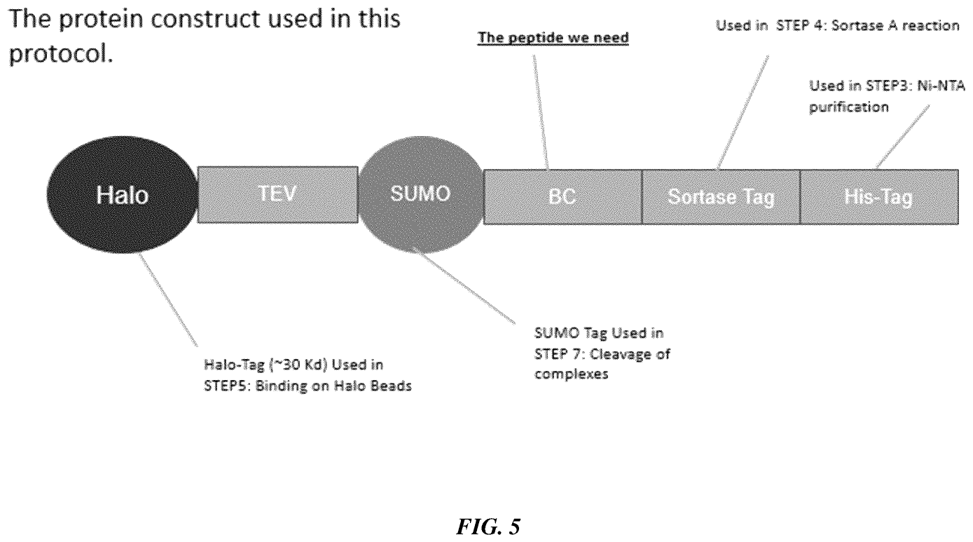

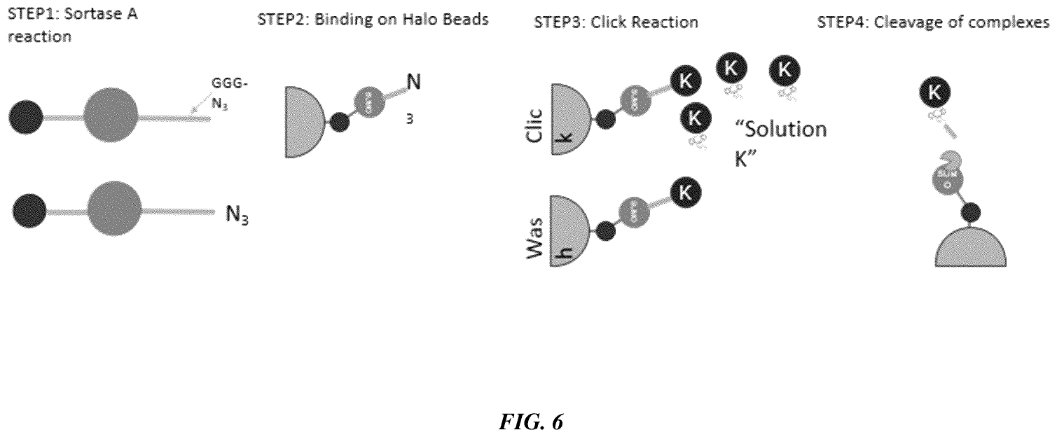

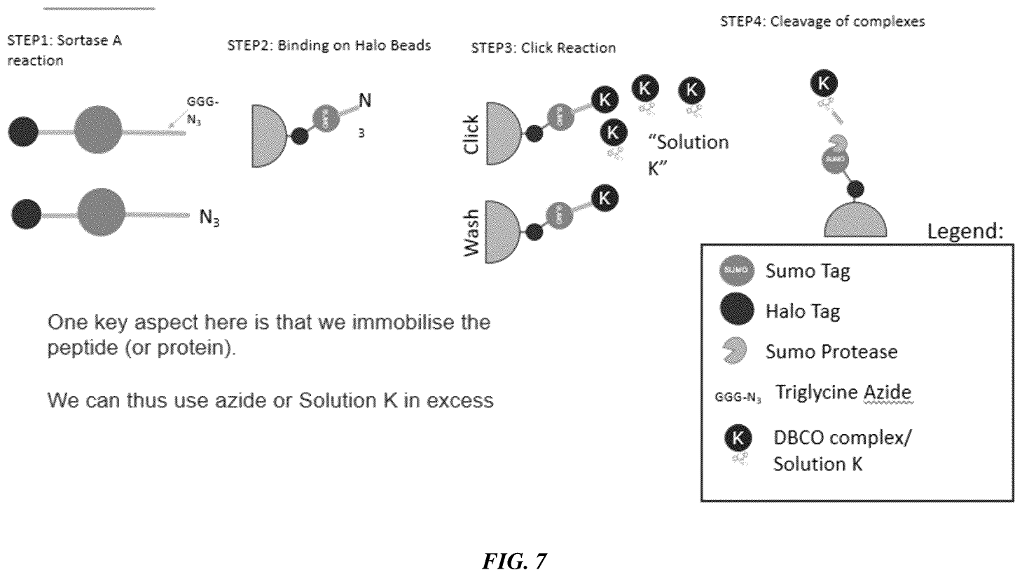

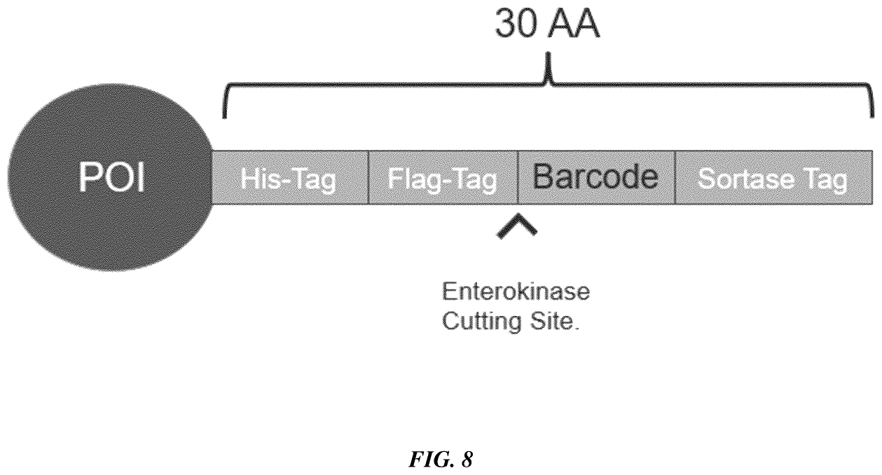

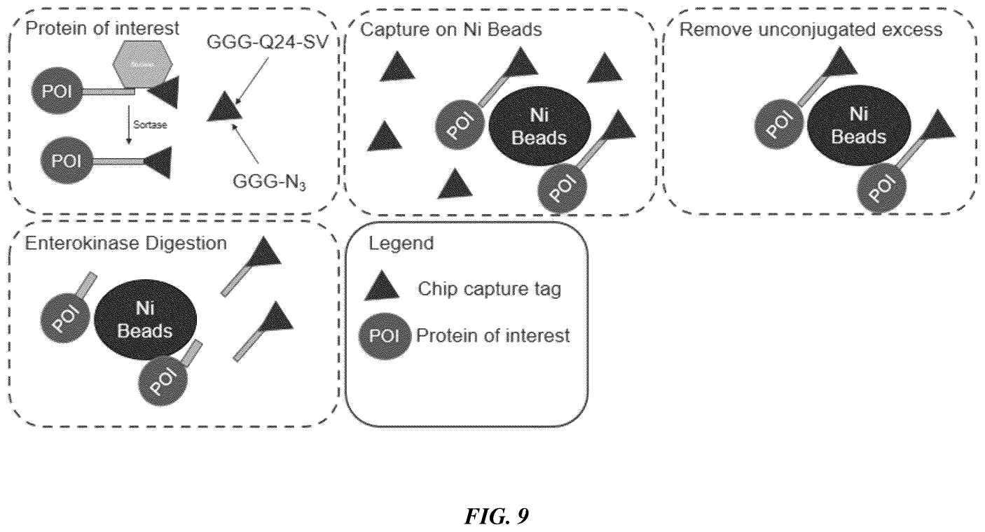

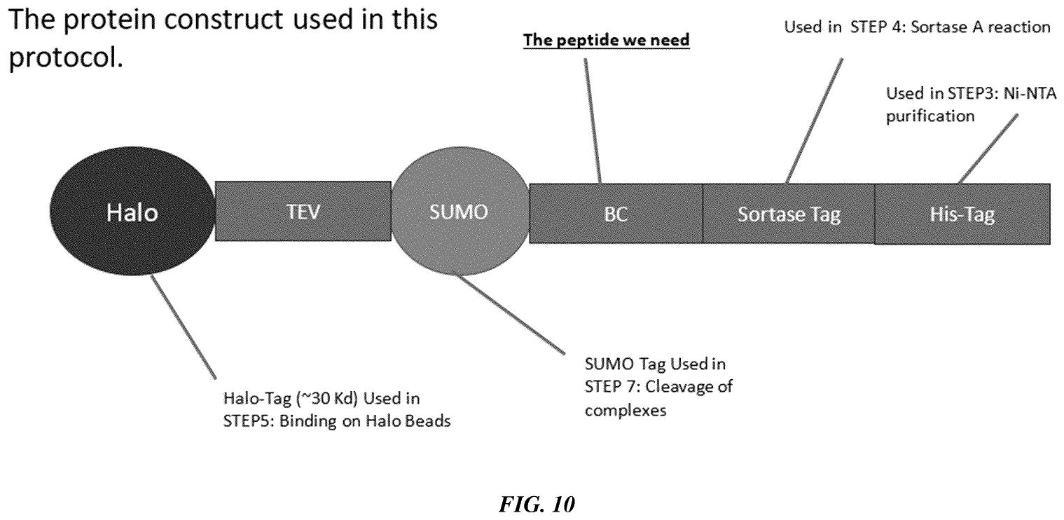

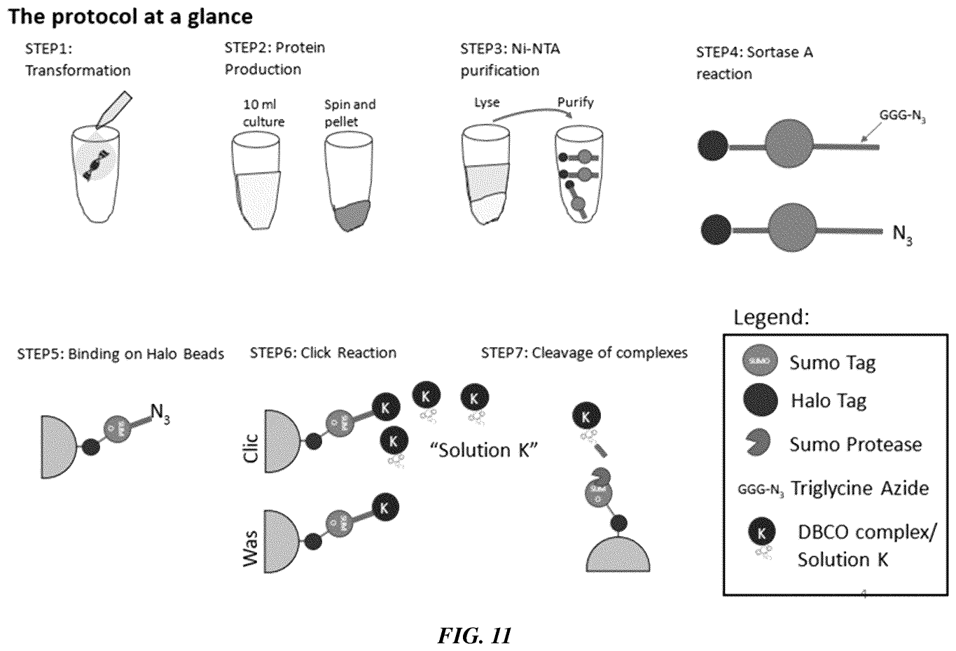

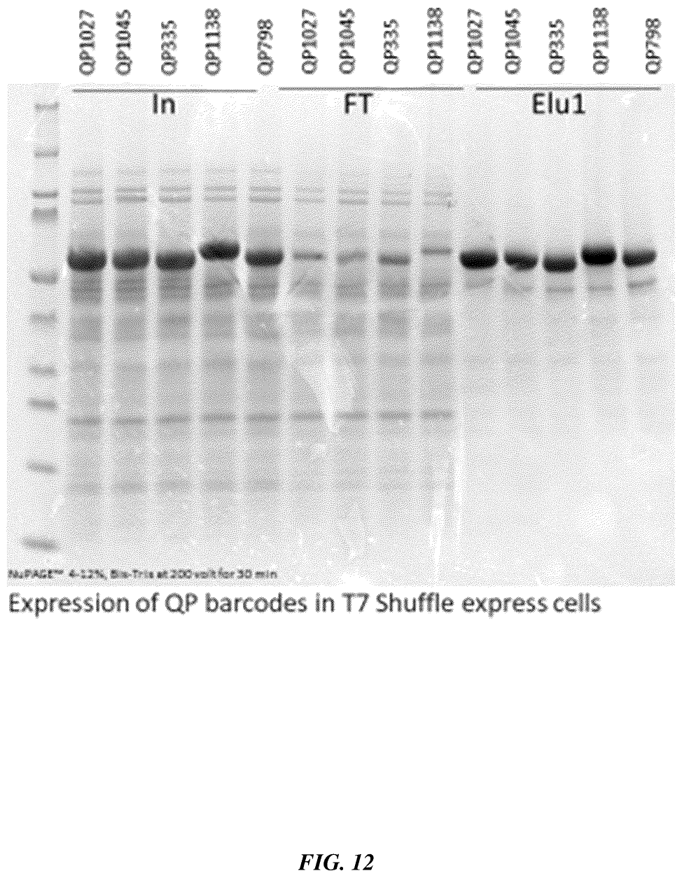

FIGS. 3A-3D show a stepwise process for enzymatic library preparation for peptide sequencing. Using Sortase A library preparation, protocol performance was higher than other library preparation methods (FIG. 4). Sortase A library preparation is quick and efficient, with high versatility, high yield, and good performance. In addition, library preparation using Sortase A can be carried out using minimal equipment. FIGS. 6, 7, 9, and 11 show further examples of library preparation using Sortase A.

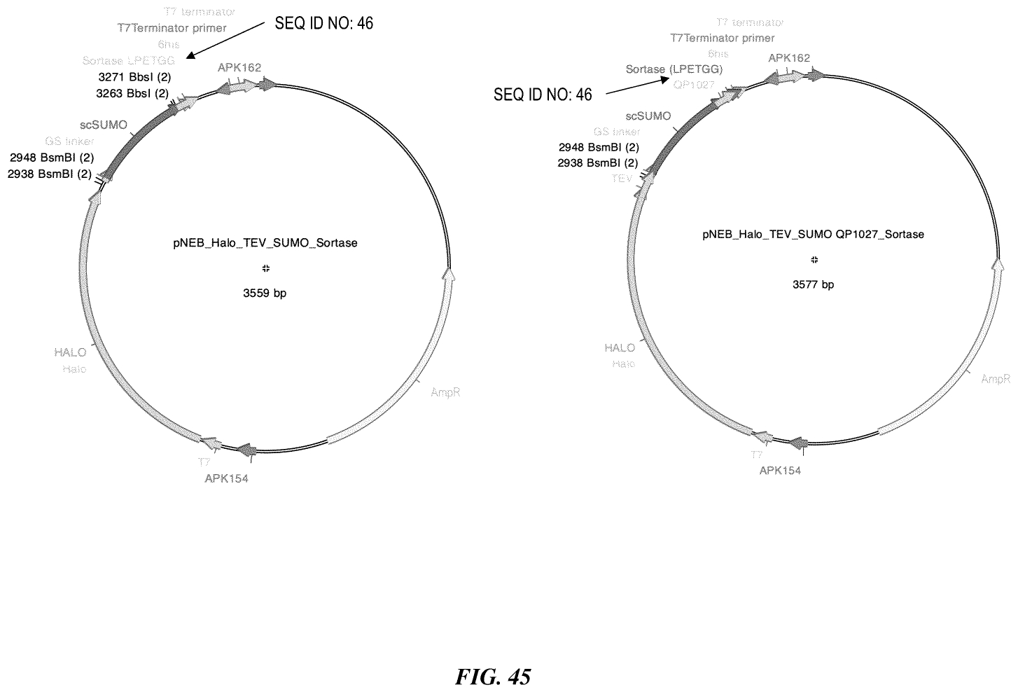

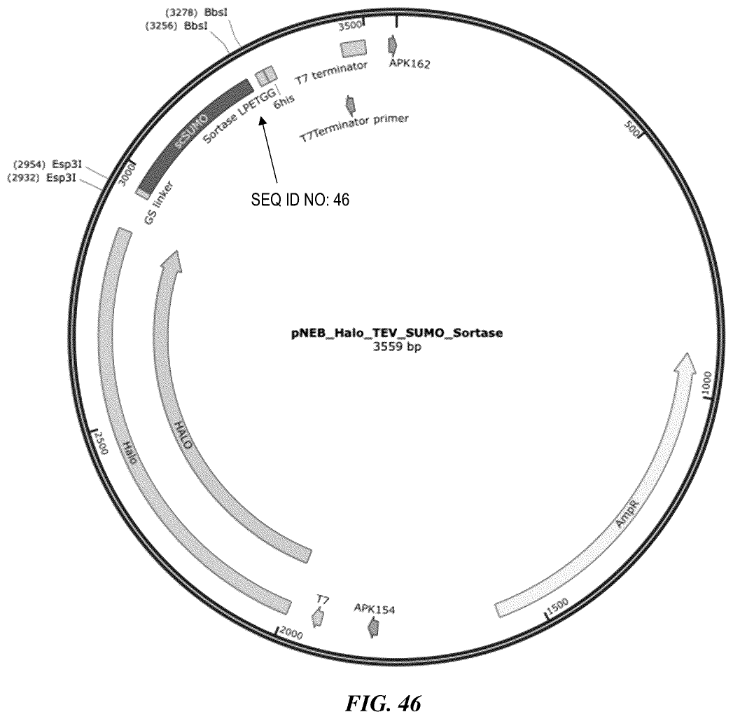

FIG. 45 shows the plasmids used in this protocol. FIG. 46 shows another plasmid used in this protocol. A protein of interest can be inserted between the Halo tag and GS linker using the Esp3I enzyme. The Barcode is inserted between the Sumo and Sortase sites using the BbsI enzyme. A non-limiting example of a nucleotide sequence of a plasmid used in this protocol (e.g., as shown in FIG. 46) is provided by SEQ ID NO: 47.

(SEQ ID NO: 47)
CGCTGCGCTCGGTCGTTCGGCTGCGGCGAGCGGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGCTAAGACACGACTTATCGCCACTGGCAGCAGCCACTG
GTAACAGGATTAGCAGAGCGAGGTATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTGATCCGGCAAACAAACCACCGCTGG
TAGCGGTGGTTTTTTTGTTTGCAAGCAGCAGATTACGCGCAGAAAAAAAGGATCTCAAGAAGATCCTTTGATCTTTTCTACGGGGT
CTGACGCTCAGTGGAACGAAAACTCACAGATCCGGGATTTTGGTCATGAGATTATCAAAAAGGATCTTCACCTAGATCCTTTTAAA
TTAAAAATGAAGTTTTAAATCAATCTAAAGTATATATGAGTAAACTTGGTCTGACAGTTACCAATGCTTAATCAGTGAGGCACCTA
TCTCAGCGATCTGTCTATTTCGTTCATCCATAGTTGCCTGACTCCCCGTCGTGTAGATAACTACGATACGGGAGGGCTTACCATCT
GGCCCCAGTGCTGCAATGATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAGTAAGTAGTTCGCCAGTTA
ATAGTTTGCGCAACGTTGTTGCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCTCCGATCGTTGTCAGAAGTAA
GTTGGCCGCAGTGTTATCACTCATGGTTATGGCAGCACTGCATAATTCTCTTACTGTCATGCCATCCGTAAGATGCTTTTCTGTGA
CTGGTGAGTACTCAACCAAGTCATTCTGAGAATAGTGTATGCGGCGACCGAGTTGCTCTTGCCCGGCGTCAATACGGGATAATACC
GCGCCACATAGCAGAACTTTAAAAGTGCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCAGCGTTTCTGGGTGAGCAAAAACAG
GAAGGCAAAATGCCGCAAAAAAGGGAATAAGGGCGACACGGAAATGTTGAATACTCATACTCTTCCTTTTTCAATATTATTGAAGC
ATTTATCAGGGTTATTGTCTCATGAGCGGATACATATTTGAATGTATTTAGAAAAATAAACAAATAGGGGTTCCGCGCACATTTCC
CCGAAAAGTGCTAGTGGTGCTAGCCCCGCGAAATTAATACGACTCACTATAGGGTCTAGAAGAAATAATTTTGTTTAACTTTAAGA
AGGAGATATACATATGGCGGAGATTGGCACAGGATTTCCATTTGATCCCCACTATGTTGAGGTTCTTGGTGAACGTATGCACTATG
TTGACGTTGGACCACGTGACGGAACCCCTGTTTTATTTTTGCATGGTAATCCGACTAGTAGCTACGTTTGGCGCAACATCATCCCA
CACGTCGCCCCTACTCACCGTTGTATCGCACCGGACTTGATCGGTATGGGGAAGTCCGACAAGCCAGACTTAGGCTATTTCTTTGA
CGACCATGTCCGTTTCATGGACGCGTTTATCGAGGCCCTTGGGTTAGAAGAGGTTGTACTTGTCATTCACGACTGGGGGTCCGCTC
TTGGCTTTCATTGGGCTAAACGTAACCCCGAGCGCGTCAAGGGGATCGCCTTTATGGAGTTTATCCGCCCCATCCCTACCTGGGAT
GAGTGGCCGGAATTTGCGCGTGAAACGTTTCAAGCCTTCCGCACAACTGATGTTGGCCGCAAATTAATTATCGATCAGAACGTGTT
CATCGAGGGGACATTGCCAATGGGAGTGGTTCGTCCGTTGACCGAAGTTGAAATGGACCATTATCGCGAACCGTTTCTGAACCCGG
TCGATCGTGAACCCTTATGGCGCTTTCCTAACGAATTGCCGATTGCTGGTGAACCTGCGAACATTGTCGCATTAGTAGAGGAATAC
ATGGACTGGTTACATCAATCCCCCGTTCCTAAATTGTTGTTTTGGGGAACGCCAGGCGTATTAATCCCGCCAGCGGAGGCCGCACG
CCTTGCGAAGTCACTTCCTAATTGCAAAGCAGTGGATATTGGCCCAGGCTTGAATCTTTTGCAAGAGGATAACCCTGATCTGATCG
GGTCGGAAATTGCGCGTTGGTTATCGACCCTTGAGATTTCGGGCACCAGCGAACCAACAACTGAGGACTTGTATTTCCAATCTGAC
AACGCAATCGCTAGAGACGGCTACGTCTCGGGTGGCGGATCTATGTCGGACTCAGAAGTCAATCAAGAAGCTAAGCCAGAGGTCAA
GCCAGAAGTCAAGCCTGAGACTCACATCAATTTAAAGGTGTCCGATGGATCTTCAGAGATCTTCTTCAAGATCAAAAAGACCACTC
CTTTAAGAAGGCTGATGGAAGCGTTCGCTAAAAGACAGGGTAAGGAAATGGACTCCTTAAGATTCTTGTACGACGGTATTAGAATT
CAAGCTGATCAGACCCCTGAAGATTTGGACATGGAGGATAACGATATTATTGAGGCTCACAGAGAACAGATTGGTGGTAGGTCTTC
GAGAAGACCTTTGCCTGAAACCGGCGGACATCACCACCATCATCACTGACTCGAGTAAGGTTAACCTGCAGGAGGCCTTTAATTAA
GGTGGTGCGGCCGCGCTAGCGGTCCCGGGGGATCGATCCGGCTGCTAACAAAGCCCGAAAGGAAGCTGAGTTGGCTGCTGCCACCG
CTGAGCAATAACTAGCATAACCCCTTGGGGCCTCTAAACGGGTCTTGAGGGGTTTTTTGCTGAAAGGAGGAACTATATCCGGAAGC
TTGGCACTGGCCGACCGGGGTCGAGCACTGACT

FIG. 47 shows a non-limiting example of a protein construct, which contains a protein of interest and a barcode, prepared from a plasmid used in this protocol (e.g., as shown in FIG. 46).

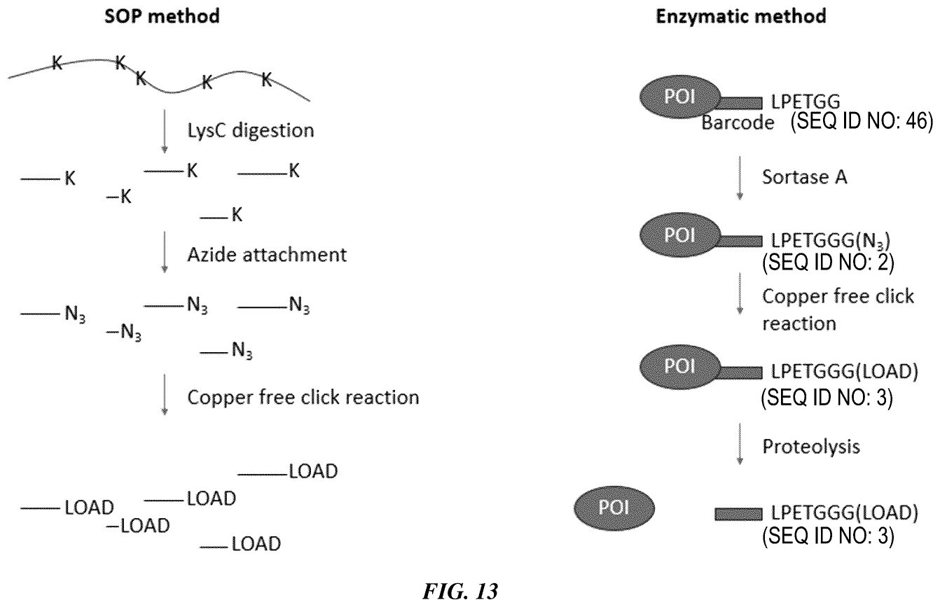

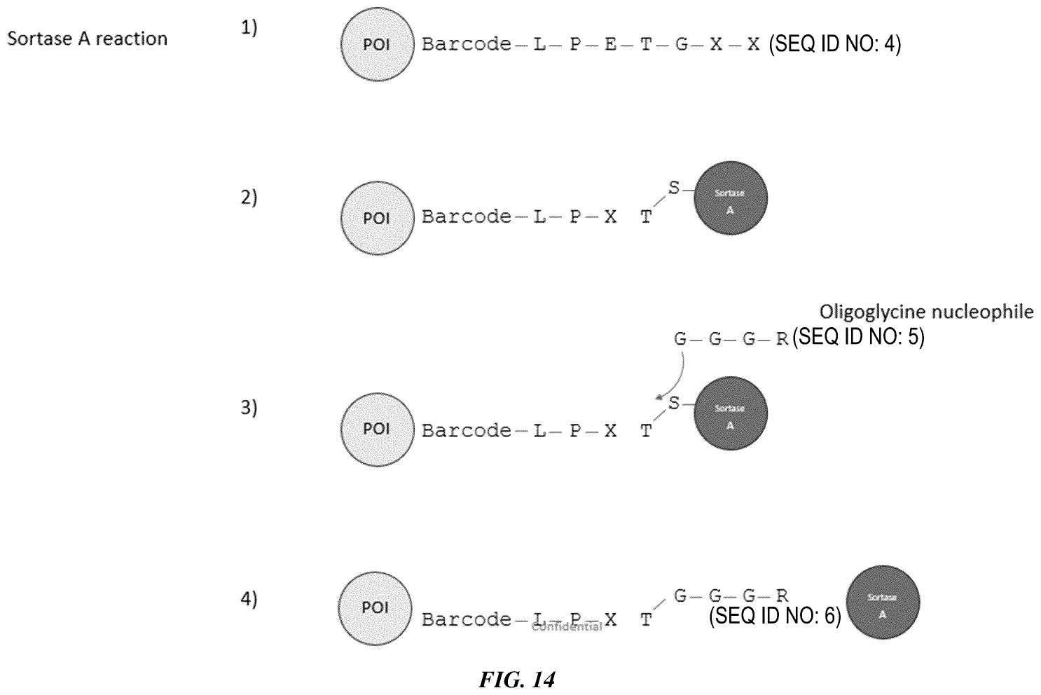

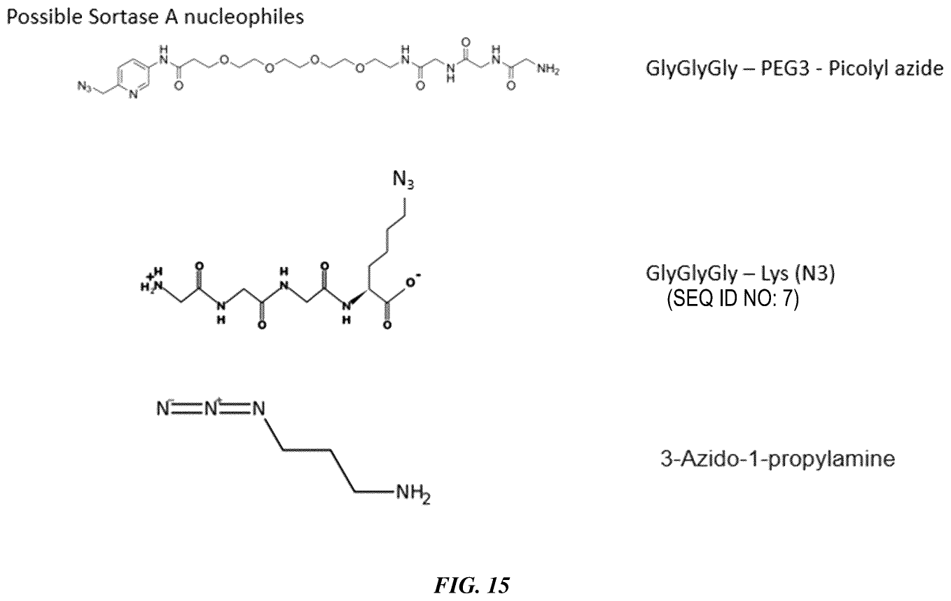

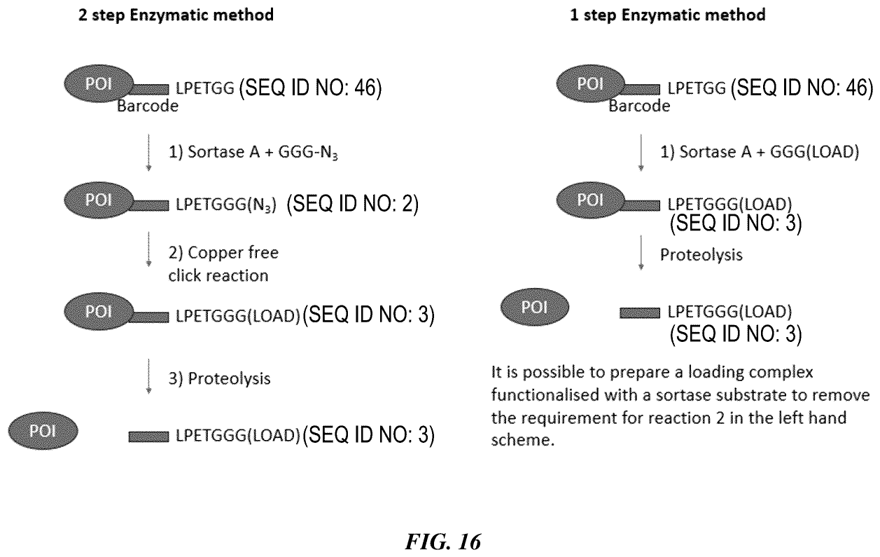

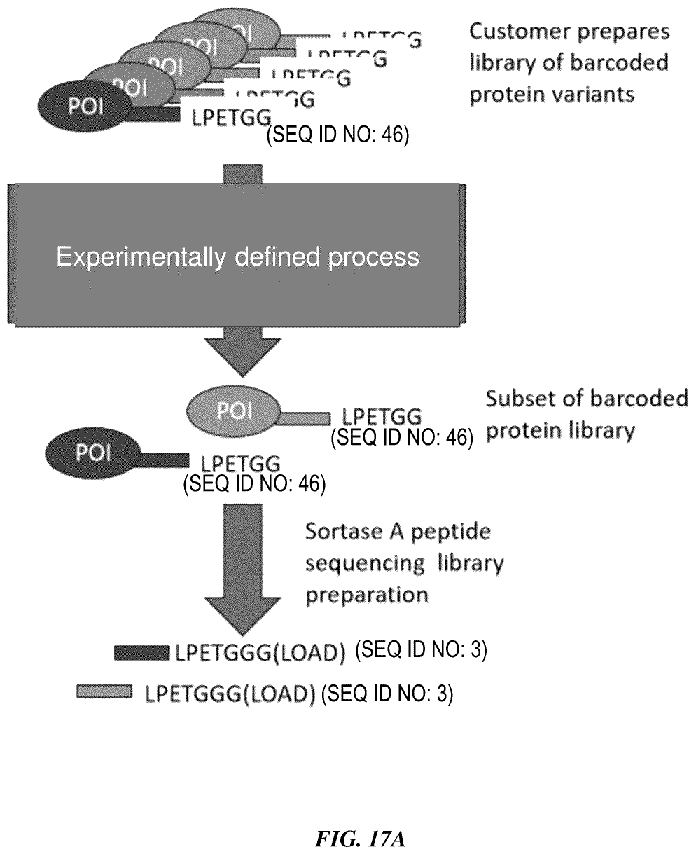

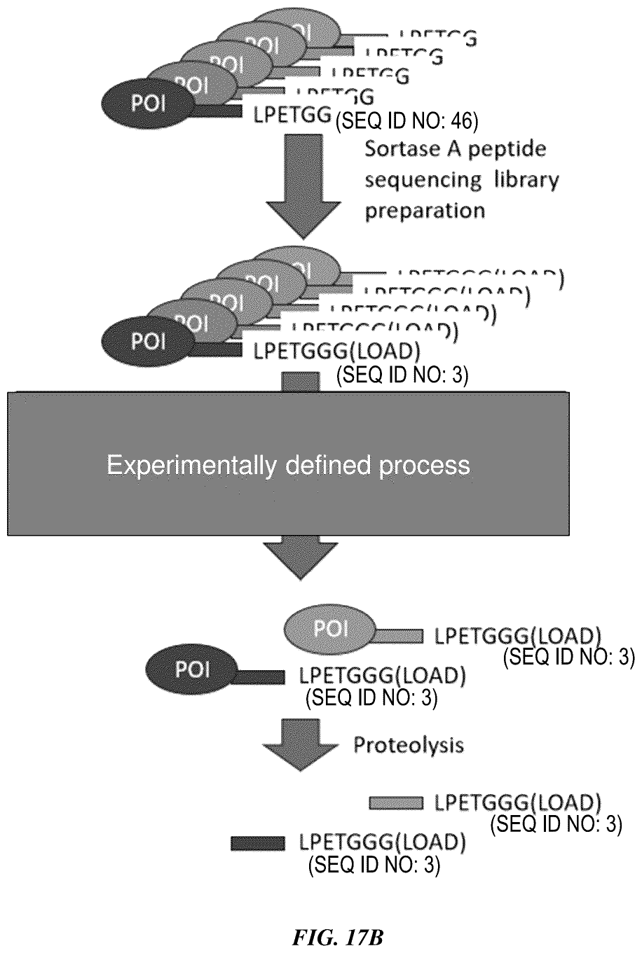

FIGS. 13-17B show the workflow for enzymatic library preparation for peptide sequencing.

### Example 3. Error-Resistant Barcode Design and Application

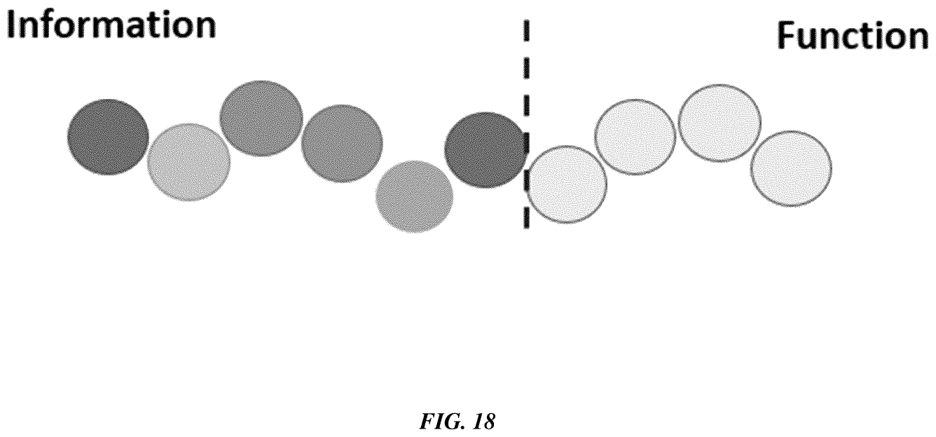

This example describes a method to design amino acid sequences (“peptide barcodes”) to be used in single-molecule protein sequencing applications. The example is also related to peptide barcode compositions, that is, amino acid sequences that can be expressed or synthesized on their own or appended to a protein. Peptide barcodes contain an “information” region and, when needed, a “functional region (FIG. 18).

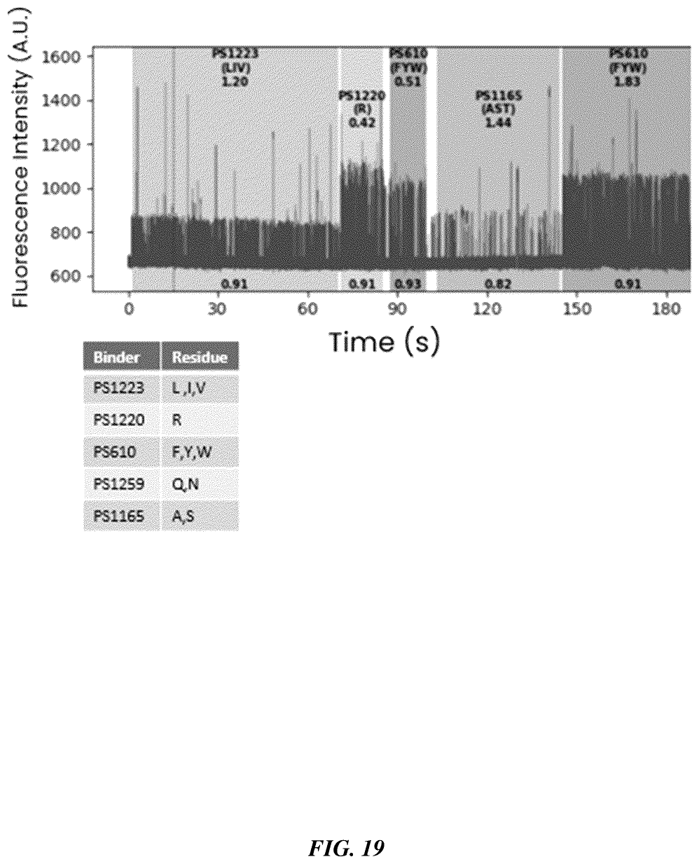

An amino-acid sequence, when sequenced gives rise to a complex signal (FIG. 19). In this signal, Regions Of Interest (ROIs) are identified, as shown by the colored segments in FIG. 19. Each ROI represents an amino acid of the sequence, but not all amino acids give rise to ROIs (some amino acids are not recognized depending on the reagents present in a sequencing reaction). ROIs stem from the on-off binding of a recognizer to the N-terminal amino acid. Transitions between ROIs depend on enzymatic cutting of the N-terminal amino acid, resulting in the exposure of a different N-terminal amino acid. Recognizers can have multiple substrates, and a single recognizer can bind several N-terminal amino-acids (FIG. 20). Table 2 shows example recognizers.

TABLE 2

| Binder | Amino Acid Residue |
|---|---|
| PS1223 | Leucine (L), Isoleucine (I), Valine (V) |
| PS1220 | Arginine (R) |
| PS610 | Phenylalanine (F), Tyrosine (Y), Tryptophan (W) |
| PS1259 | Glutamine (Q), Asparagine (N) |
| PS1165 | Alanine (A), Serine (S) |

A signal is interpreted via its ROIs. In one example sequencing reaction, each ROI is generated by one of five recognizers, labelled with a specific dye, that emits light with specific properties. This is the first trait characterizing ROIs. Different recognizers can be distinguished by different colors (although the property of the dye that distinguishes them is lifetime, also called ‘bin ratio’ and not the emission spectrum). Within an ROI, consistent pulsing by one recognizer can be observed (although mixed ROIs if recognizers would have overlapping specificities are conceivable).

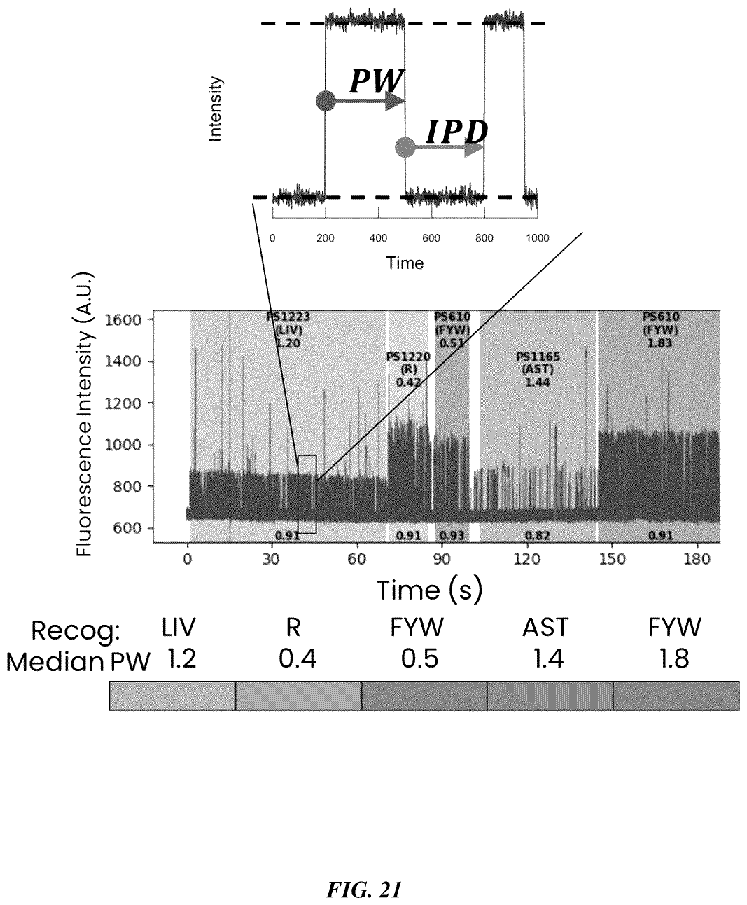

Within an ROI, the signal is statistically consistent. Each pulse in the ROI will have a Pulse Width (PW) and an Inter Pulse Distance (IPD) (FIG. 21, top panel). The median pulse width and the median IPD are distinctive of the ROIs and sensitive to the nature of the N-terminal amino-acid and of the context (penultimate amino-acid and beyond). The signal from one aperture is often idealized as a ‘kinetic signature’ (FIG. 21, bottom panel) that summarizes the properties of the ROIs. Two properties that are typically used are ‘color’ distinguishing the different recognizers and median PW (as in the example here). Other properties might also be used, e.g., median IPD.

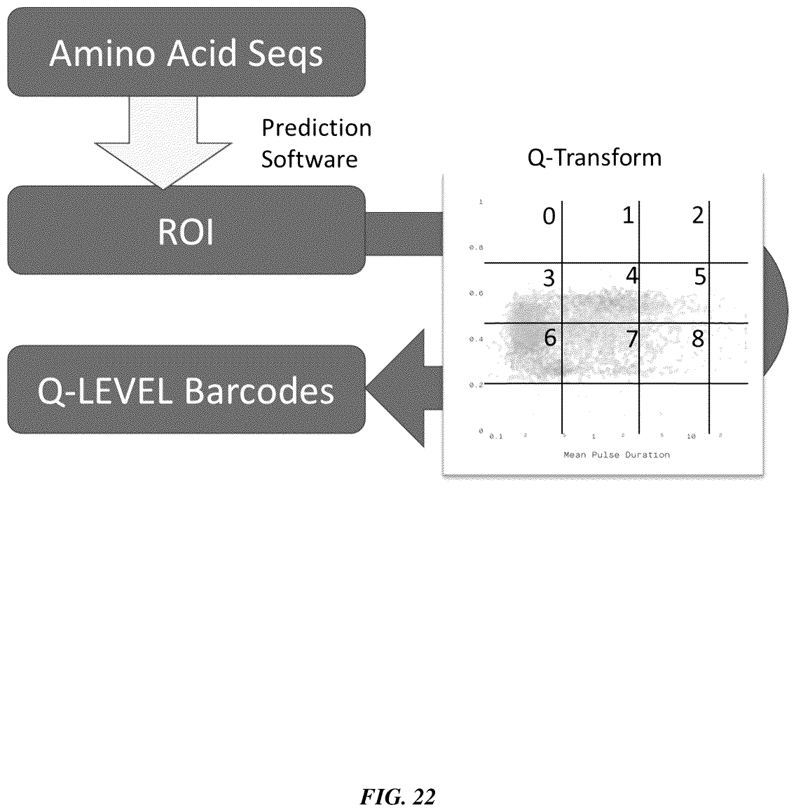

Barcodes are physically amino acid sequences. The pulsing properties of each residue along the amino acid barcode (ROIs) are capable of being modeled on chip. ROIs are described by multiple parameters including binratio (average or distribution), pulse width (average or distribution), interpulse distance (average or distribution). The parameter space for the ROIs can be discretized in blocks (e.g., discretization based on mean binratio and mean pulse width). The number of blocks will be denoted by Q, each of the Q blocks will be associated to an integer {0, . . . , Q−1}. The Q-transform is the process by which ROIs are mapped on an amino acid barcode to a block and thus to an integer. An amino acid sequence then becomes (through it ROIs) a series of integers A1 . . . AN with Ai in {0, . . . , Q−1} associating amino acid barcodes to abstract Q-Level barcodes. See FIG. 22.

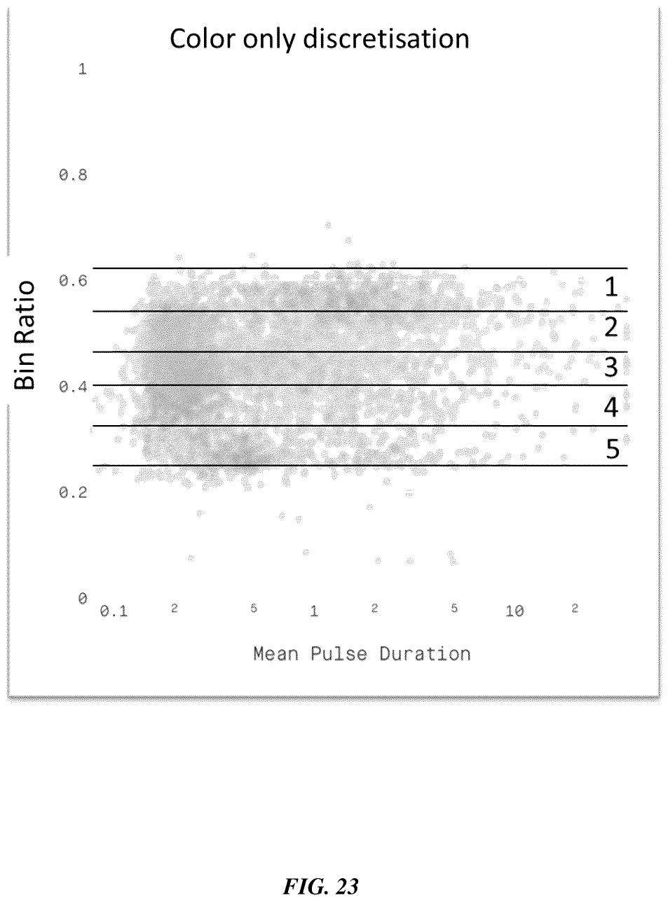

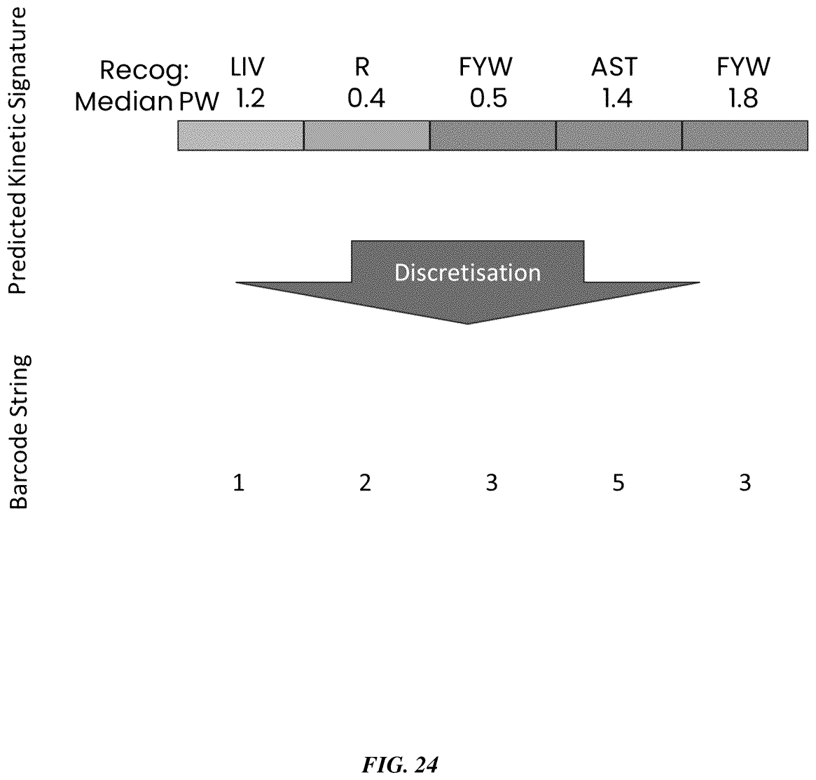

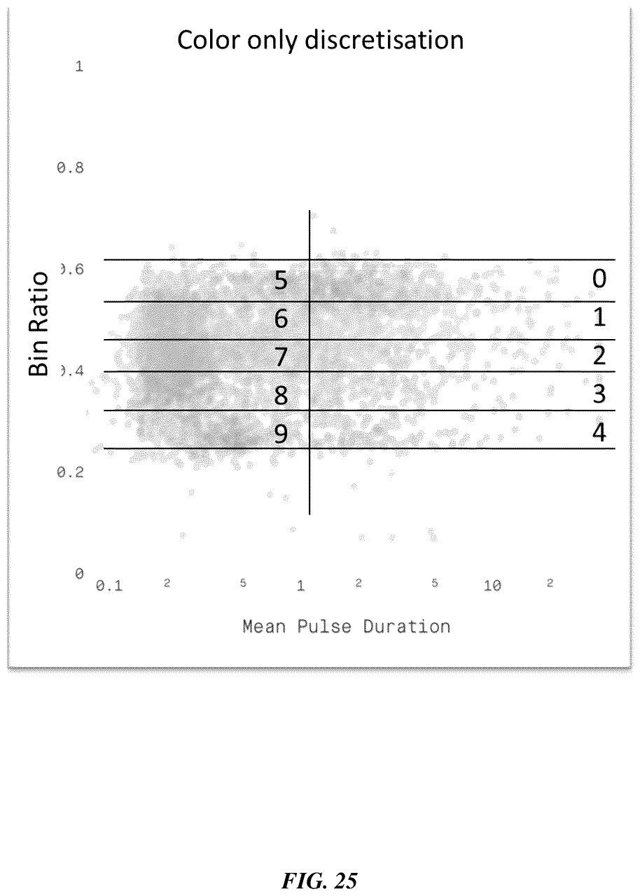

As detailed previously, an empirical ROI can be identified by several parameters, notably, but not exclusively, PW and binratio. These parameters can define the space of possible ROIs. To obtain digital barcodes from kinetic signatures, a discretization is imposed on the space of ROIs, which can be done in many ways. In FIG. 23, only the color (binratio) of the ROI is considered. Although many different ROIs may give the same color, they may be considered equivalent for the purposes of certain analyses. For example, barcodes with 5 ROIs in this ‘color only’ discretization give a total diversity of 625 possible barcodes (5*5*5*5). In a different example, one may differentiate between those ROIs that have median pulse duration above 1 seconds and those that have MPD less than 1 second (e.g., 10 bins and with 4 ROIs would have a total diversity of 10,000). See FIG. 25.

### Discretization

This example describes how the two different discretization strategies will make barcode decoding robust. In both cases, for example, the median pulse width of each ROI need not be measured with accuracy. In the first case, median pulse width is disregarded. In the second case, ROI kinetics are evaluated only to determine if it is above or below 1 second. This allows for a larger number of barcodes that may be generated with this second scheme (10k vs 625 with 4 ROI).

Having turned barcodes into simple discrete sequences, they can be analyzed using concepts from digital signal processing. One example is error correction. One can generate 625 barcodes from 4 ROIs with color-only discretization, which puts together all the possible sequences of four symbols with alphabet {1,2,3,4,5}: 1111, 1112, 1113, 1114[ . . . ]. It may be desired to restrict the number of sequences used in certain experiments. For example, if both 12523 and 12423 are in a set of barcodes, one single deletion of the central ROI would make one unable to assign the remaining read to either reference.

### Barcode Design

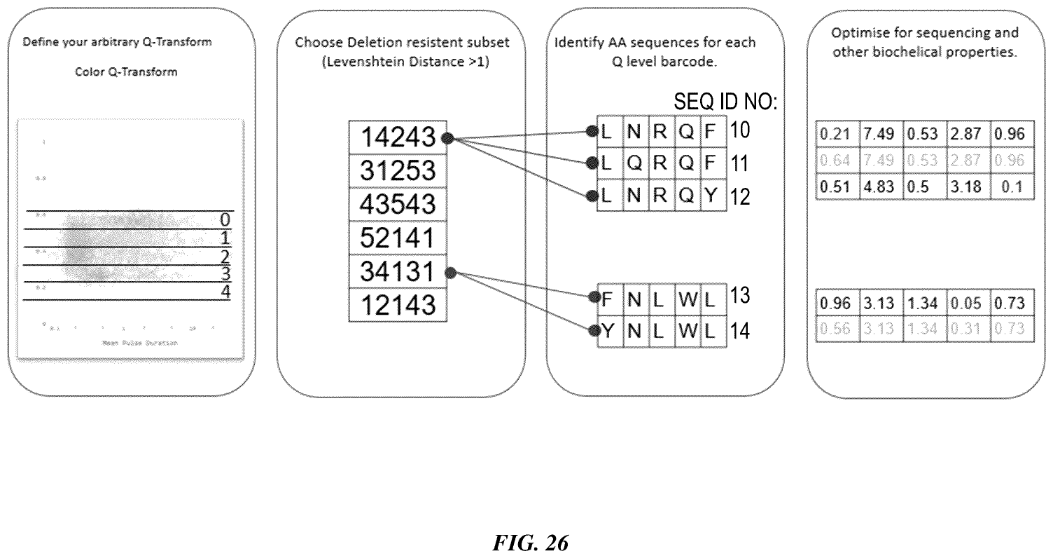

Barcode Design may start from the choice of a discretization of ROI space. In this example, it was decided to discretize only on bin ratio. Each block in the discretization is then associated with an integer number (or a symbol in a finite alphabet). Once the discretization scheme is set, the full vocabulary of q-level barcodes that will be available have been defined. This vocabulary can then be partitioned in subsets such that each q-level barcode in the subset has Levenshtein distance>n to each other q-level barcode in the subset. These sets of q-level barcodes are robust to errors. Each q-level barcode is associated to multiple amino-acid sequences that fall in the same series of blocks. This mapping can be identified using a model of peptide sequencing as described herein. From the multiplicity of sequences associated with a q-level barcode, one is selected based on its pulsing behavior and on a variety of in silico predicted biochemical features (e.g., hydrophobicity, isoelectric point). This provides a unique set of barcodes, error resistant and amenable to peptide sequencing as described herein. See FIG. 26.

In certain implementations, it may be useful to introduce extra amino acid(s) in the barcode sequence that are not recognized (do not give pulsing) but optimize kinetics upstream (e.g., to maximize information at the N-terminus (initial ROIs in the barcode set)). It may also be useful to avoid repeating strings (e.g., 121212) and/or two barcodes sharing a common substring (e.g., 31234 and 51233).

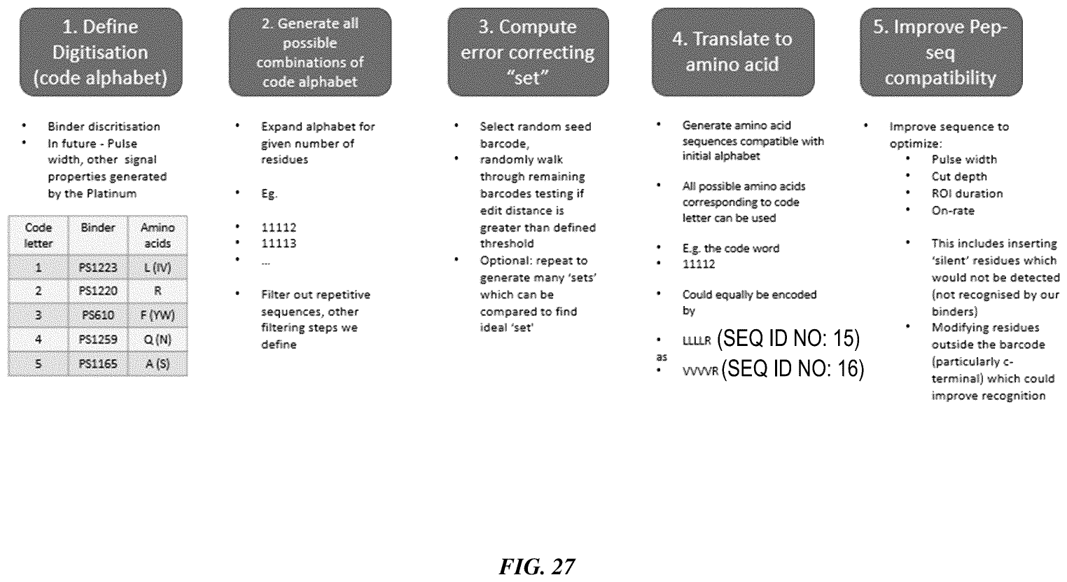

FIG. 27 shows an example barcode generation algorithm.

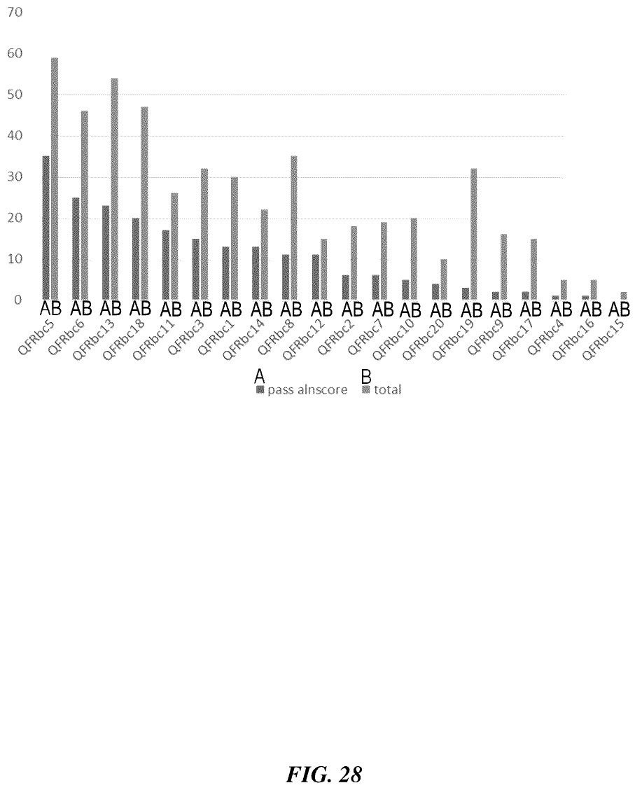

FIG. 28 shows a first design iteration. Made for 3 binder system. Sequences designed to maximize state transitions (not binder-order design). High FDR (0.44). Relatively few traces (max 35 pass aln score). POC for enzymatic library prep.

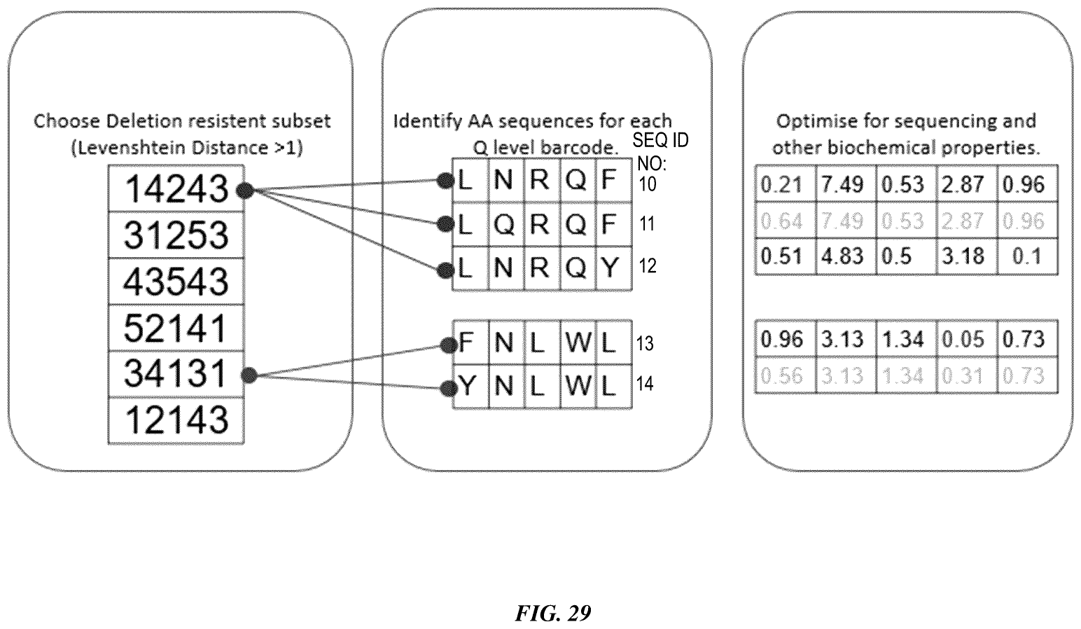

FIG. 29 shows a second design iteration. Made for a 5 binder system, first attempt at a binder-order-sequencing barcode system, generated 1 set of 100 barcodes in 2 subsets of 50.

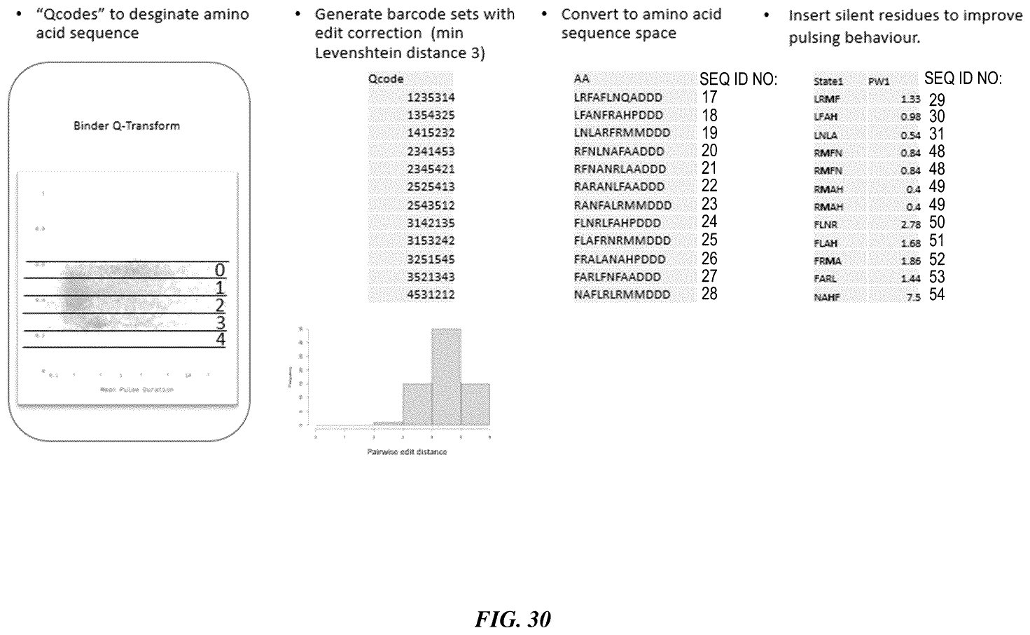

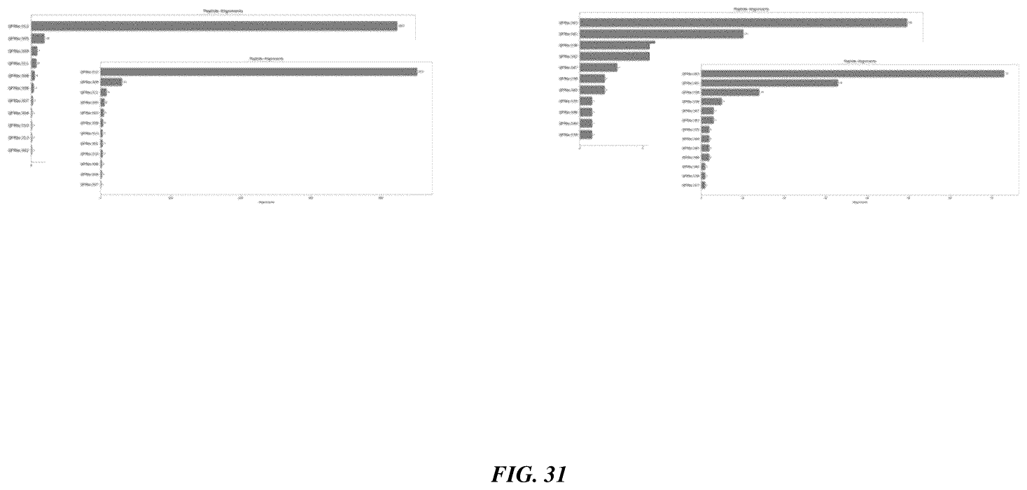

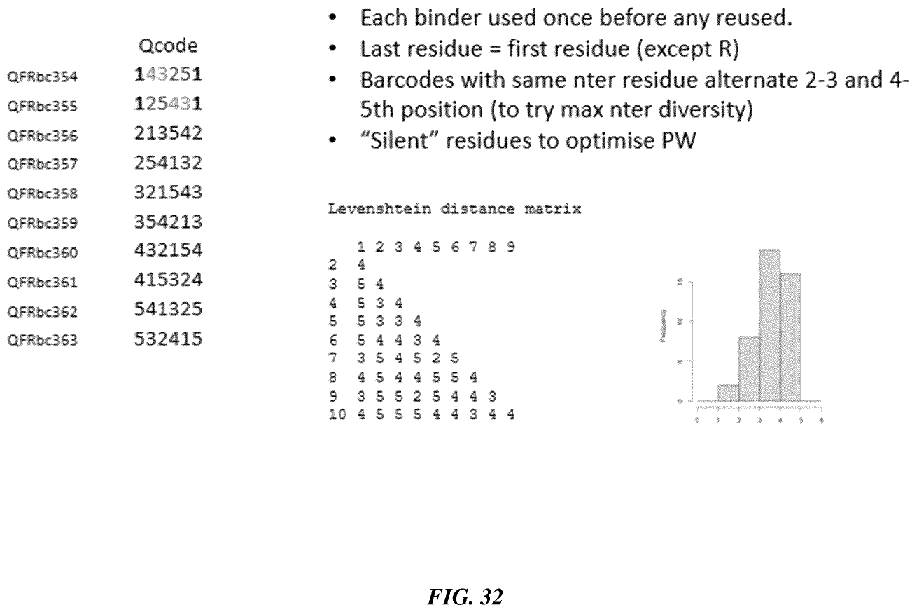

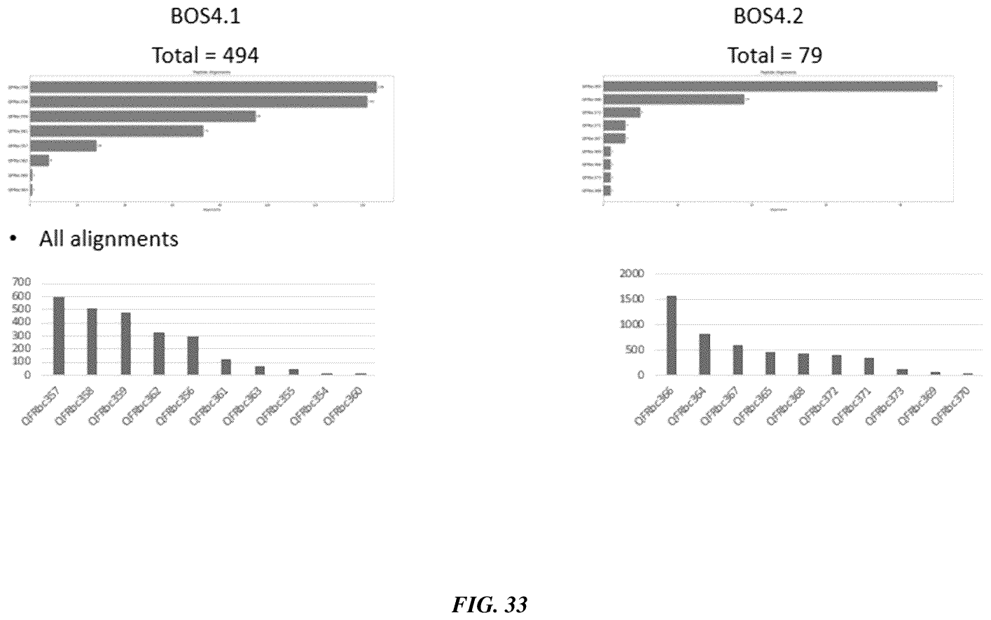

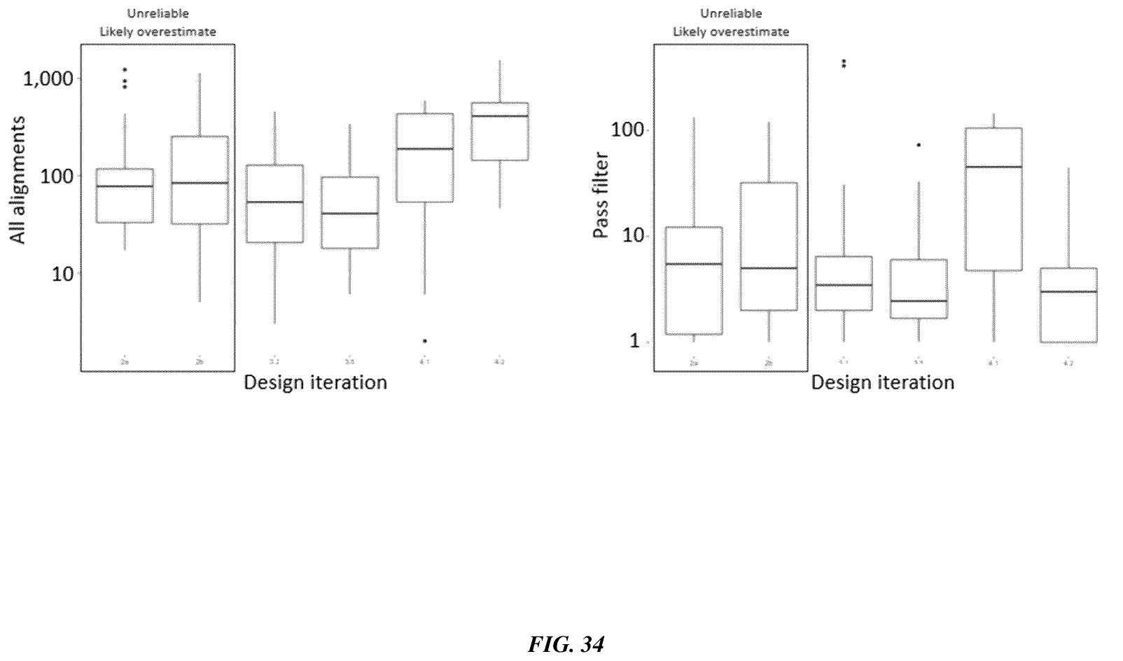

FIGS. 30-33 show additional iterations of barcode design.

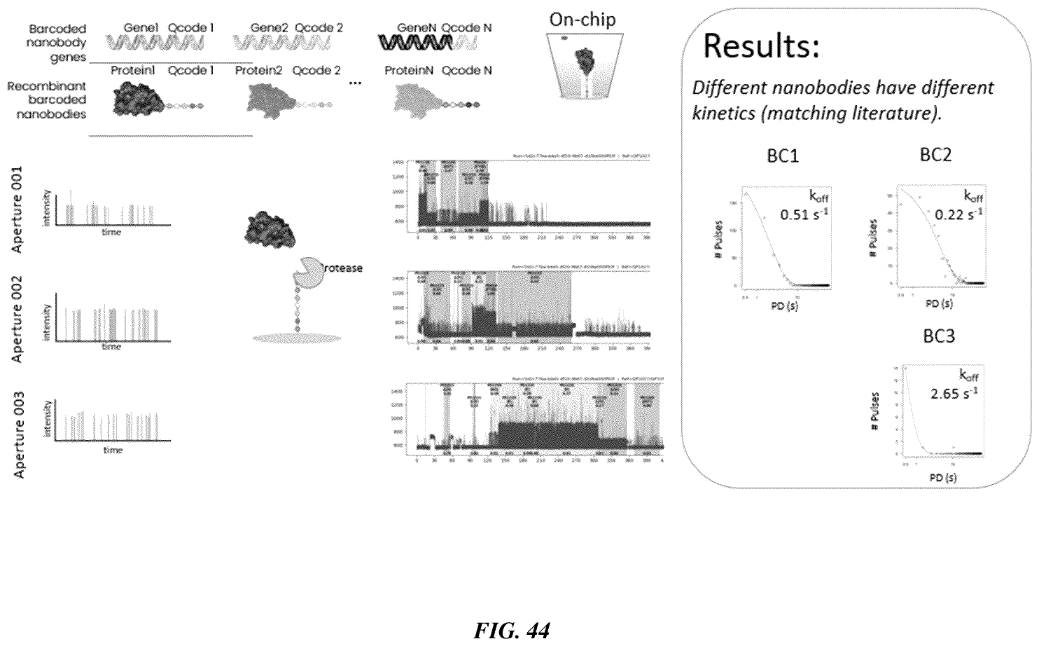

FIG. 44 shows a quantitative assessment of the design iterations described herein.

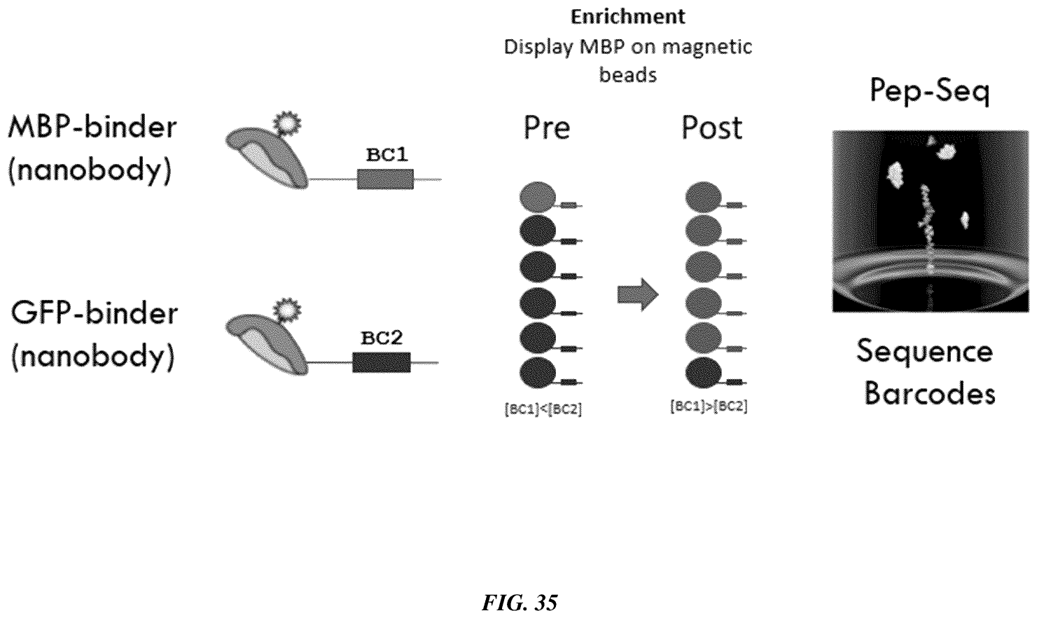

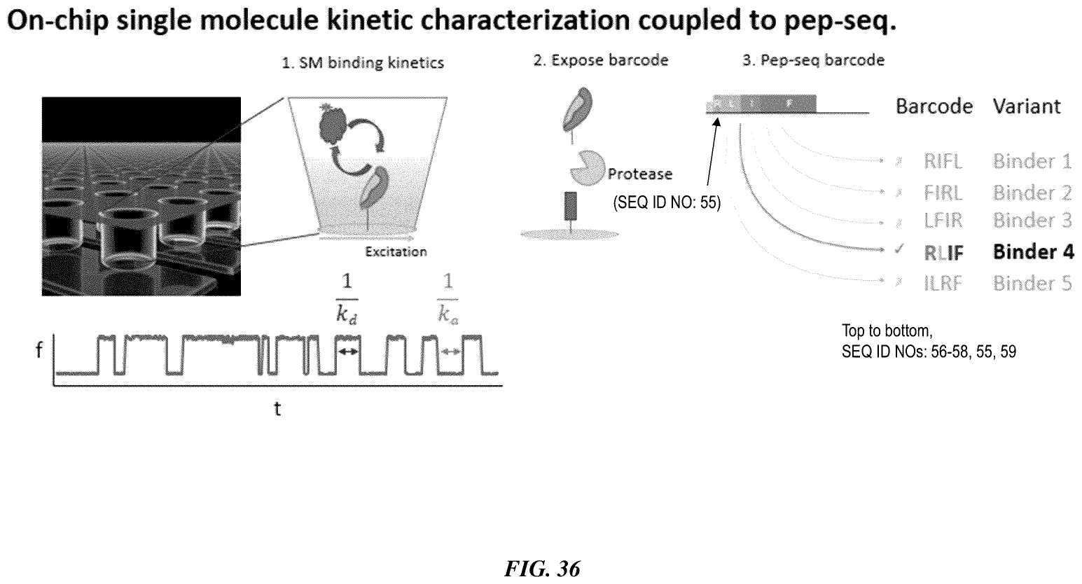

FIGS. 35-36 show applications of peptide barcodes.

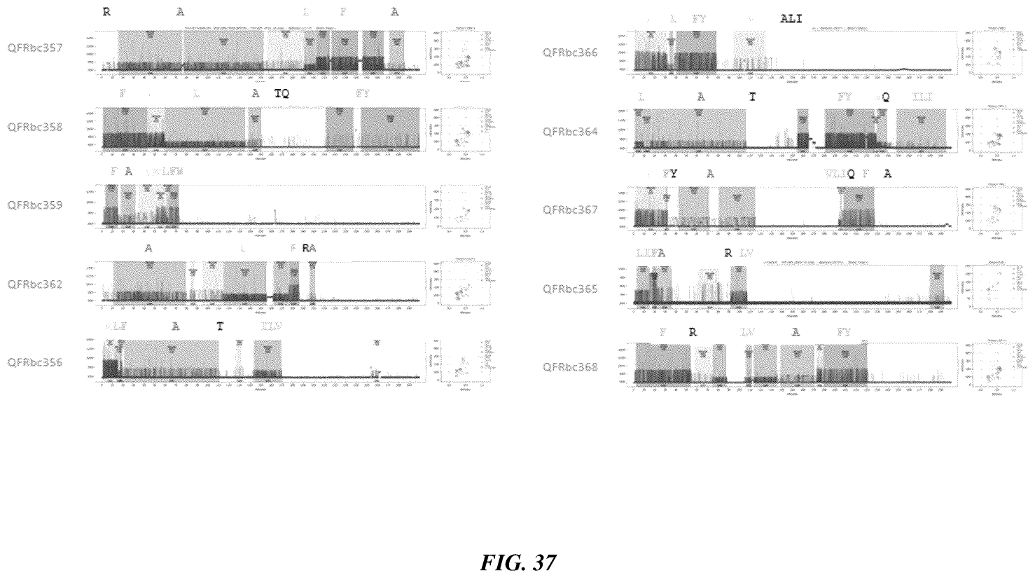

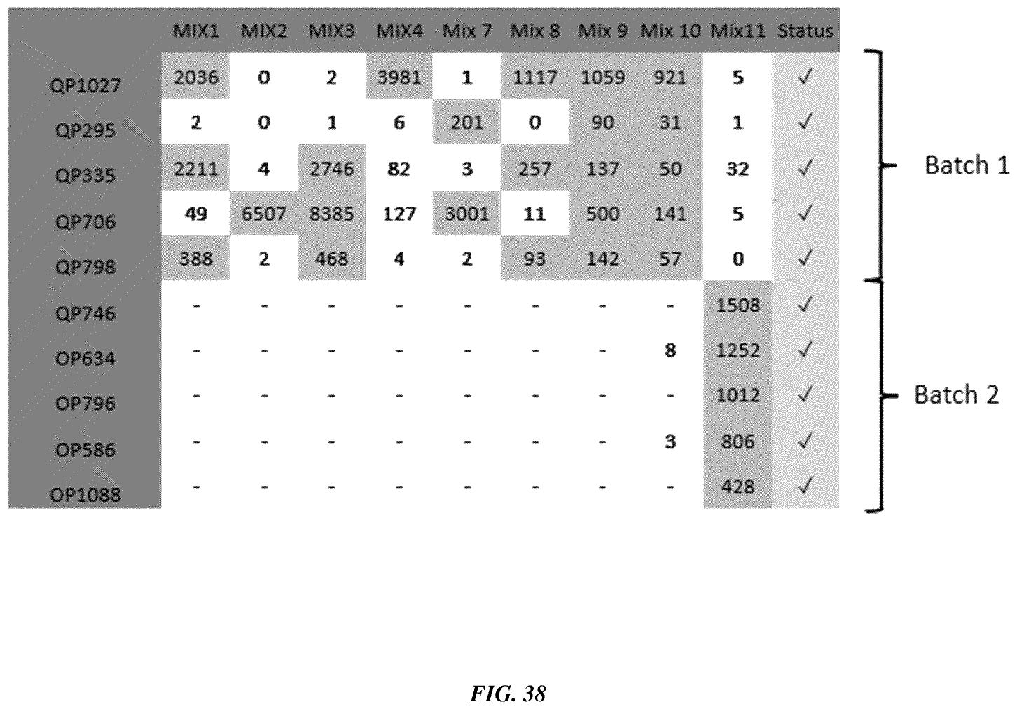

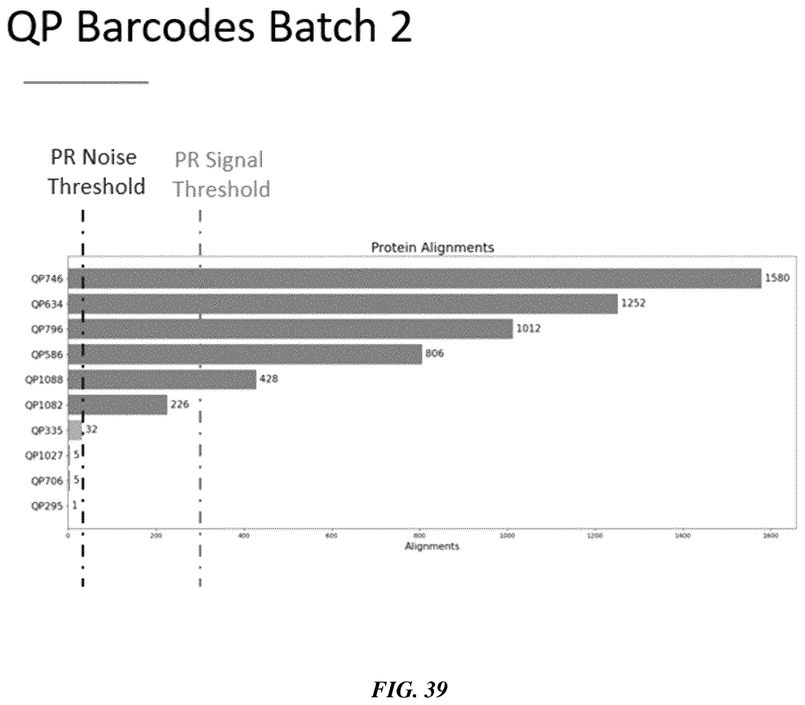

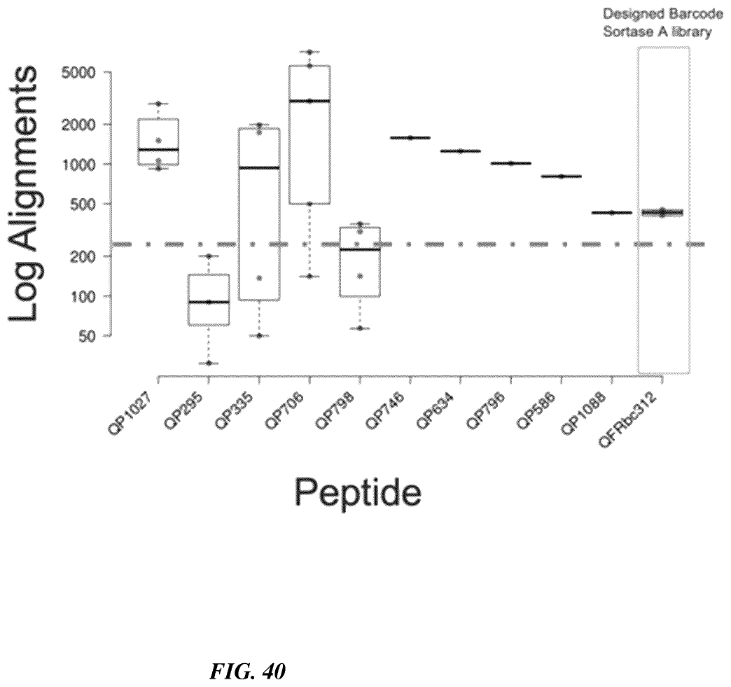

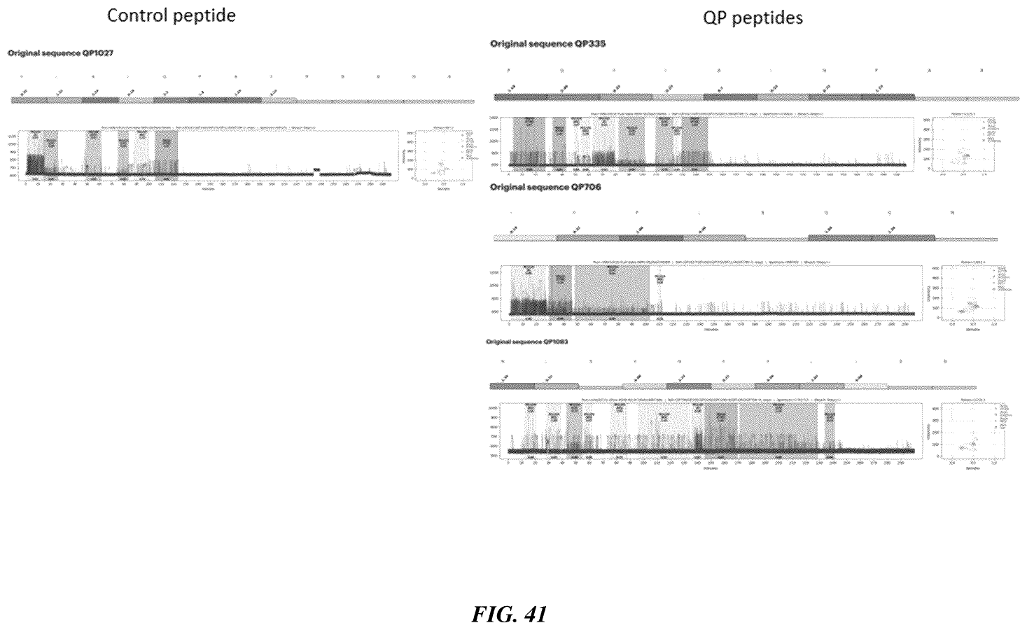

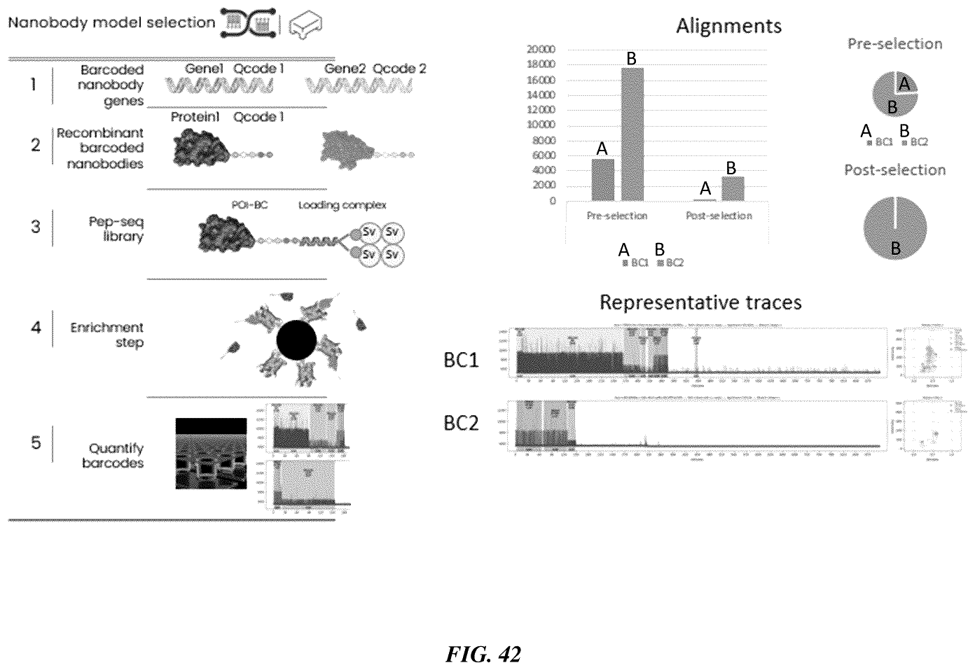

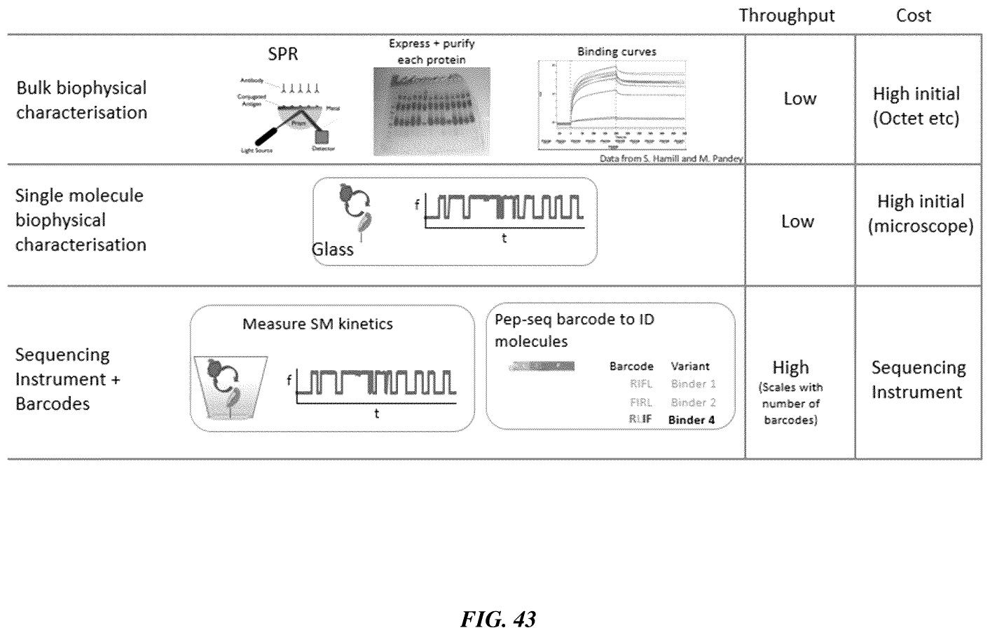

FIG. 37 shows top traces for the five highest performing barcodes.

### Example 4. Single Molecule Peptide Barcode Sequencing

Biological samples, including fresh and frozen formalin-fixed paraffin-embedded tissue sections, contain thousands of different target analytes, including proteins and mRNA transcripts. This example relates to the use of peptide barcodes of the present disclosure to determine the identity of one or more target analytes in a biological sample.

Prior to determining the identity of one or more target analytes within a cell, an image of a tissue sample is captured to allow mapping of peptide barcode information onto the image of the tissue sample. To determine the identity of one or more target analytes in a biological sample, an affinity reagent, such as an oligonucleotide or a protein-based molecule, is conjugated to a peptide barcode, either directly or indirectly via a linker. The affinity reagent is conjugated to a peptide barcode by expression of a polynucleotide encoding the affinity reagent and the peptide barcode, either within a cell within a tissue sample or prior to introduction of the affinity reagent conjugated to a peptide barcode into a cell within a tissue sample. Once inside a cell, an affinity reagent binds to a target analyte within the cell. This step can be multiplexed to target several target analytes within several cells by delivering or expressing multiple affinity reagents each conjugated to a peptide barcode to or in cells within a tissue sample.

Following binding of an affinity reagent to a target analyte, unbound affinity reagents are removed from the tissue sample. Removal of unbound affinity reagents is accomplished by contacting the tissue sample with a wash buffer. Once only bound affinity reagents remain, peptide barcodes are released from their respective affinity reagents and collected for sequencing following the sequencing methods described in the present disclosure.

Release of peptide barcodes from their respective affinity reagents is accomplished by one of several techniques. Release is accomplished by contacting an affinity reagent conjugated to a peptide barcode with an endopeptidase or a light that cleaves a linker (e.g., a photocleavable linker) that is connecting the affinity reagent and peptide barcode. Use of a photocleavable linker to indirectly attached a peptide barcode to an affinity reagent is advantageous for retaining spatial information. A laser can be used to target a particular user-specified region within a tissue sample so that only peptide barcodes within the user-specified region are released from their respective affinity reagents. Because each peptide barcode is specific to its respective affinity reagent, the presence of sequencing reads derived from a specific peptide barcode indicates that the target analyte to which the affinity reagent binds is present within the tissue sample. Peptide barcodes collected from a particular user-specified region are further mapped to the user-specified region, thus resulting in data indicating the spatial location of a particular target analyte within a tissue section.

### Example 5. Screening Drug Delivery Methods Using Peptide Barcodes

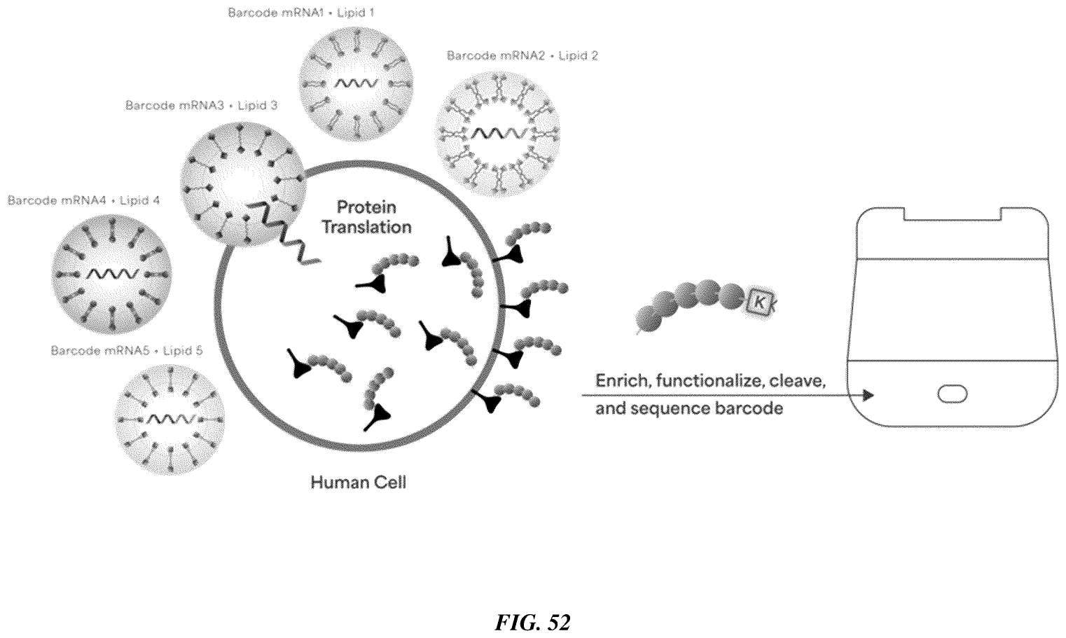

The peptide barcodes of the present disclosure can be applied to many areas of research. This example describes the use of the peptide barcodes of the present disclosure to screen methods of drug delivery (FIG. 52).

Lipid nanoparticles (LNPs) are particularly useful for the delivery of therapeutics to target cells. Unlike other therapeutic delivery methods, LNPs can be engineered to minimize immune response and target specific cells within a biological system. Engineering LNPs, however, is laborious and expensive and does not always result in effective LNPs for an intended purpose. Challenges associated with LNP efficacy can be overcome using the peptide barcodes of the present disclosure.

LNPs are designed to contain an mRNA molecule encoding unique barcodes. Once an LNP delivers its payload to a target cell, the mRNA molecule encoding a unique barcode is translated by the cell's endogenous translation system to produce peptide barcodes. The mRNA molecule can also encode for a receptor to shuttle the peptide barcode to the cell surface. Once in the cell or on the cell surface, the peptide barcode can be cleaved, collected, and sequenced. The presence of sequencing reads from sequencing a particular peptide barcode is indicative of the efficacy of a particular LNP. The sequencing reads are only present if the LNP effectively delivered its payload to a target cell. This method can accelerate development of effective and highly specific LNPs for delivery of therapeutics to target cells or target tissues. This technology can be expanded to the use of LNPs to deliver mRNA vaccines. For example, an mRNA encoding a vaccine protein also encodes a unique peptide barcode. In addition to sequencing peptide barcodes to determine whether a particular LNP delivered its payload to a specific cell, peptide barcode sequencing is used to determine whether a particular mRNA vaccine candidate was delivered to a specific cell. These approaches can be coupled to generate data indicative of the efficacy of the LNP delivery and the translation efficacy of the mRNA payload.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method of identifying a protein of interest, the method comprising:
providing a fusion polypeptide comprising a protein of interest fused to a peptide barcode, wherein the peptide barcode is indicative of the protein of interest;
contacting the fusion polypeptide with a cleaving agent, wherein the cleaving agent cleaves the peptide barcode from the fusion polypeptide comprising the protein of interest; and
sequencing the peptide barcode to identify the protein of interest.

2. A method of identifying a protein of interest, the method comprising:
expressing a fusion polypeptide comprising a protein of interest fused to a peptide barcode, wherein the peptide barcode is indicative of the protein of interest;
contacting the fusion polypeptide with a cleaving agent, wherein the cleaving agent cleaves the peptide barcode from the fusion polypeptide comprising the protein of interest; and
sequencing the peptide barcode to identify the protein of interest.

3. The method of claim 2, wherein expressing the fusion polypeptide comprises expressing an expression cassette comprising a gene and a nucleic acid barcode, wherein the gene encodes the protein of interest and the nucleic acid barcode encodes the peptide barcode, wherein expressing the expression cassette produces the fusion polypeptide.

4. The method of any one of claims 1-3, wherein the sequencing comprises detecting a series of signals specific to the peptide barcode.

5. The method of any one of claims 1-4, wherein the sequencing comprises sequencing the peptide barcode at a single molecule level.

6. A method of protein quantification, the method comprising:
expressing an expression cassette comprising a gene and a nucleic acid barcode, wherein the gene encodes a protein of interest and the nucleic acid barcode encodes a peptide barcode, wherein expressing the expression cassette produces the protein of interest conjugated to the peptide barcode;
contacting the protein of interest conjugated to the peptide barcode with a cleaving agent, wherein the cleaving agent removes the peptide barcode from the protein of interest; and
sequencing the peptide barcode by detecting a series of signals specific to the peptide barcode.

7. The method of any one of claims 1-6, further comprising contacting the peptide barcode, prior to sequencing, with a conjugating agent, wherein the conjugating agent conjugates the peptide barcode to a linker.

8. The method of claim 7, wherein the linker immobilizes the peptide barcode in a reservoir.

9. The method of any one of claims 4-8, wherein the series of signals specific to the peptide barcode are generated by contacting the peptide barcode with one or more barcode recognition molecules.

10. The method of claim 9, wherein at least one signal within the series of signals is separated from another signal within the series of signals by an interpulse duration (IPD), pulse duration, and/or fluorescence lifetime that is characteristic of an association rate of barcode recognition molecule binding.

11. The method of claim 9 or 10, wherein at least one of the one or more barcode recognition molecules comprises a detectable label.

12. The method of claim 11, wherein the detectable label is a luminescent label, a fluorescent label, or a conductivity label.

13. The method of claim 11 or 12, wherein the detectable label is a fluorophore or a dye.

14. The method of any one of claims 9-13, further comprising, after contacting the peptide barcode with the one or more barcode recognition molecules:
contacting the peptide barcode with a cleaving protein, wherein the cleaving protein removes one amino acid residue from the peptide barcode, allowing another barcode recognition molecule to contact the peptide barcode.

15. The method of claim 14, wherein the cleaving protein is an aminopeptidase.

16. The method of any one of claims 6-15, wherein expressing is in vivo or in vitro.

17. A method of sample preparation, the method comprising:
expressing a fusion polypeptide comprising a protein of interest fused to a first tag and a peptide barcode, wherein the first tag and the peptide barcode are fused to different termini of the protein of interest;
contacting the fusion polypeptide with a second tag under conditions suitable for conjugating the second tag to the peptide barcode, forming a first complex;
immobilizing the first complex to an immobilizing support that binds the first tag;
contacting the immobilized first complex with a third tag under conditions suitable for conjugating the third tag to the second tag, forming an immobilized second complex; and
contacting the immobilized second complex with a cleaving enzyme that removes the peptide barcode conjugated to the second and third tags from the immobilized second complex comprising the protein of interest.

18. A method of sample preparation, the method comprising:
expressing an expression cassette comprising a first tag, a gene, and a nucleic acid barcode, wherein the gene encodes a protein of interest and the nucleic acid barcode encodes a peptide barcode, wherein expressing the expression cassette produces the protein of interest conjugated to the first tag and the peptide barcode;
contacting the protein of interest conjugated to the first tag and the peptide barcode with a second tag and a tagging enzyme and incubating the protein of interest conjugated to the first tag and the peptide barcode with the second tag and the tagging enzyme for a sufficient time to allow the tagging enzyme to enzymatically conjugate the second tag to the peptide barcode, forming a first complex;
contacting the first complex with an immobilizing support that binds the first tag and incubating the first complex with the immobilizing support for a sufficient time to allow the immobilizing support to bind the first tag;
contacting the immobilized first complex with a third tag and incubating the immobilized first complex with the third tag for a sufficient time to allow the third tag and the first complex to undergo a chemical reaction that binds the third tag to the first complex, forming an immobilized second complex;
contacting the immobilized second complex with a cleaving enzyme that removes the peptide barcode bound to the second and third tags from the immobilized protein of interest; and
sequencing the peptide barcode by detecting a series of signals specific to the peptide barcode.

19. The method of claim 17 or 18, wherein the first tag comprises a protein tag or a ligand tag.

20. The method of any one of claims 17-19, wherein the first tag comprises a protein tag, and wherein the immobilizing support comprises a binding partner of the protein tag.

21. The method of claim 20, wherein the binding partner of the protein tag is a ligand or a protein that binds the protein tag.

22. The method of any one of claims 17-21, wherein the first tag comprises a Halo tag.

23. The method of any one of claims 18-22, wherein the second tag comprises a substrate for the tagging enzyme.

24. The method of any one of claims 18-23, wherein the tagging enzyme is Sortase A.

25. The method of claim 24, wherein the second tag comprises a substrate for Sortase A.

26. The method of claim 24 or 25, wherein the second tag comprises a Sortase A-reactive nucleophile.

27. The method of any one of claims 18-26, wherein the second tag comprises a triglycine peptide.

28. The method of claim 27, wherein the second tag comprises a triglycine-azide.

29. The method of any one of claims 17-28, wherein the immobilizing support is a microbead.

30. The method of claim 29, wherein the microbead is a nickel bead, a nickel-charged affinity resin, an agarose bead, or an iron-based bead.

31. The method of any one of claims 17-30, wherein the third tag comprises a protein tag.

32. The method of claim 31, wherein the third tag comprises a streptavidin protein.

33. The method of any one of claims 17-32, wherein the third tag comprises a click chemistry moiety.

34. The method of claim 33, wherein the click chemistry moiety is a dibenzocyclooctyl (DBCO) moiety, a trans-cyclooctene (TCO) moiety, a tetrazine moiety, an azide moiety, an alkyne moiety, an aldehyde moiety, an isocyanate moiety, an N-hydroxysuccinimide moiety, a thiol moiety, an alkene moiety, a bicyclononyne moiety, or a thiamine pyrophosphate moiety.

35. The method of any one of claims 18-34, wherein the chemical reaction is click chemistry.

36. The method of any one of claims 18-35, wherein the cleaving enzyme is a protease.

37. The method of any one of claims 17-36, wherein the third tag immobilizes the peptide barcode in a reservoir.

38. The method of any one of claims 18-37, wherein the series of signals specific to the peptide barcode are generated by contacting the peptide barcode with one or more barcode recognition molecules.

39. The method of claim 38, wherein at least one signal within the series of signals is separated from another signal within the series of signals by an interpulse duration (IPD), pulse duration, and/or fluorescence lifetime that is characteristic of an association rate of barcode recognition molecule binding.

40. The method of claim 38 or 39, wherein at least one of the one or more barcode recognition molecules comprises a detectable label.

41. The method of claim 40, wherein the detectable label is a luminescent label, a fluorescent label, or a conductivity label.

42. The method of claim 40 or 41, wherein the detectable label is a fluorophore or a dye.

43. The method of any one of claims 38-42, further comprising, after the peptide barcode has been contacted with the one or more barcode recognition molecules:
contacting the peptide barcode with a cleaving protein, wherein the cleaving protein removes one amino acid residue from the peptide barcode, allowing another barcode recognition molecule to contact the peptide barcode.

44. The method of claim 43, wherein the cleaving protein is an aminopeptidase.

45. The method of any one of claims 18-44, wherein expressing is in vivo or in vitro.

46. A method of sequencing error-resistant peptide barcodes, the method comprising:
(i) contacting a peptide barcode with one or more recognition molecules, wherein each recognition molecule recognizes one or more amino acid residues on the peptide barcode, and wherein the peptide barcode has an exposed N-terminus amino acid residue;
(ii) incubating the one or more recognition molecules with the peptide barcode for a sufficient time to allow the recognition molecule that recognizes the N-terminus amino acid to bind the N-terminus amino acid and produce a signal specific to the recognition molecule; and
(iii) contacting the peptide barcode with a cleaving agent that removes the N-terminus amino acid and exposes a subsequent N-terminus amino acid.

47. The method of claim 46, further comprising repeating steps (ii) and (iii) until each amino acid residue on the peptide barcode has been exposed and contacted with its corresponding recognition molecule;
wherein the peptide barcode is incubated with five separate recognition molecules;
wherein: the first recognition molecule recognizes leucine (L), isoleucine (I), and valine (V); the second recognition molecule recognizes arginine (R); the third recognition molecule recognizes phenylalanine (F), tyrosine (Y), and tryptophan (W); the fourth recognition molecule recognizes glutamine (Q) and asparagine (N); and the fifth recognition molecule recognizes alanine (A) and serine (S).

48. The method of claim 46 or 47, further comprising applying a statistical model to correlate the peptide barcode to a series of signals produced by the interaction between one recognition molecule and one exposed N-terminus amino acid residue.

49. The method of any one of claims 46-48, wherein the peptide barcode will produce the same series of signals if a subset of amino acids are switched to another subset of amino acids.

50. The method of claim 49, wherein the peptide barcode will produce the same series of signals if:
the peptide barcode comprises a leucine (L) and the leucine (L) is changed to an isoleucine (I) or a valine (V);
the peptide barcode comprises an isoleucine (I) and the isoleucine is changes to a leucine (L) or a valine (V);
the peptide barcode comprises a valine (V) and the valine (V) is changed to a leucine (L) or an isoleucine (I);
the peptide barcode comprises a phenylalanine (F) and the phenylalanine (F) is changed to a tyrosine (Y) or a tryptophan (W);
the peptide barcode comprises a tyrosine (Y) and the tyrosine (Y) is changed to a phenylalanine (F) or a tryptophan (W);
the peptide barcode comprises a tryptophan (W) and the tryptophan (W) is changed to a phenylalanine (F) or a tyrosine (Y);
the peptide barcode comprises a glutamine (Q) and the glutamine (Q) is changed to an asparagine (N);
the peptide barcode comprises an asparagine (N) and the asparagine (N) is changed to a glutamine (Q);
the peptide barcode comprises an alanine (A) and the alanine (A) is changed to a serine (S); and/or
the peptide barcode comprises a serine (S) and the serine (S) is changed to an alanine (A).

51. A method comprising:
expressing a library of fusion polypeptides, each fusion polypeptide of the library comprising a protein of interest fused to a peptide barcode, wherein the peptide barcode is indicative of the protein of interest to which it is fused;
attaching to the fusion polypeptides of the library of fusion polypeptides a loading complex;
immobilizing the library of fusion polypeptides to a surface;
screening the library of fusion polypeptides for biological activity;
selecting one or more fusion polypeptides of the library of fusion polypeptides based on biological activity;
contacting the library of fusion polypeptides with a labeled binding reagent;
detecting signals indicative of binding between the labeled binding reagent and the protein of interest of at least one fusion polypeptide of the library;
cleaving the peptide barcode from the at least one fusion polypeptide comprising the protein of interest;
sequencing the peptide barcode to identify the protein of interest of the at least one fusion polypeptide.

52. The method of claim 51, wherein the loading complex is a dibenzocyclooctyl (DBCO) moiety.

53. The method of claim 51 or 52, wherein the biological activity is antigen binding affinity, antibody binding affinity, receptor binding affinity, target protein binding affinity, or another biological activity.

54. A method comprising:
expressing a library of fusion polypeptides, each fusion polypeptide of the library comprising a protein of interest fused to a peptide barcode, wherein the peptide barcode is indicative of the protein of interest to which it is fused;
immobilizing the library of fusion polypeptides to a surface;
contacting the library of fusion polypeptides with a labeled binding reagent;
detecting signals indicative of binding between the labeled binding reagent and the protein of interest of at least one fusion polypeptide of the library;
cleaving the peptide barcode from the at least one fusion polypeptide comprising the protein of interest;
sequencing the peptide barcode to identify the protein of interest of the at least one fusion polypeptide; and
determining one or more biophysical characteristics of the protein of interest of the at least one fusion polypeptide based on the detecting and the sequencing.

55. A method of identifying a target analyte in a region of a sample, the method comprising:
(i) contacting a sample with an affinity reagent conjugated to a peptide barcode indicative of a target analyte to which the affinity reagent binds;
(ii) releasing the peptide barcode from the affinity reagent in a first region of the sample; and
(iii) sequencing the peptide barcode to identify the target analyte in the first region of the sample.

56. The method of claim 55, wherein, after step (ii), the peptide barcode remains conjugated to the affinity reagent in a second region of the sample.

57. The method of claim 56, further comprising releasing the peptide barcode from the affinity reagent in the second region of the sample.

58. The method of claim 57, wherein the sequencing comprises identifying the target analyte in each of the first and second regions of the sample.

59. The method of any one of claims 55-58, wherein the target analyte is a protein or a nucleic acid.

60. The method of claim 59, wherein the target analyte is a monomeric or multimeric protein.

61. The method of claim 59, wherein the target analyte is a DNA or RNA molecule, optionally wherein the target analyte is a gene transcript.

62. The method of any one of claims 55-61, wherein the affinity reagent is an antibody or antigen-binding fragment thereof, an antibody-drug conjugate (ADC), an immunohistochemistry (IHC)-compatible antibody, a nanobody, an aptamer, or an antisense oligonucleotide.

63. The method of any one of claims 55-62, wherein the peptide barcode is conjugated to the affinity reagent via a linker.

64. The method of claim 63, wherein the linker comprises a cleavage site.

65. The method of claim 64, wherein the peptide barcode is released from the affinity reagent by contacting the linker with a cleaving agent, optionally wherein the cleaving agent is a cleaving enzyme, further optionally wherein the cleaving enzyme is an endopeptidase.

66. The method of claim 63, wherein the linker is a photocleavable linker.

67. The method of claim 66, wherein the peptide barcode is released from the affinity reagent by exposing the linker to photocleaving light.

68. The method of claim 67, wherein the photocleaving light is ultraviolet radiation or a laser.

69. The method of any one of claims 55-68, further comprising washing the sample between step (i) and step (ii).

70. The method of any one of claims 55-69, further comprising imaging the sample prior to sequencing the peptide barcode.

71. The method of any one of claims 55-70, wherein the sequencing comprises determining a concentration of the target analyte in the first region of the sample.

72. The method of any one of claims 55-71, wherein the sequencing generates sequencing reads associated with the presence of the target analyte in the first region of the sample.

73. The method of claim 72, further comprising mapping the sequencing reads to a spatial representation of the sample.

74. The method of any one of claims 55-73, wherein the sample is a biological sample.

75. The method of claim 74, wherein the biological sample is fixed.

76. The method of claim 74 or 75, wherein the biological sample is a cell sample.

77. The method of any one of claims 74-76, wherein the biological sample is a tissue sample.

78. The method of claim 77, wherein the tissue sample is a formalin-fixed paraffin-embedded (FFPE) tissue sample.

79. The method of any one of claims 55-78, wherein the sample is contacted with a plurality of affinity reagents conjugated to different peptide barcodes indicative of different target analytes.

80. A method of identifying target analytes in a sample, the method comprising:
(i) contacting a sample with a plurality of affinity reagents conjugated to different peptide barcodes indicative of different target analytes;
(ii) releasing one or more peptide barcodes from one or more respective affinity reagents in a first region of the sample;
(iii) releasing one or more peptide barcodes from one or more respective affinity reagents in a second region of the sample; and
(iv) sequencing the one or more peptide barcodes released from each of the first and second regions of the sample.

81. The method of claim 80, wherein the sequencing comprises determining an abundance of at least one target analyte in the first region relative to the second region.

82. The method of claim 80 or 81, wherein the sequencing generates sequencing reads associated with the presence of different target analytes in the first and second regions of the sample.

83. The method of claim 82, further comprising mapping the sequencing reads to a spatial representation of the sample.

84. The method of claim 83, wherein the spatial representation is indicative of localization of different target analytes in different regions of the sample.

85. A method of identifying a therapeutic agent in a region of a sample, the method comprising:
(i) contacting a sample with a therapeutic agent conjugated to a peptide barcode indicative of the therapeutic agent;
(ii) releasing the peptide barcode from the therapeutic agent in a first region of the sample; and
(iii) sequencing the peptide barcode to identify the therapeutic agent in the first region of the sample.

86. The method of claim 85, wherein the therapeutic agent comprises an affinity reagent.

87. The method of claim 86, wherein the affinity reagent is an antibody or antigen-binding fragment thereof, an antibody-drug conjugate (ADC), an immunohistochemistry (IHC)-compatible antibody, a nanobody, an aptamer, or an antisense oligonucleotide.

88. The method of claim 86 or 87, wherein the affinity reagent is an ADC.

89. The method of any one of claims 85-88, wherein, after step (ii), the peptide barcode remains conjugated to the therapeutic agent in a second region of the sample.

90. The method of claim 89, further comprising releasing the peptide barcode from the therapeutic agent in the second region of the sample.

91. The method of claim 90, wherein the sequencing comprises identifying the therapeutic agent in each of the first and second regions of the sample.

92. The method of any one of claims 85-91, wherein the peptide barcode is conjugated to the therapeutic agent via a linker.

93. The method of claim 92, wherein the linker comprises a cleavage site.

94. The method of claim 93, wherein the peptide barcode is released from the therapeutic agent by contacting the linker with a cleaving agent, optionally wherein the cleaving agent is a cleaving enzyme, further optionally wherein the cleaving enzyme is an endopeptidase.

95. The method of claim 92, wherein the linker is a photocleavable linker.

96. The method of claim 95, wherein the peptide barcode is released from the therapeutic agent by exposing the linker to photocleaving light.

97. The method of claim 96, wherein the photocleaving light is ultraviolet radiation or a laser.

98. The method of any one of claims 85-97, further comprising washing the sample between step (i) and step (ii).

99. The method of any one of claims 85-98, further comprising imaging the sample prior to sequencing the peptide barcode.

100. The method of any one of claims 85-99, wherein the sequencing comprises determining a concentration of the therapeutic agent in the first region of the sample.

101. The method of any one of claims 85-100, wherein the sequencing generates sequencing reads associated with the presence of the therapeutic agent in the first region of the sample.

102. The method of claim 101, further comprising mapping the sequencing reads to a spatial representation of the sample.

103. The method of any one of claims 85-102, wherein the sample is a biological sample.

104. The method of claim 103, wherein the biological sample is fixed.

105. The method of claim 103 or 104, wherein the biological sample is a cell sample or a serum sample.

106. The method of any one of claims 103-105, wherein the biological sample is a tissue sample.

107. The method of claim 106, wherein the tissue sample is a formalin-fixed paraffin-embedded (FFPE) tissue sample.

108. A method of evaluating an antibody-drug conjugate (ADC) in a subject, the method comprising:
(i) providing a sample of a subject receiving an ADC, wherein the ADC is conjugated to a peptide barcode;
(ii) releasing the peptide barcode from the ADC in the sample;
(iii) sequencing the peptide barcode; and
(iv) evaluating the ADC based on the sequencing.

109. The method of claim 108, wherein the evaluating comprises determining a concentration of the ADC in the sample based on the sequencing.

110. The method of claim 109, wherein the evaluating comprises comparing the concentration of the ADC in the sample to a control sample.

111. The method of claim 110, wherein the control sample is a sample of the subject prior to receiving the ADC.

112. The method of claim 110, wherein the control sample is a sample of the subject at a different time point after receiving the ADC.

113. The method of any one of claims 108-112, wherein step (ii) comprises:
releasing the peptide barcode from the ADC in a first region of the sample, wherein the peptide barcode remains conjugated to the ADC in a second region of the sample.

114. The method of claim 113, further comprising releasing the peptide barcode from the ADC in the second region of the sample.

115. The method of claim 114, wherein the sequencing comprises sequencing the peptide barcode released from the ADC in each of the first and second regions of the sample.

116. The method of claim 115, wherein the evaluating comprises:
determining a first concentration of the ADC in the first region of the sample based on the sequencing; and
determining a second concentration of the ADC in the second region of the sample based on the sequencing.

117. The method of any one of claims 108-116, wherein the ADC comprises a drug component conjugated to an antibody component via the peptide barcode.

118. The method of any one of claims 108-117, wherein the peptide barcode is conjugated to the ADC via a linker.

119. The method of claim 118, wherein the linker comprises a cleavage site.

120. The method of claim 119, wherein the peptide barcode is released from the ADC by contacting the linker with a cleaving agent, optionally wherein the cleaving agent is a cleaving enzyme, further optionally wherein the cleaving enzyme is an endopeptidase.

121. The method of claim 118, wherein the linker is a photocleavable linker.

122. The method of claim 121, wherein the peptide barcode is released from the ADC by exposing the linker to photocleaving light.

123. The method of claim 122, wherein the photocleaving light is ultraviolet radiation or a laser.

124. The method of any one of claims 108-123, wherein the sample is a serum sample of the subject.

125. The method of any one of claims 108-123, wherein the sample is a tissue sample of the subject.

126. The method of any one of claims 108-125, wherein the subject is a human.

127. The method of any one of claims 108-125, wherein the subject is a non-human animal.

128. A method of identifying delivery to a target cell, the method comprising:
contacting a cell with a delivery agent that comprises a polynucleotide, wherein the polynucleotide encodes a protein of interest fused to a peptide barcode;
contacting the cell with a cleaving agent configured to cleave the peptide barcode from the protein of interest; and
detecting the peptide barcode to identify uptake of the polynucleotide by the cell.

129. The method of claim 128, wherein the delivery agent is a lipid nanoparticle that encapsulates the polynucleotide.

130. A method of evaluating nanoparticle delivery to a target cell, the method comprising:
contacting a cell with a plurality of nanoparticles having different lipid compositions, wherein each nanoparticle encapsulates a polynucleotide, wherein the polynucleotide encodes a protein of interest fused to a peptide barcode indicative of lipid composition of the nanoparticle;
contacting the cell with a cleaving agent configured to cleave the peptide barcode from the protein of interest; and
sequencing the peptide barcode to identify the lipid composition of the nanoparticle.

131. A method of evaluating protein production, the method comprising:
contacting a cell with an mRNA molecule encoding a protein of interest and a peptide barcode;
contacting the cell with a cleaving agent configured to cleave the peptide barcode from the protein of interest; and
detecting the peptide barcode to identify production of the protein of interest.

132. The method of any one of claims 128-131, wherein the contacting is performed under conditions suitable for expression of the polynucleotide within the cell.


SEQUENCE LISTING (68 sequences)

SEQ ID NO: 1 - protein, 36 aa, synthetic construct
  VARIANT 2..5: note = may be absent
  VARIANT 7..36: note = may be absent
GGGGGSGGGGGSGGGGGSGGGGGSGGGGGSGGGGGS

SEQ ID NO: 2 - protein, 7 aa, synthetic construct
  MOD_RES 7: note = Glycine modified by azide (N3)
LPETGGG

SEQ ID NO: 3 - protein, 7 aa, synthetic construct
LPETGGG

SEQ ID NO: 4 - protein, 7 aa, synthetic construct
LPETGXX

SEQ ID NO: 5 - protein, 4 aa, synthetic construct
GGGR

SEQ ID NO: 6 - protein, 5 aa, synthetic construct
TGGGR

SEQ ID NO: 7 - protein, 4 aa, synthetic construct
  MOD_RES 4: note = Lysine modified by azide (N3)
GGGK

SEQ ID NO: 8 - protein, 5 aa, synthetic construct
RLIFA

SEQ ID NO: 9 - protein, 4 aa, synthetic construct
LIFA

SEQ ID NO: 10 - protein, 5 aa, synthetic construct
LNRQF

SEQ ID NO: 11 - protein, 5 aa, synthetic construct
LQRQF

SEQ ID NO: 12 - protein, 5 aa, synthetic construct
LNRQY

SEQ ID NO: 13 - protein, 5 aa, synthetic construct
FNLWL

SEQ ID NO: 14 - protein, 5 aa, synthetic construct
YNLWL

SEQ ID NO: 15 - protein, 5 aa, synthetic construct
LLLLR

SEQ ID NO: 16 - protein, 5 aa, synthetic construct
VVVVR

SEQ ID NO: 17 - protein, 12 aa, synthetic construct
LRFAFLNQADDD

SEQ ID NO: 18 - protein, 12 aa, synthetic construct
LFANFRAHPDDD

SEQ ID NO: 19 - protein, 12 aa, synthetic construct
LNLARFRMMDDD

SEQ ID NO: 20 - protein, 12 aa, synthetic construct
RFNLNAFAADDD

SEQ ID NO: 21 - protein, 12 aa, synthetic construct
RFNANRLAADDD

SEQ ID NO: 22 - protein, 12 aa, synthetic construct
RARANLFAADDD

SEQ ID NO: 23 - protein, 12 aa, synthetic construct
RANFALRMMDDD

SEQ ID NO: 24 - protein, 12 aa, synthetic construct
FLNRLFAHPDDD

SEQ ID NO: 25 - protein, 12 aa, synthetic construct
FLAFRNRMMDDD

SEQ ID NO: 26 - protein, 12 aa, synthetic construct
FRALANAHPDDD

SEQ ID NO: 27 - protein, 12 aa, synthetic construct
FARLFNFAADDD

SEQ ID NO: 28 - protein, 12 aa, synthetic construct
NAFLRLRMMDDD

SEQ ID NO: 29 - protein, 4 aa, synthetic construct
LRMF

SEQ ID NO: 30 - protein, 4 aa, synthetic construct
LFAH

SEQ ID NO: 31 - protein, 4 aa, synthetic construct
LNLA

SEQ ID NO: 32 - protein, 6 aa, synthetic construct
HHHHHH

SEQ ID NO: 33 - protein, 9 aa, synthetic construct
GGSHHHHHH

SEQ ID NO: 34 - protein, 10 aa, synthetic construct
HHHHHHHHHH

SEQ ID NO: 35 - protein, 11 aa, synthetic construct
GHHHHHHHHHH

SEQ ID NO: 36 - protein, 15 aa, synthetic construct
GLNDFFEAQKIEWHE

SEQ ID NO: 37 - protein, 19 aa, synthetic construct
GGGSGLNDFFEAQKIEWHE

SEQ ID NO: 38 - protein, 27 aa, synthetic construct
GGGSGGGSGGGSGLNDFFEAQKIEWHE

SEQ ID NO: 39 - protein, 54 aa, synthetic construct
GGGSGGGSGGGSGLNDFFEAQKIEWHEGGGSGGGSGGGSGLNDFFEAQKIEWHE

SEQ ID NO: 40 - protein, 56 aa, synthetic construct
GSGGGSGGGSGGGSGLNDFFEAQKIEWHEGGGSGGGSGGGSGLNDFFEAQKIEWHE

SEQ ID NO: 41 - protein, 38 aa, synthetic construct
GHHHHHHHHHHGGGSGGGSGGGSGLNDFFEAQKIEWHE

SEQ ID NO: 42 - protein, 65 aa, synthetic construct
GHHHHHHHHHHGGGSGGGSGGGSGLNDFFEAQKIEWHEGGGSGGGSGGGSGLNDFFEAQKIEWHE

SEQ ID NO: 43 - protein, 67 aa, synthetic construct
GGSHHHHHHHHHHGGGSGGGSGGGSGLNDFFEAQKIEWHEGGGSGGGSGGGSGLNDFFEAQKIEWHE

SEQ ID NO: 44 - protein, 66 aa, synthetic construct
GSHHHHHHHHHHGGGSGGGSGGGSGLNDFFEAQKIEWHEGGGSGGGSGGGSGLNDFFEAQKIEWHE

SEQ ID NO: 45 - protein, 61 aa, synthetic construct
GGGSGGGSGGGSGLNDFFEAQKIEWHEGGGSGGGSGGGSGLNDFFEAQKIEWHEGHHHHHH

SEQ ID NO: 46 - protein, 6 aa, synthetic construct
LPETGG

SEQ ID NO: 47 - DNA, 3559 bp, synthetic construct
  source 1..3559: mol_type = other DNA
cgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccgctaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgctagtggtgctagccccgcgaaattaatacgactcactatagggtctagaagaaataattttgtttaactttaagaaggagatatacatatggcggagattggcacaggatttccatttgatccccactatgttgaggttcttggtgaacgtatgcactatgttgacgttggaccacgtgacggaacccctgttttatttttgcatggtaatccgactagtagctacgtttggcgcaacatcatcccacacgtcgcccctactcaccgttgtatcgcaccggacttgatcggtatggggaagtccgacaagccagacttaggctatttctttgacgaccatgtccgtttcatggacgcgtttatcgaggcccttgggttagaagaggttgtacttgtcattcacgactgggggtccgctcttggctttcattgggctaaacgtaaccccgagcgcgtcaaggggatcgcctttatggagtttatccgccccatccctacctgggatgagtggccggaatttgcgcgtgaaacgtttcaagccttccgcacaactgatgttggccgcaaattaattatcgatcagaacgtgttcatcgaggggacattgccaatgggagtggttcgtccgttgaccgaagttgaaatggaccattatcgcgaaccgtttctgaacccggtcgatcgtgaacccttatggcgctttcctaacgaattgccgattgctggtgaacctgcgaacattgtcgcattagtagaggaatacatggactggttacatcaatcccccgttcctaaattgttgttttggggaacgccaggcgtattaatcccgccagcggaggccgcacgccttgcgaagtcacttcctaattgcaaagcagtggatattggcccaggcttgaatcttttgcaagaggataaccctgatctgatcgggtcggaaattgcgcgttggttatcgacccttgagatttcgggcaccagcgaaccaacaactgaggacttgtatttccaatctgacaacgcaatcgctagagacggctacgtctcgggtggcggatctatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggtaggtcttcgagaagacctttgcctgaaaccggcggacatcaccaccatcatcactgactcgagtaaggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcactggccgaccggggtcgagcactgact

SEQ ID NO: 48 - protein, 4 aa, synthetic construct
RMFN

SEQ ID NO: 49 - protein, 4 aa, synthetic construct
RMAH

SEQ ID NO: 50 - protein, 4 aa, synthetic construct
FLNR

SEQ ID NO: 51 - protein, 4 aa, synthetic construct
FLAH

SEQ ID NO: 52 - protein, 4 aa, synthetic construct
FRMA

SEQ ID NO: 53 - protein, 4 aa, synthetic construct
FARL

SEQ ID NO: 54 - protein, 4 aa, synthetic construct
NAHF

SEQ ID NO: 55 - protein, 4 aa, synthetic construct
RLIF

SEQ ID NO: 56 - protein, 4 aa, synthetic construct
RIFL

SEQ ID NO: 57 - protein, 4 aa, synthetic construct
FIRL

SEQ ID NO: 58 - protein, 4 aa, synthetic construct
LFIR

SEQ ID NO: 59 - protein, 4 aa, synthetic construct
ILRF

SEQ ID NO: 60 - protein, 8 aa, synthetic construct
EFLNRFYK

SEQ ID NO: 61 - protein, 12 aa, synthetic construct
ENRLCYYLGATK

SEQ ID NO: 62 - protein, 9 aa, synthetic construct
DQFRLAGGK

SEQ ID NO: 63 - protein, 12 aa, synthetic construct
LRYAFAYPDDDK

SEQ ID NO: 64 - protein, 14 aa, synthetic construct
FAQLQARFAADDDK

SEQ ID NO: 65 - protein, 13 aa, synthetic construct
RLAIQFAYPDDDK

SEQ ID NO: 66 - protein, 12 aa, synthetic construct
ARLAFAYPDDDK

SEQ ID NO: 67 - protein, 10 aa, synthetic construct
FQRIALNFAK

SEQ ID NO: 68 - protein, 9 aa, synthetic construct
VRFLEQQNK